(19) United States
(12) Patent Application Publication
(10) Pub. No.: US 2025/0101023 A1
(43) Pub. Date: Mar. 27, 2025

(54) HETEROARYL DERIVATIVE AND USES THEREOF

(71) Applicants: IUCF-HYU (INDUSTRY-UNIVERSITY COOPERATION FOUNDATION HANYANG UNIVERSITY), Seoul (KR); KOREA INSTITUTE OF SCIENCE AND TECHNOLOGY, Seoul (KR)

(72) Inventors: Gu KONG, Seoul (KR); Ratnakar Reddy KUCHUKULLA, Seoul (KR); Su Min KIM, Incheon (KR); So Jeong MOON, Seoul (KR); Woo Young HUR, Seoul (KR); In Jeong HWANG, Seoul (KR)

(73) Assignees: IUCF-HYU (INDUSTRY-UNIVERSITY COOPERATION FOUNDATION HANYANG UNIVERSITY), Seoul (KR); KOREA INSTITUTE OF SCIENCE AND TECHNOLOGY, Seoul (KR)

(21) Appl. No.: 18/730,491

(22) PCT Filed: Jan. 19, 2023
(86) PCT No.: PCT/KR2023/000930

§ 371 Date: Jul. 19, 2024

(30) Foreign Application Priority Data
Jan. 21, 2022 (KR) 10-2022-0009530
Aug. 1, 2022 (KR) 10-2022-0095624

(51) Int. Cl. C07D 473/34; A61K 31/52; A61K 31/5377; A61P 35/00; C07D 473/16; C07D 473/40

(52) U.S. Cl. CPC C07D 473/34; A61K 31/52; A61K 31/5377; A61P 35/00; C07D 473/16; C07D 473/40

(57) ABSTRACT

The present invention relates to a heteroaryl derivative, a stereoisomer thereof, a hydrate thereof, a solvate thereof, or a pharmaceutically acceptable salt thereof, a method for preparing the same, and a pharmaceutical composition for preventing or treating cancer comprising the same as an active ingredient. The heteroaryl derivative of the present invention exhibits high inhibitory activity against overexpressed HER2, and thus, a pharmaceutical composition containing the same as an active ingredient can be usefully used for preventing or treating HER-2 positive cancer.

### TECHNICAL FIELD

The present invention relates to a heteroaryl derivative, a stereoisomer thereof, a hydrate thereof, a solvate thereof, or a pharmaceutically acceptable salt thereof, a method for preparing the same, and a pharmaceutical composition for preventing or treating cancer including the same as an active ingredient.

### BACKGROUND ART

Cancer is often the result of mutations that may occur in numerous genes that play roles in a wide range of cellular processes. In many cases, cancer cells harbor mutations in genes which control processes such as cell growth, division, differentiation, or interactions with the extracellular environment. As an example, mutations which increase the activity of human epidermal growth factor receptor 2 (HER2), a cell surface receptor which promotes cell growth and division, are associated with many cancers.

In general, tumors are resistant to a particular cancer treatment drug, or are sensitive to a particular drug at the initial stage but develop resistance later. The development of resistance is often the result of mutations that alter cellular activity (for example, mutations that constitutively activate signaling molecules) or result in alterations in gene expression (for example, mutations that result in an increase in expression of cell signaling receptors such as HER2). The resistance of such tumors to drugs is consistent with or results from the development of mutations that transform cancer into a more aggressive (for example, metastatic) form. Metastatic cancer is typically correlated with a worse prognosis compared to non-metastatic cancer.

The MOUNTAINEER clinical trial (ClinicalTrials.gov Identifier #NCT03043313) was reported to confirm the combination effect of tucatinib and trastuzumab for the treatment of patients with HER2-positive metastatic colorectal cancer.

Further, HER2 is an important prognostic and predictive factor in invasive breast cancer, and gene amplification is observed in 20 to 25% of breast cancer, resulting in HER2 overexpression. Breast cancer patients with amplification or overexpression of the HER2 gene have a poor prognosis, but are targeted for targeted therapy using trastuzumab, a monoclonal antibody against HER2 (Herceptin, Genentech, South San Francisco, CA, USA). Such amplification or overexpression of the HER2 gene has also been reported in ovarian cancer, prostate cancer, colorectal cancer, pancreatic cancer, and gastric cancer, in addition to breast cancer, and recently, the results of a multi-institutional phase III clinical study (ToGA trial) were published showing that when trastuzumab was administered in combination with existing anticancer drugs (5-fluorouracil or capecitabine and cisplatin) to patients with HER2-positive advanced gastric cancer, the patient survival time was significantly increased compared to a group administered only the existing anticancer drugs, thereby making the status of HER2 an important predictor of treatment not only in breast cancer but also in gastric cancer.

In particular, cancers that exhibit overexpression of HER2 (referred to as HER2 positive cancers) often have a poor prognosis or are resistant to many standard therapies. Therefore, there is a need for a new drug that is effective for treating HER2-positive or metastatic HER2-positive cancer.

### RELATED ART DOCUMENT

### Patent Document

Korean Patent Application Laid-Open No. 10-2016-0131619

### DISCLOSURE

### Technical Problem

The present invention is directed to providing a compound, a stereoisomer thereof, a hydrate thereof, a solvate thereof, or a pharmaceutically acceptable salt thereof, which can be used for preventing or treating cancer.

The present invention is also directed to providing a method for preparing the compound.

The present invention is also directed to providing a pharmaceutical composition for preventing or treating cancer, containing the compound, a stereoisomer thereof, a hydrate thereof, a solvate thereof, or a pharmaceutically acceptable salt thereof as an active ingredient.

The present invention is also directed to providing a method for preventing or treating cancer, the method including administering the compound, a stereoisomer thereof, a hydrate thereof, a solvate thereof, or a pharmaceutically acceptable salt thereof to a subject in need thereof.

The present invention is also directed to providing a use of the compound of Chemical Formula 1, a stereoisomer thereof, a hydrate thereof, a solvate thereof, or a pharmaceutically acceptable salt thereof for preventing or treating cancer.

### Technical Solution

One aspect of the present invention provides a compound of the following Chemical Formula 1, a stereoisomer thereof, a hydrate thereof, a solvate thereof, or a pharmaceutically acceptable salt thereof.

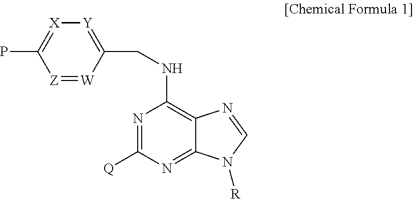

X, Y, Z, and W are each independently CH or N;
P is phenyl or a 5- to 12-membered heteroaryl including one or more heteroatoms of N, S and O, and the phenyl or heteroaryl is unsubstituted or substituted with one or more C₁₋₆ straight or branched alkyls;
Q is phenyl, a 5- to 12-membered heterocycloalkyl including one or more heteroatoms of N, S and O, or a 5- to 12-membered heteroaryl including one or more heteroatoms of N, S and O, the phenyl, the heterocycloalkyl or the heteroaryl may be substituted with one or more non-hydrogen substituents of a C₁₋₆ straight or branched alkyl, a C₁₋₆ alkoxy, a hydroxyl, a halogen, oxo (═O), —NR¹R², and —CONH₂, wherein the C₁₋₆ straight or branched alkyl may be substituted with a hydroxyl, and R¹ and R² are each independently hydrogen or a C₁₋₆ straight or branched alkyl unsubstituted or substituted with a hydroxyl; and
R is a C₁₋₆ straight or branched alkyl, a C₃₋₆ cycloalkyl, or a 3- to 12-membered heterocycloalkyl including one or more heteroatoms of N, S and O.

Another aspect of the present invention provides a method for preparing the compound of Chemical Formula 1.

Still another aspect of the present invention provides a pharmaceutical composition for preventing or treating cancer, containing the compound of the present invention, a stereoisomer thereof, a hydrate thereof, a solvate thereof, or a pharmaceutically acceptable salt thereof as an active ingredient.

Yet another aspect of the present invention provides a method for preventing or treating cancer, the method including administering the compound, a stereoisomer thereof, a hydrate thereof, a solvate thereof, or a pharmaceutically acceptable salt thereof to a subject in need thereof.

Yet another aspect of the present invention provides a use of the compound of Chemical Formula 1, a stereoisomer thereof, a hydrate thereof, a solvate thereof, or a pharmaceutically acceptable salt thereof for preventing or treating cancer.

### Advantageous Effects

The compound provided in an aspect of the present invention, a stereoisomer thereof, a hydrate thereof, a solvate thereof, or a pharmaceutically acceptable salt thereof exhibits high inhibitory activity against HER2, and thus, a pharmaceutical composition including the same as an active ingredient can be usefully used for preventing or treating diseases associated with HER2, such as cancer, particularly HER2-positive cancer.

### DESCRIPTION OF DRAWINGS

FIG. 1 shows the results of western blotting after treating an SK-Br3 cell line with each of the compounds of Examples 1, 2, 4, and 5 for 2 hours, and all of the compounds inhibited Pol II CTD (Ser2) phosphorylation in a dose-dependent manner at a concentration of 0.2 and 1 μM, and induced the degradation of cyclinK, unlike dinaciclib.

FIG. 2 shows the results of performing a western blot analysis after treating SK-Br3 and HCC1954 cell lines with the compounds of Examples 53 and 54 at 40 and 200 nM for (A) 2 hours or (B) 24 hours. All the compounds induced cyclinK degradation, inhibited Pol II CTD (Ser2) phosphorylation, and inhibited the expression of IRS1 and WNT1 in SK-Br3 and HCC1954 cell lines. Control=DMSO, Dina=dinaciclib.

FIG. 3 shows the results of confirming the synergistic effect of combined administration of Example 53 and trastuzumab in (A) SK-Br3 cells and (B) HCC1954 cells. The anti-proliferative activity of trastuzumab was slightly enhanced in both cells by co-treatment with the compound of Example 53 at a concentration of 40 nM. The green line shows the titration of trastuzumab alone, and the blue line shows the titration of trastuzumab in the presence of 40 nM Example 53.

FIG. 4 shows the docking results of the compound of Example 53 using the CDK12-DDB1 complex X-ray crystal structure (pdb id: 6td). CDK12, DDB1 and Example 53 are shown in blue, green and purple, respectively, and the labeled residues are those predicted to interact with the compound of Example 53. Predicted hydrogen bonds are indicated by yellow dotted lines.

FIG. 5 shows the kinome-wide inhibitory activity of Example 53 compound (10 μM concentration) against 371 types of human-derived wild-type kinases. The % residual activity for each kinase was visualized with a web-based visualization tool TREEspot™ (https://www.discoverx.com/services/drug-discovery-development-services/treespot-data-analysis).

FIG. 6 shows the results of evaluating the anticancer efficacy of the compound of Example 53 against a trastuzumab-sensitive SK-Br3 cell line (n=8 per group). The compound and antibody were administered intraperitoneally (i.p.) twice a week.

FIG. 7 shows the results of evaluating the anticancer efficacy of the compound of Example 53 in a mouse xenograft model of a trastuzumab-resistant HCC1954 cell line (n=8 per group). The compound and antibody were administered intraperitoneally (i.p.) twice a week.

### MODES OF THE INVENTION

Hereinafter, the present invention will be described in detail with reference to embodiments.

The exemplary embodiments of the present invention may be modified into various other forms, and the scope of the present invention is not limited to the exemplary aspects to be described below. Further, the exemplary embodiments of the present invention are provided to more fully describe the present invention to a person with ordinary skill in the art.

“Including” a certain element throughout the specification means that it does not exclude other elements, but may further include other elements unless otherwise particularly described.

In the structural formulas of the present specification, the symbol “—” bonding an atom and/or a group may mean a single bond and the symbol “═” may mean a double bond. The symbols may be omitted and may also be displayed if necessary, such as when specifying a bonding atom or bonding position.

In the present specification, “linked” between atoms may include not only the case of direct linkage between atoms, but also the case of indirect linkage between atoms by another atoms and/or groups. In this case, other atoms and/or groups may be oxygen, sulfur, a C₁₋₈ alkylamino, a C₁₋₈ alkylene group, or the like, and are not limited thereto, and the atom and/or the group may be substituted or unsubstituted.

In the present specification, “being substituted or unsubstituted” may mean that one hydrogen atom or a plurality of hydrogen atoms is/are unsubstituted or substituted with other atoms or substituents unless otherwise stated. The substituent may be at least one selected from the group consisting of a halogen (chloro (Cl), iodo (I), bromo (Br), fluoro (F)), a C₁₋₁₀ alkyl, a C₂₋₁₀ alkenyl, a C₂₋₁₀ alkynyl, hydroxyl, a C₁₋₁₀ alkoxy, amino, nitro, thiol, thioether, imine, cyano, phosphonato, phosphine, carboxyl, carbamoyl, carbamic acid, acetal, urea, thiocarbonyl, sulfonyl, sulfonamide, ketone, aldehyde, ester, acetyl, acetoxy, amide, oxygen (═O), a haloalkyl (for example, trifluoromethyl), substituted aminoacyl and aminoalkyl, a carbocyclic cycloalkyl, which may be monocyclic or a fused or non-fused polycyclic (for example, cyclopropyl, cyclobutyl, cyclopentyl, or cyclohexyl), or a heterocycloalkyl, which may be monocyclic or a fused or non-fused polycyclic (for example, pyrrolidinyl, piperidinyl, piperazinyl, morpholinyl, or thiazinyl), carbocyclic or heterocyclic, monocyclic or a fused or non-fused polycyclic aryl (for example, phenyl, naphthyl, pyrrolyl, indolyl, furanyl, thienyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothienyl, or benzofuranyl), amino (primary, secondary, or tertiary), aryl, aryloxy, and aryl-alkyl, and is not limited thereto. In addition, each of the exemplified substituents may be unsubstituted or substituted again with a substituent selected from the group of these substituents.

In the present specification, the “halogen” may be F, Cl, Br, or I.

In the present specification, the “alkyl” may mean a straight or branched non-cyclic; cyclic; or saturated hydrocarbon to which they are bonded, unless otherwise described. Furthermore, the “C₁₋₆ alkyl” may mean an alkyl including 1 to 6 carbon atoms. The non-cyclic alkyl may include, as an example, methyl, ethyl, N-propyl, N-butyl, N-pentyl, N-hexyl, N-heptyl, N-octyl, isopropyl, sec-butyl, isobutyl, tert-butyl, isopentyl, 2-methylpentyl, 3-methylpentyl, 4-methylpentyl, 2,3-dimethylbutyl, and the like, but is not limited thereto. The cyclic alkyl may include, as an example, cyclopropyl, cyclobutyl, cyclopentyl, cyclohexyl, cycloheptyl, cyclooctyl, or the like, but is not limited thereto. The alkyl to which the non-cyclic alkyl and the cyclic alkyl are bonded includes, for example, methylcyclopropyl, cyclopropylmethyl, ethylcyclopropyl, cyclopropylethyl, methylcyclobutyl, cyclobutylmethyl, ethylcyclopentyl, cyclopentylmethyl, or the like, but is not limited thereto.

As used herein, the “cycloalkyl” may refer to, particularly, a cyclic alkyl among alkyls, wherein alkyl is the same as defined above.

As used herein, the “cycloalkene” refers to a hydrocarbon in which multiple carbon atoms are bonded like a ring, hydrogen is bonded to each carbon atom, and which has a double bond in the ring but is not aromatic.

As used herein, the “alkoxy” may refer to —(O-alkyl) as an alkyl ether group, wherein alkyl is the same as defined above. Further, a “C₁₋₆ alkoxy” may refer to an alkoxy containing a C₁₋₆ alkyl, that is, —(O—C₁₋₆ alkyl), and as an example, the C₁₋₆ alkoxy may include methoxy, ethoxy, n-propoxy, isopropoxy, n-butoxy, iso-butoxy, sec-butoxy, tert-butoxy, n-pentoxy, and the like, but is not limited thereto.

As used herein, the “heterocycloalkyl” may refer to a hydrocarbon ring including one or more heteroatoms of N, O, and S as an atom forming a ring, and may be saturated or partially unsaturated. Unless otherwise stated, the heterocycloalkyl may be a monocyclic ring or a polycyclic ring such as a spiro ring, a bridged ring or a fused ring. In addition, “3- to 12-membered heterocycloalkyl” may refer to a heterocycloalkyl including 3 to 12 atoms forming a ring, and as an example, the heterocycloalkyl may include pyrrolidine, piperidine, N-methylpiperidine, imidazolidine, pyrazolidine, butyrolactam, valerolactam, imidazolidinone, hydantoin, dioxolane, phthalimide, piperidine, pyrimidine-2,4(1H,3H)-dione, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrahydrothiophene, quinuclidine, tropane, 2-azaspiro[3.3]heptane, (1R,5S)-3-azabicyclo[3.2.1]octane, (1S,4S)-2-azabicyclo[2.2.2]octane, (1R,4R)-2-oxa-5-azabicyclo[2.2.2]octane, or the like, but is not limited thereto.

As used herein, the “alkylamino” may refer to —(NR′R″), wherein R′ and R″ may each be independently selected from the group consisting of hydrogen and a C₁₋₆ alkyl, and the selected R′ and R″ may each be independently substituted or unsubstituted. In addition, the “C₁₋₆ alkylamino” may refer to an amino containing a C₁₋₆ alkyl, that is, —N—H(C₁₋₆ alkyl) or —N—(C₁₋₆ alkyl)₂, and may include dimethylamino, diethylamino, methylethylamino, methylpropylamino, or ethylpropylamino, but is not limited thereto.

As used herein, the “aryl” may refer to an aromatic ring in which one hydrogen is removed from an aromatic hydrocarbon ring, and may be a monocyclic ring or a polycyclic ring. A “6- to 12-membered aryl” may refer to an aryl including 6 to 12 atoms forming a ring, and may include, as an example, phenyl, naphthalenyl, or anthracenyl, but is not limited thereto.

As used herein, the “heteroaryl” may refer to an aromatic ring containing one or more heteroatoms of N, O, and S as an atom forming a ring, and may be a monocyclic ring or a polycyclic ring. Furthermore, a “5- to 12-membered heteroaryl” may refer to a heteroaryl including 5 to 12 atoms forming a ring, and may include, as an example, thienyl, thiophenyl, furinyl, pyrrolyl, pyrazolyl, imidazolyl, thiazolyl, oxazolyl, isothiazolyl, oxadiazolyl, triazolyl, pyridinyl, bipyridinyl, triazinyl, triazolyl, acridyl, pyridazinyl, pyrazinyl, qunolinyl, quinazoline, quinoxalinyl, phenoxazyl, phthalazinyl, pyrimidinyl, pyrido pyrimidinyl, pyrido pyrazinyl, pyrazino pyrazinyl, isoquinoline, indolyl, carbazolyl, imidazopyridazinyl, imidazopyridinyl, imidazopyrimidinyl, pyrazolopyrimidinyl, imidazopyrazinyl or pyrazolopyridinyl, N-arylcarbazolyl, N-heteroarylcarbazolyl, N-alkylcarbazolyl, benzoxazolyl, benzoimidazolyl, benzothiazolyl, benzocarbazolyl, benzothiophenyl, dibenzothiophenyl, thienothiophenyl, benzofuranyl, phenanthrolinyl, isoxazolyl, oxadiazolyl, thiadiazolyl, benzothiazolyl, tetrazolyl, phenothiazinyl, dibenzosilole, dibenzofuranyl, or the like, but is not limited thereto.

As used herein, the “alkeneyl” may refer to a straight, branched, non-cyclic or cyclic hydrocarbon having one or more double bonds, unless otherwise described.

Further, the “C₂₋₆ alkenyl” may refer to an alkenyl including 2 to 6 carbon atoms, and may include, as an example, ethenyl, 1-propenyl, prop-2-en-1-yl [—(CH₂—CH═CH₂)], 2-butenyl, isopropenyl, 3-butenyl, 4-pentenyl, 5-hexenyl, 1-cyclohexenyl, cyclopentadienyl, 1,3-cyclohexadienyl, 1,4-cyclohexadienyl, 1,3-cycloheptadienyl, 1,3,5-cycloheptatrienyl, or the like, but is not limited thereto.

As used herein, the “hydrate” may refer to a compound of the present invention including a stoichiometric or non-stoichiometric amount of water bonded by a non-covalent intermolecular force, or a salt thereof. The hydrate of the compound represented by Chemical Formula 1 of the present invention may include a stoichiometric or non-stoichiometric amount of water bonded by a non-covalent intermolecular force. The hydrate may contain at least 1 equivalent, preferably 1 to 5 equivalents of water. Such a hydrate may be prepared by crystallizing the compound represented by Chemical Formula 1 of the present invention, a stereoisomer thereof, or pharmaceutically acceptable salt thereof from water or a solvent containing water.

As used herein, the “solvate” may refer to a compound of the present invention including a stoichiometric or non-stoichiometric amount of solvent bonded by a non-covalent intermolecular force, or a salt thereof. Preferred solvents in this regard include volatile, non-toxic, and/or solvents suitable for administration to humans.

As used herein, the “isomer” may refer to a compound of the invention, which has the same chemical or molecular formula but is structurally or sterically different, or a salt thereof. Such isomers include all of a structural isomer such as a tautomer, an R or S isomer having an asymmetric carbon center, a stereoisomer such as a geometric isomer (trans, cis), and an optical isomer (enantiomer). All of these isomers and mixtures thereof are also included within the scope of the present invention.

An aspect of the present invention is to provide a compound of the following Chemical Formula 1, a stereoisomer thereof, a hydrate thereof, a solvate thereof, or a pharmaceutically acceptable salt thereof.

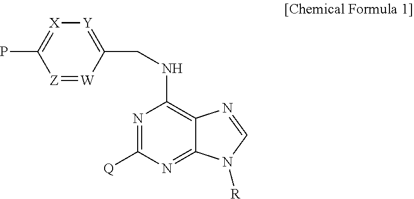

X, Y, Z, and W are each independently CH or N;
P is phenyl or a 5- to 12-membered heteroaryl including one or more heteroatoms of N, S and O, and the phenyl or the heteroaryl is unsubstituted or substituted with one or more C₁₋₆ straight or branched alkyls;
Q is phenyl, a 5- to 12-membered heterocycloalkyl including one or more heteroatoms of N, S and O, or a 5- to 12-membered heteroaryl including one or more heteroatoms of N, S and O, the phenyl, the heterocycloalkyl or heteroaryl may be substituted with one or more non-hydrogen substituents of a C₁₋₆ straight or branched alkyl, a C₁₋₆ alkoxy, a hydroxyl, a halogen, oxo (═O), —NR¹R², and —CONH₂, wherein the C₁₋₆ straight or branched alkyl may be substituted with a hydroxyl, and R¹ and R² are each independently hydrogen or a C₁₋₆ straight or branched alkyl unsubstituted or substituted with a hydroxyl; and
R may be a C₁₋₆ straight or branched alkyl, a C₃₋₆ cycloalkyl, or a 3- to 12-membered heterocycloalkyl including one or more heteroatoms of N, S and O.

In an exemplary embodiment of the present invention, in the compound represented by Chemical Formula 1,
one of X, Y, Z and W is N, and the others are CH,
P is phenyl or a 5- to 12-membered heteroaryl including one or more Ns as a heteroatom, and the phenyl or the heteroaryl is unsubstituted or substituted with one or more C₁₋₃ straight or branched alkyls,
Q is phenyl, a 5- to 12-membered heterocycloalkyl including one or more heteroatoms of N and O, or a 5- to 12-membered heteroaryl including at least one N as a heteroatom, wherein the phenyl, the heterocycloalkyl or the heteroaryl may be substituted with one or more non-hydrogen substituents of a C₁₋₃ straight or branched alkyl, a C₁₋₃ alkoxy, a hydroxyl, a halogen, oxo (═O), —NR¹R², and —CONH₂, wherein the C₁₋₃ straight or branched alkyl may be substituted with a hydroxyl, and R¹ and R² are each independently hydrogen or a C₁₋₃ straight or branched alkyl unsubstituted or substituted with a hydroxyl; and
R may be a C₁₋₃ straight or branched alkyl, a C₃₋₆ cycloalkyl, or a 3- to 6-membered heterocycloalkyl including at least one O as a heteroatom.

In an exemplary embodiment of the present invention, in the compound represented by Chemical Formula 1,
one of X, Y, Z and W is N, and the others are CH,
P is phenyl, pyridine, pyrazole, furan, thiophene, thiazole or indazole, and the phenyl, pyridine, pyrazole, furan, thiophene, thiazole or indazole is unsubstituted or substituted with one or more C₁₋₃ straight or branched alkyls,
Q is phenyl, pyridine, pyrazole, triazole, indole, indazole, benzimidazole, quinoline, thiazole, pyrimidine, morpholine or piperidine, wherein the phenyl, pyridine, pyrazole, triazole, indole, indazole, benzimidazole, quinoline, thiazole, pyrimidine, morpholine or piperidine may be substituted with a C₁₋₃ straight or branched alkyl, a C₁₋₃ alkoxy, a hydroxyl, a halogen, oxo (═O), —NR¹R², and —CONH₂, wherein the C₁₋₃ straight or branched alkyl may be substituted with a hydroxyl, and R¹ and R² are each independently hydrogen or a C₁₋₃ straight or branched alkyl substituted with a hydroxyl; and
R may be a C₁₋₃ straight or branched alkyl, a C₃₋₆ cycloalkyl, or oxane.

In an exemplary embodiment of the present invention, in the compound represented by Chemical Formula 1,
one of X, Y, Z and W is N, and the others are CH,
P is phenyl, pyridine, pyrazole, furan, thiophene, thiazole or indazole, and the phenyl, pyridine, pyrazole, furan, thiophene, thiazole or indazole is unsubstituted or substituted with one or more C₁₋₃ straight or branched alkyls,
Q is phenyl, pyridine, pyrazole, triazole, indole, indazole, benzimidazole, quinoline, thiazole, pyrimidine, morpholine, or piperidine,
when Q is phenyl, the phenyl is unsubstituted or substituted with a hydroxyl, —NH₂, —NHR³, or —CONH₂, wherein R³ is a C₁₋₃ hydroxyalkyl,
when Q is pyridine, pyrazole, triazole, indole, indazole, benzimidazole, quinoline, thiazole, or pyrimidine, the pyridine, pyrazole, triazole, indole, indazole, benzimidazole, quinoline, thiazole, or pyrimidine is unsubstituted or substituted with one or more non-hydrogen substituents of a C₁₋₃ straight or branched alkyl, a C₁₋₃ alkoxy, a halogen, oxo (═O), —NH₂, —NHR⁴, and —CONH₂, wherein the C₁₋₃ straight or branched alkyl may be substituted with a hydroxyl, and R⁴ is a C₁₋₃ hydroxyalkyl, and
when Q is morpholine or piperidine, the morpholine or piperidine is unsubstituted or substituted with a C₁₋₃ hydroxyalkyl, and
R may be a C₁₋₃ straight or branched alkyl, a C₃₋₆ cycloalkyl, or oxane.

In an exemplary embodiment of the present invention, in the compound represented by Chemical Formula 1,

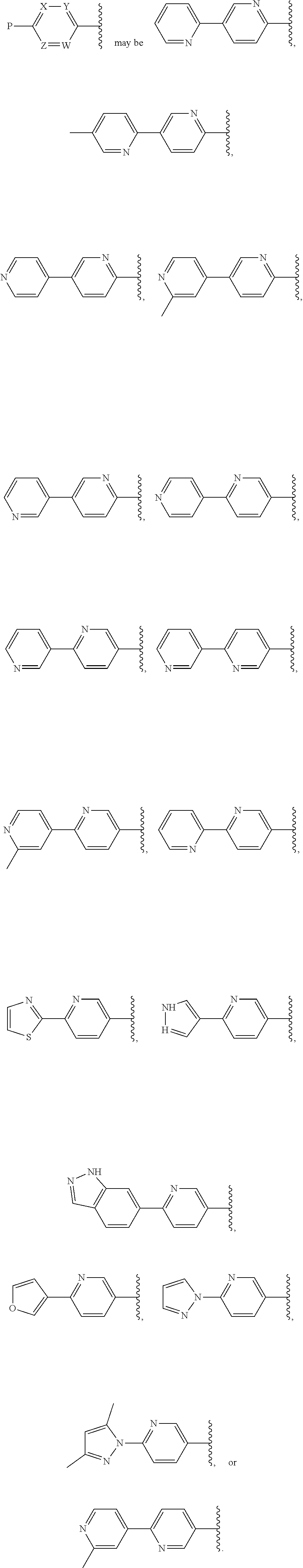

In an exemplary embodiment of the present invention, in the compound represented by Chemical Formula 1,
Q may be phenyl,

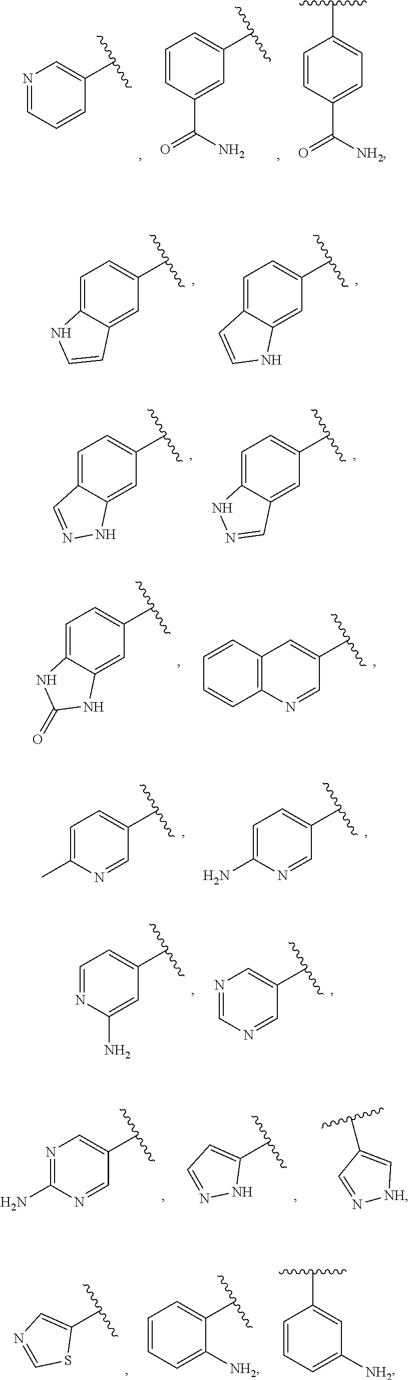

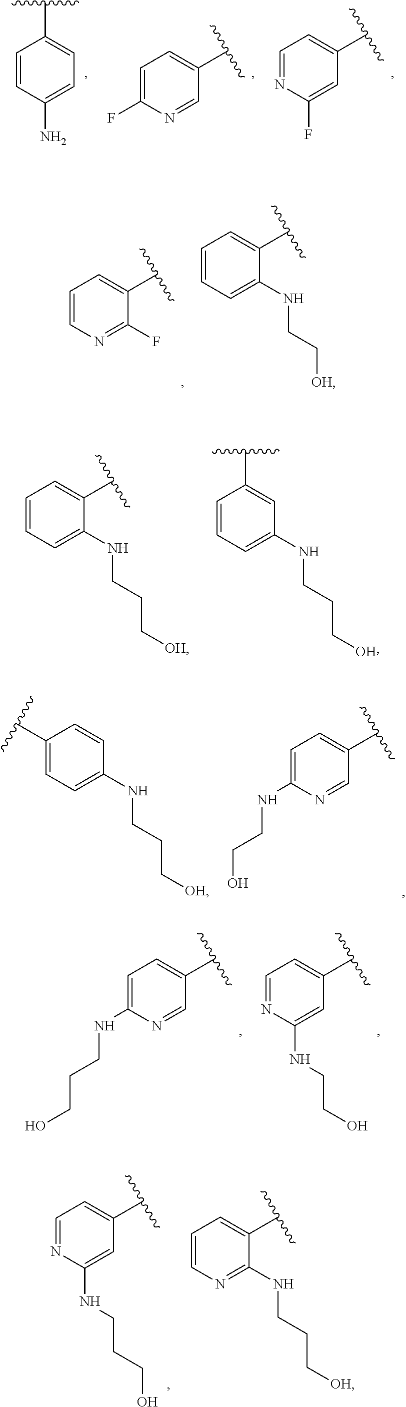

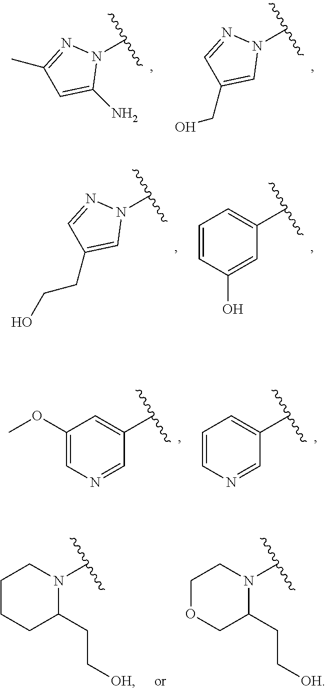

In an exemplary embodiment of the present invention, in the compound represented by Chemical Formula 1,

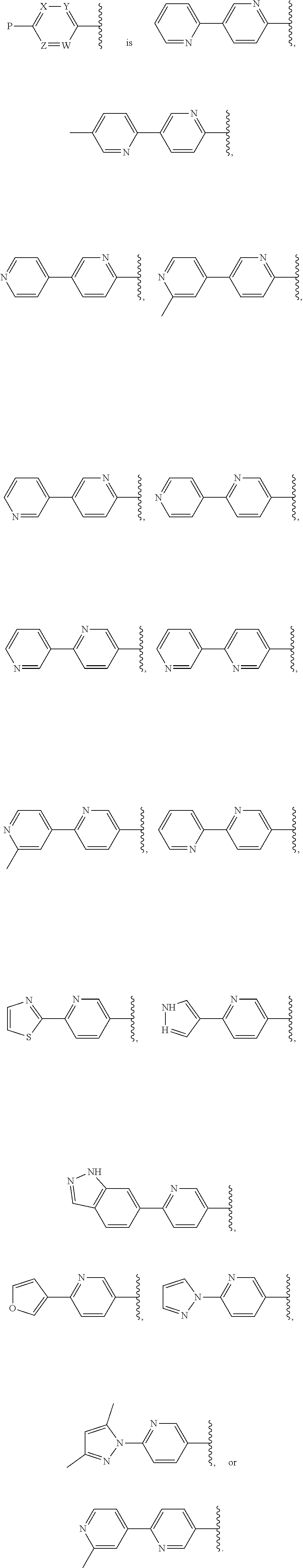

Q is phenyl,

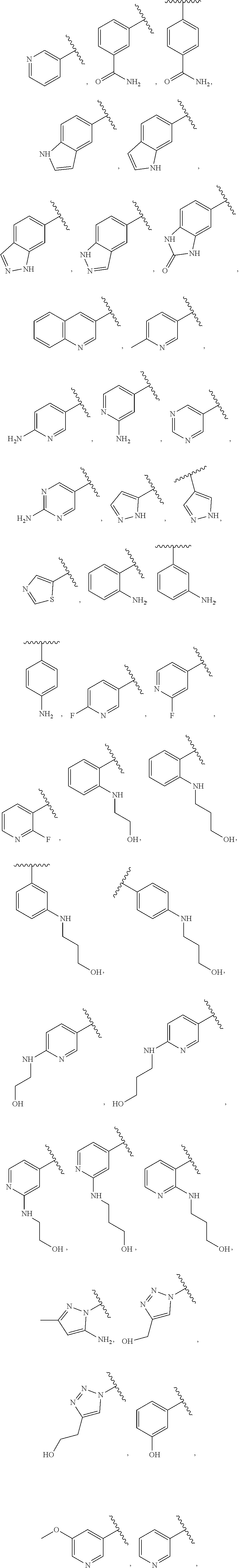

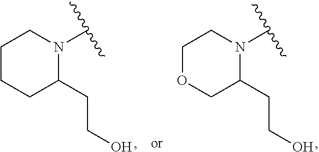 [SMILES: *N1CCCCC1CCO ; *N1CCOCC1CCO]

R may be methyl, ethyl, propyl, isopropyl, a C₃₋₆ cycloalkyl, or oxane.

Examples of the compound of Chemical Formula 1 according to the present invention include the compounds of Examples 1 to 66 enumerated in the following Examples, stereoisomers thereof, hydrates thereof, solvates thereof, or pharmaceutically acceptable salts thereof.

The compound represented by Chemical Formula 1 of the present invention may be used in the form of a pharmaceutically acceptable salt thereof. In particular, the pharmaceutically acceptable salt may be an acid addition salt formed by a free acid.

Here, the acid addition salt may be obtained from an inorganic acid such as hydrochloric acid, nitric acid, phosphoric acid, sulfuric acid, hydrobromic acid, hydroiodic acid, nitrous acid, and phosphorous acid, a non-toxic organic acid such as aliphatic mono and dicarboxylates, phenyl-substituted alkanoates, hydroxy alkanoates and alkanedionates, aromatic acids, and aliphatic and aromatic sulfonic acid, and an organic acid such as trifluoroacetic acid, acetate, benzoic acid, citric acid, lactic acid, maleic acid, gluconic acid, methanesulfonic acid, 4-toluenesulfonic acid, tartaric acid, and fumaric acid. Types of such pharmaceutically acceptable salts may include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butine-1,4-dioate, hexane-1,6-dioate, benzoate, chlorobenzoate, methylbenzoate, dinitro benzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, O-hydroxybutyrate, glycolate, maleate, tartrate, methanesulfonate, propanesulfonate, naphthalene-1-sulfonate, naphthalene-2-sulfonate, mandelate, and the like. The acid addition salt may be prepared by a typical method, for example, by dissolving a derivative of Chemical Formula 1 in an organic solvent such as methanol, ethanol, acetone, methylene chloride, and acetonitrile, adding an organic acid or an inorganic acid thereto, and filtering and drying the resulting precipitate, or may be prepared by distilling a solvent and an excess amount of acid under reduced pressure, and then drying the solvent and the acid to crystallize the resulting product under an organic solvent. Further, the pharmaceutically acceptable salt may be a salt or metal salt obtained using a base. As an example of the metal salt, an alkali metal or alkaline earth metal salt may be obtained by dissolving the compound in an excess alkali metal hydroxide or alkaline earth metal hydroxide solution, filtering the non-soluble compound salt, evaporating the filtrate, and drying the resulting product. As the alkali metal salt, a sodium, potassium or calcium salt may be pharmaceutically suitable. In addition, a salt corresponding thereto may be obtained by reacting an alkali metal or alkaline earth metal salt with a suitable silver salt (for example, silver nitrate).

Furthermore, the present invention may be not only the compound represented by Chemical Formula 1 and a pharmaceutically acceptable salt thereof, but also a stereoisomer thereof, particularly an enantiomer, and may be a hydrate and/or solvate that may be prepared therefrom.

Another aspect of the present invention provides a method for preparing a compound of Chemical Formula 1.

Specifically, the method may include:
preparing a compound of Chemical Formula 3 from a compound of Chemical Formula 2;
preparing a compound of Chemical Formula 4 from the compound of Chemical Formula 3; and
preparing a compound of Chemical Formula 1 from the compound of Chemical Formula 4:

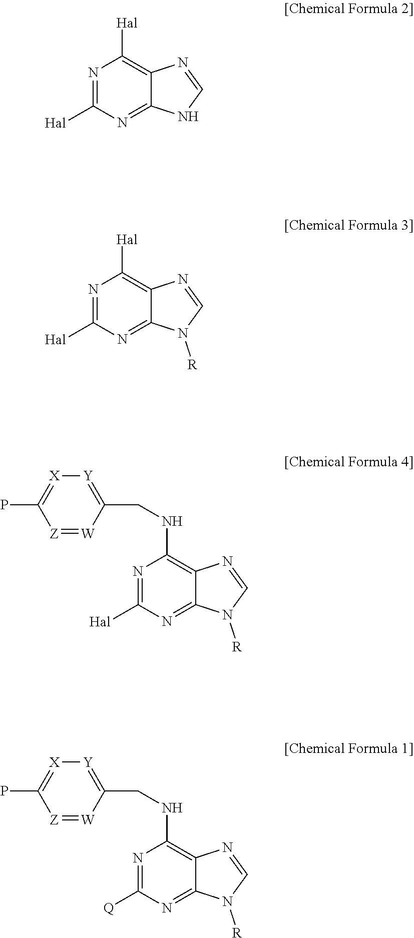

in the above formulae, Hal is a halogen which is a leaving group, and X, Y, Z, W, P, Q, and R are each the same as defined above.

The preparation method according to the present invention will be described in detail in the following compound synthesis methods and examples.

Still another aspect of the present invention may provide a pharmaceutical composition for preventing or treating cancer, including the compound of Chemical Formula 1, a stereoisomer thereof, a hydrate thereof, a solvate thereof, or a pharmaceutically acceptable salt thereof as an active ingredient.

It has been confirmed through experiments that the compound represented by Chemical Formula 1 of the present invention may exhibit CDK12 inhibitory activity, and specifically, exhibits inhibitory activity against CDK12/cyclinK.

Further, the compound represented by Chemical Formula 1 of the present invention may exhibit inhibitory activity against HER2.

Accordingly, yet another aspect of the present invention may provide a pharmaceutical composition for preventing or treating an HER2-related disease, including the compound of Chemical Formula 1, a stereoisomer thereof, a hydrate thereof, a solvate thereof, or a pharmaceutically acceptable salt thereof as an active ingredient.

The type of cancer may include, but is not limited to, any of a number of cancers known to be associated with HER2, including solid tumors, specifically HER2-positive cancer. For example, the cancer may be selected from the group consisting of colorectal cancer, gastric cancer, lung cancer (for example, non-small cell lung cancer (NSCLC)), biliary tract cancer (for example, cholangiocarcinoma, gallbladder cancer), bladder cancer, esophageal cancer, melanoma, ovarian cancer, liver cancer, prostate cancer, pancreatic cancer, colon cancer, head and neck cancer, uterine cancer, breast cancer, and cervical cancer. Specifically, the cancer to be prevented or treated by the pharmaceutical composition of the present invention may be selected from the group consisting of colorectal cancer, esophageal cancer, gastric cancer, cholangiocarcinoma, non-small cell lung cancer, bladder cancer, breast cancer and biliary tract cancer, and more specifically, the cancer may be breast cancer, particularly HER2-positive breast cancer.

In addition, the present invention may also be used to treat patients with HER2-positive breast cancer and HER2-positive breast cancer or triple-negative breast cancer types that are resistant to HER2-antibody therapeutic agents (for example, Herceptin).

The pharmaceutical composition for preventing or treating cancer of the present invention may be used for clinical administration, and may be prepared so as to be administered in various dosage forms for oral and parenteral administration.

A pharmaceutical composition for preventing or treating cancer, including the compound of Chemical Formula 1, a stereoisomer thereof, a hydrate thereof, a solvate thereof, or a pharmaceutically acceptable salt thereof as an active ingredient, may be administered as an individual therapeutic agent or used in combination with other therapeutic agents currently being used.

Yet another aspect of the present invention provides a method for preparing or treating cancer, the method including administering the compound of Chemical Formula 1, a stereoisomer thereof, a hydrate thereof, a solvate thereof, or a pharmaceutically acceptable salt thereof to a subject in need thereof.

Yet another aspect of the present invention is to provide a use of the compound of Chemical Formula 1 or a stereoisomer thereof, a solvate thereof, or a pharmaceutically acceptable salt thereof for preventing or treating cancer.

Meanwhile, the novel compound represented by Chemical Formula 1 according to the present invention can be formulated in various forms according to the purpose. The following illustrates several formulation methods in which the compound represented by Chemical Formula 1 according to the present invention is contained as an active ingredient, but the present invention is not limited thereto.

Hereinafter, the present invention will be described in detail with reference to examples and experimental examples.

However, the following examples and comparative examples are only for illustrating the present invention, and the content of the present invention is not limited by the following examples.

It is to be understood that the symbols (for example, X, Y, Z, V, W, R, and the like) shown in the chemical formulae in the following reaction schemes are merely exemplarily shown in order to describe each step of the synthesis process of a specific compound and are not related to the symbols described in the claims or other parts of the specification.

### [Compound Synthesis Method]

Synthesis Method of Intermediates (n,n′-bipyridin-5-ylmethanaminiums)

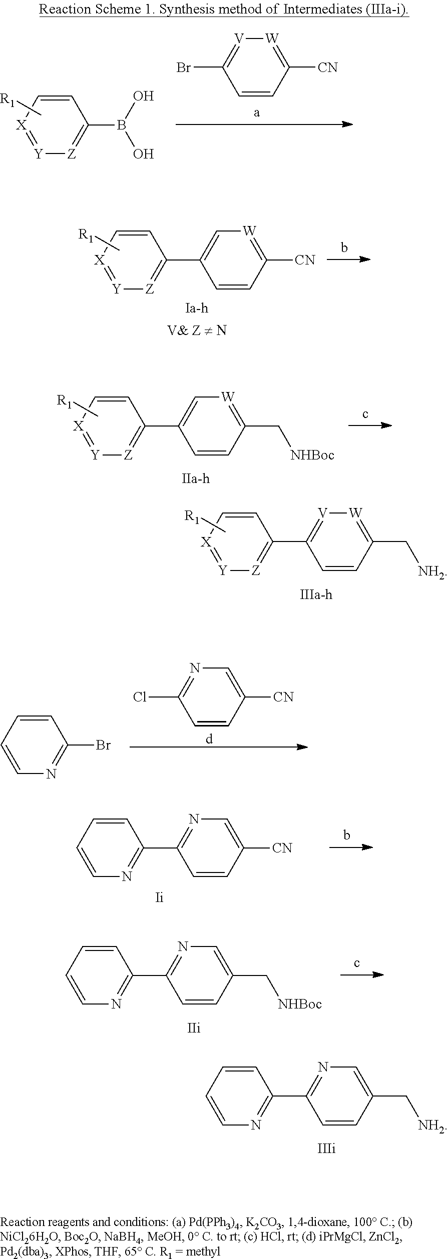

### General Synthesis Method of Ia-h

Under nitrogen gas, aryl bromide (1 eq), pyridylboronic acid (1.2 eq), Pd(PPh₃)₄ (0.05 eq), and a 2 M K₂CO₃ aqueous solution were added to a 1,4-dioxane (45 mL) solvent and heated to 100° C. for 12 hours with vigorous stirring. After cooling, ethyl acetate was added to the mixture, and the resulting mixture was washed with water and brine. The organic layer was dried over MgSO₄, filtered, and concentrated under reduced pressure. The residue was purified by column chromatography using n-hexane:EtOAc to obtain a white solid.

### [2,3′-bipyridine]-6′-carbonitrile (Ia)

¹H NMR (400 MHz, CDCl₃) δ 9.31 (d, J=1.7 Hz, 1H), 8.77 (d, J=4.5 Hz, 1H), 8.50 (dd, J=8.1, 2.2 Hz, 1H), 7.91-7.78 (m, 3H), 7.38 (ddd, J=7.1, 4.8, 1.3 Hz, 1H); ¹³C NMR (101 MHz, DMSO-d₆) δ 151.86, 150.12, 149.19, 137.70, 137.13, 135.19, 132.39, 129.07, 124.33, 121.72, 117.53, 40.15, 39.94, 39.73, 39.52, 39.52, 39.31, 39.10, 38.89; HRMS (ESI+) m/z calcd for C₁₁H₇N₃ [M+H]⁺ 182.0718, found 182.0721.

### [3,3′-bipyridine]-6-carbonitrile (Tb)

¹H NMR (400 MHz, DMSO-d₆) δ 9.17 (d, J=1.9 Hz, 1H), 9.05 (d, J=2.0 Hz, 1H), 8.69 (dd, J=4.8, 1.4 Hz, 1H), 8.45 (dd, J=8.1, 2.3 Hz, 1H), 8.30-8.24 (m, 1H), 8.18 (d, J=8.2 Hz, 1H), 7.59-7.54 (m, 1H); ¹³C NMR (101 MHz, DMSO-d₆) δ 150.18, 149.42, 148.20, 136.27, 135.77, 134.95, 131.80, 131.08, 129.13, 124.07, 117.51, 40.15, 39.94, 39.73, 39.52, 39.52, 39.31, 39.10, 38.90; HRMS (ESI+) m/z calcd for C₁₁H₇N₃ [M+H]⁺ 182.0718, found 182.0721.

### 6′-methyl-[3,3′-bipyridine]-6-carbonitrile (Ic)

¹H NMR (400 MHz, CDCl₃) δ 8.86 (dd, J=2.2, 0.7 Hz, 1H), 8.68 (d, J=2.1 Hz, 1H), 7.94 (dd, J=8.1, 2.3 Hz, 1H), 7.74 (ddd, J=8.0, 5.1, 1.6 Hz, 2H), 7.26 (d, J=8.1 Hz, 1H), 2.58 (s, 3H). ¹³C NMR (101 MHz, CDCl₃+CD₃OD) δ 159.55, 149.14, 147.01, 136.77, 135.27, 135.03, 132.56, 132.23, 131.99, 131.89, 128.96, 128.70, 128.66, 128.54, 124.06, 116.96, 24.48; HRMS (ESI+) m/z calcd for C₁₂H₉N₃ [M+H]⁺: 196.0869, found: 196.0881.

### [3,4′-bipyridine]-6-carbonitrile (Id)

¹H NMR (400 MHz, CDCl₃) δ 8.99 (d, J=1.6 Hz, 1H), 8.79 (d, J=5.1 Hz, 2H), 8.07 (dd, J=8.0, 2.1 Hz, 1H), 7.84 (d, J=8.0 Hz, 1H), 7.53 (d, J=5.3 Hz, 2H); ¹³C NMR (101 MHz, DMSO-d₆) δ 150.54, 149.51, 142.45, 136.04, 132.70, 129.22, 121.71, 117.39, 40.15, 39.94, 39.73, 39.52, 39.52, 39.31, 39.10, 38.89; HRMS (ESI+): m/z calcd for C₁₁H₇N₃ [M+H]⁺ 182.0718, found 182.0721.

### 2′-methyl-[3,4′-bipyridine]-6-carbonitrile (Ie)

¹H NMR (400 MHz, CDCl₃) δ 8.89 (d, J=1.6 Hz, 1H), 8.65-8.52 (m, 1H), 8.05-7.92 (m, 1H), 7.76 (dd, J=8.1, 0.7 Hz, 1H), 7.36-7.24 (m, 2H), 2.60 (s, 3H). ¹³C NMR (101 MHz, CDCl₃) δ 159.87, 150.23, 149.47, 143.57, 137.38, 135.26, 133.79, 132.13, 132.03, 128.57, 128.45, 118.77, 116.97, 24.63.

### [2,3′-bipyridine]-5-carbonitrile (If)

¹H NMR (400 MHz, DMSO-d₆) δ 9.34 (d, J=1.7 Hz, 1H), 9.14 (d, J=1.5 Hz, 1H), 8.71 (dd, J=4.7, 1.4 Hz, 1H), 8.52 (dd, J=8.0, 1.5 Hz, 1H), 8.46 (dd, J=8.3, 2.1 Hz, 1H), 8.30 (d, J=8.3 Hz, 1H), 7.58 (dd, J=8.0, 4.8 Hz, 1H); ¹³C NMR (101 MHz, DMSO-d₆) δ 157.04, 152.72, 151.15, 148.35, 141.21, 134.67, 132.50, 123.98, 120.68, 117.08, 108.12, 40.15, 40.15, 39.94, 39.94, 39.73, 39.73, 39.52, 39.52, 39.52, 39.31, 39.31, 39.10, 39.10, 38.89, 38.89.

### [2,4′-bipyridine]-6-carbonitrile (Ig)

¹H NMR (400 MHz, CDCl₃) δ 9.01 (d, J=1.9 Hz, 1H), 8.81 (s, 2H), 8.11 (dd, J=8.0, 1.7 Hz, 1H), 7.95 (d, J=7.8 Hz, 3H); ¹³C NMR (101 MHz, CDCl₃) δ 157.87, 152.81, 150.79, 144.54, 140.51, 121.37, 120.64, 116.51, 109.92, 77.48, 77.36, 77.16, 77.16, 76.84; HRMS (ESI+) m/z calcd for C₁₁H₇N₃ [M+H]⁺ 182.0718, found 182.0725.

### 2′-methyl-[2,4′-bipyridine]-5-carbonitrile (Ih)

¹H NMR (400 MHz, CDCl₃) δ 8.90 (d, J=1.6 Hz, 1H), 8.58 (dd, J=11.2, 3.3 Hz, 1H), 8.01-7.93 (m, 1H), 7.75 (dd, J=8.1, 0.7 Hz, 1H), 7.34-7.22 (m, 2H), 2.59 (d, J=8.7 Hz, 3H). ¹³C NMR (101 MHz, CDCl₃) δ 159.67, 158.14, 152.64, 150.02, 144.76, 140.32, 120.87, 120.60, 118.36, 116.45, 109.66, 24.57; HRMS (ESI+) m/z calcd for C₁₂H₉N₃ [M+H]⁺: 196.0869, found: 196.0881.

### General Synthesis Method of IIIa-h

Each intermediate Ia-h (about 20 g) was placed in methanol (120 mL) and cooled to 0° C. Di-tert-butyl dicarbonate (2 eq) was added, the suspension was stirred for 15 minutes, and then NiCl₂6H₂O (0.3 eq) was added thereto, and the resulting mixture was stirred for 5 minutes. Thereafter, NaBH₄ (3.5 eq) was added little by little for 30 minutes. After the addition was completed (about 30 minutes), the ice bath was removed and the mixture was stirred overnight while warming to room temperature. After the reaction was completed, diethylenetriamine (1 eq) was added to the stirred mixture. After 15 minutes, methanol was evaporated, 100 mL of a saturated aqueous NaHCO₃ solution was added thereto, the resulting mixture was extracted with ethyl acetate (3×80 mL), and then the organic layer was dried over MgSO₄, evaporated under reduced pressure, and subjected to column chromatography (98% dichloromethane, 2% methanol) to obtain Boc-protected intermediates (IIa-h). Thereafter, each intermediate was dissolved in 50 mL of dichloromethane, the resulting solution was cooled to 4° C., and then 10 mL of 4 N HCl dissolved in 1,4-dioxane was slowly added, and the resulting mixture was stirred at room temperature for 1 hour. The solid eluted from the dichloromethane solvent was filtered and dried to obtain a light brown salt as a pure product (IIIa-h).

### [2,3′-bipyridin]-6′-ylmethanamium (IIIa

¹H NMR (400 MHz, DMSO-d₆) δ 9.33 (d, J=2.0 Hz, 1H), 8.81 (d, J=5.0 Hz, 1H), 8.72 (s, 2H), 8.61 (dd, J=8.2, 2.3 Hz, 1H), 8.23 (ddd, J=13.7, 9.4, 4.8 Hz, 2H), 7.77 (d, J=8.2 Hz, 1H), 7.67 (dd, J=8.9, 3.6 Hz, 1H), 4.29 (q, J=5.7 Hz, 2H); ¹³C NMR (101 MHz, DMSO-d₆) δ 154.79, 150.14, 146.87, 145.77, 142.79, 137.21, 129.93, 125.36, 124.00, 123.31, 42.14, 40.15, 39.94, 39.73, 39.52, 39.52, 39.31, 39.10, 38.89.

### [3,3′-bipyridine]-6-ylmethanamium (IIIb

¹H NMR (400 MHz, DMSO-d₆) δ 9.40 (d, J=1.9 Hz, 1H), 9.14 (d, J=2.1 Hz, 1H), 8.95 (t, J=6.5 Hz, 2H), 8.73 (s, 3H), 8.43 (dd, J=8.2, 2.4 Hz, 1H), 8.15 (dd, J=8.1, 5.6 Hz, 1H), 7.76 (d, J=8.2 Hz, 1H), 4.27 (q, J=5.8 Hz, 2H); ¹³C NMR (101 MHz, DMSO-d₆) δ 154.42, 147.29, 142.82, 141.69, 140.70, 136.14, 135.27, 129.47, 127.10, 122.94, 42.45, 40.15, 39.94, 39.73, 39.52, 39.52, 39.31, 39.10, 38.89.

### (6′-methyl-[3,3′-bipyridin]-6-yl)methanamine (IIIc

¹H NMR (400 MHz, DMSO) δ 9.28 (s, 1H), 9.18 (s, 1H), 8.99-8.84 (m, 4H), 8.55-8.47 (m, 1H), 8.09 (d, J=8.4 Hz, 1H), 7.85 (d, J=8.2 Hz, 1H), 4.29 (q, J=5.3 Hz, 2H), 2.86 (s, 3H). ¹³C NMR (101 MHz, DMSO-d₆) δ 154.40, 153.55, 146.99, 143.90, 139.15, 137.05, 133.14, 129.66, 128.62, 123.77, 42.62, 19.15.

### [3,4′-bipyridine]-6-ylmethanamium (IIId

¹H NMR (400 MHz, DMSO-d₆) δ 9.19 (s, 1H), 8.90 (d, J=5.7 Hz, 2H), 8.45 (d, J=6.0 Hz, 4H), 8.22 (d, J=5.5 Hz, 2H), 7.71 (d, J=8.0 Hz, 1H), 4.32 (q, J=5.9 Hz, 2H); ¹³C NMR (101 MHz, DMSO-d₆) δ 156.30, 152.35, 148.04, 142.38, 136.56, 129.75, 124.26, 123.05, 42.59, 40.15, 39.94, 39.73, 39.52, 39.52, 39.31, 39.10, 38.89.

### (2′-methyl-[3,4′-bipyridin]-6-yl)methanamine (IIIe

¹H NMR (400 MHz, DMSO-d₆) δ 9.26 (d, J=2.1 Hz, 1H), 8.90 (d, J=6.3 Hz, 1H), 8.70 (bs, 3H), 8.54 (dd, J=8.2, 2.1 Hz, 1H), 8.50 (s, 1H), 8.37 (d, J=5.0 Hz, 1H), 7.80 (d, J=8.2 Hz, 1H), 4.32 (q, J=11.3, 5.5 Hz, 2H), 2.84 (s, 3H).

### [2,3′-bipyridine]-5-ylmethanamium (IIIf

¹H NMR (400 MHz, DMSO-d₆) δ 9.51 (d, J=2.0 Hz, 1H), 9.09 (d, J=8.3 Hz, 1H), 8.92 (dd, J=5.5, 1.3 Hz, 1H), 8.89 (d, J=1.7 Hz, 1H), 8.68 (s, 3H), 8.31 (d, J=8.2 Hz, 1H), 8.20 (dd, J=8.2, 2.3 Hz, 1H), 8.07 (dd, J=8.1, 5.5 Hz, 1H), 4.17 (q, J=5.8 Hz, 2H); ¹³C NMR (101 MHz, DMSO-d₆) δ 150.66, 149.87, 142.90, 141.76, 139.82, 139.02, 136.71, 131.30, 127.54, 121.34, 40.15, 39.94, 39.73, 39.52, 39.52, 39.31, 39.10, 38.89.

### [2,4′-bipyridine]-6-ylmethanamium (IIIg

¹H NMR (400 MHz, DMSO-d₆) δ 9.05 (d, J=6.8 Hz, 2H), 8.98 (d, J=1.8 Hz, 1H), 8.93 (s, 3H), 8.74 (d, J=6.8 Hz, 2H), 8.49 (d, J=8.2 Hz, 1H), 8.31 (dd, J=8.2, 2.1 Hz, 1H), 4.19 (q, J=5.8 Hz, 2H); ¹³C NMR (101 MHz, DMSO-d₆) δ 150.66, 149.87, 142.90, 141.76, 139.82, 139.02, 136.71, 131.30, 127.54, 121.34, 40.15, 39.94, 39.73, 39.52, 39.52, 39.31, 39.10, 38.89.

### (2′-methyl-[2,4′-bipyridin]-5-yl)methanamine (IIIh

¹H NMR (400 MHz, DMSO-d₆) δ 8.98 (d, J=1.8 Hz, 1H), 8.90 (d, J=6.3 Hz, 1H), 8.81 (bs, 3H), 8.67 (s, 1H), 8.55 (dd, J=6.3, 1.5 Hz, 1H), 8.47 (d, J=8.2 Hz, 1H), 8.29 (dd, J=8.2, 2.2 Hz, 1H), 4.21 (q, J=5.6 Hz, 2H), 2.85 (d, J=6.9 Hz, 3H).

Synthesis Method of Intermediates (2,6-dichloro-9-alkyl-9H-purine)

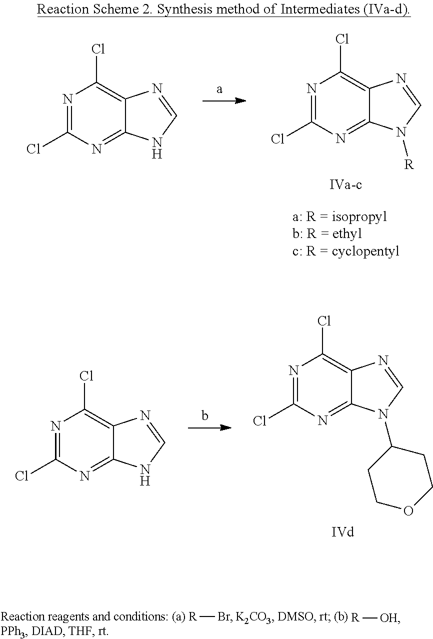

### General Synthesis Method of IVa-c

2,6-Dichloro-9H-purine (10.0 g, 53.2 mmol) and K₂CO₃ (21.9 g, 159 mmol) were dissolved in 70 mL of anhydrous DMSO. Alkyl bromide (133 mmol) was added dropwise to the reaction mixture at room temperature, and the resulting mixture was stirred overnight. When the reaction was completed, the reaction mixture was poured into ice water, extracted with ethyl acetate, and dried over MgSO₄. The concentrated mixture was subjected to column chromatography using n-hexane/ethyl acetate (3:1) to obtain a pure product in a yield of 40 to 70%.

Synthesis Method of 2,6-dichloro-9-(tetrahydro-2H-pyran-4-yl)-9H-purine (IVd)

A mixture of 2,6-dichloro-9H-purine (0.56 g, 3 mmol), 4-hydroxytetrahydropyran (0.455 g, 4.5 mmol) and Ph3P (1.18 g, 4.5 mmol) in THF (20 mL) was stirred at room temperature under nitrogen gas. After 1 hour, DIAD (0.909 g, 4.5 mmol) was added dropwise in an ice bath, and the resulting mixture was stirred at room temperature for 2 days. The solvent was evaporated and the residue was purified by column chromatography (n-hexane:EtOAc:CH₂Cl₂=1:1:0.4) to obtain a product (0.505 g, a yield of 72%).

The synthesis methods of general derivatives, except for some exceptional derivatives, are as follows.

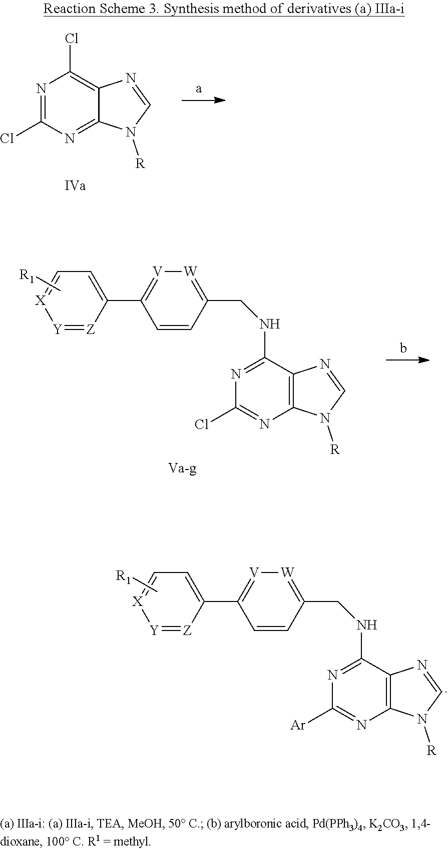

### General Synthesis Method of Intermediates (V)

Each 2,6-dichloro-9-alkylpurine (IVa-d) (37.7 mmol) was dissolved in methanol (20 mL) under nitrogen gas, each n,n′-bipyridin-5-ylmethanaminium (IIIa-i) (1.2 eq) and triethylamine (3 eq) were added thereto at room temperature, and the reaction mixture was heated at 50° C. for 12 hours. After the reaction was completed, methanol was evaporated, the residue was diluted with EtOAc, and then washed with a saturated aqueous NaHCO₃ solution, water, and brine in this order, and then the organic layer was dried over MgSO₄, filtered, and concentrated. The residue was purified by column chromatography (2 to 3% methanol in dichloromethane) to obtain a solid product.

### N-([2,3′-bipyridin]-6′-ylmethyl)-2-chloro-9-isopropyl-9H-purin-6-amine (Va)

¹H NMR (400 MHz, CDCl₃) δ 9.09 (d, J=1.7 Hz, 1H), 8.67 (d, J=4.2 Hz, 1H), 8.26 (dd, J=8.1, 2.2 Hz, 1H), 7.85 (s, 1H), 7.81 (s, 1H), 7.74 (td, J=7.7, 1.7 Hz, 1H), 7.69 (d, J=7.9 Hz, 1H), 7.46 (d, J=8.2 Hz, 1H), 7.26-7.18 (m, 1H), 4.97 (s, 2H), 4.78 (dt, J=13.5, 6.8 Hz, 1H), 1.54 (d, J=6.8 Hz, 6H); ¹³C NMR (101 MHz, CDCl₃) δ157.02, 155.15, 154.55, 154.22, 150.11, 147.50, 138.03, 137.04, 135.19, 133.82, 122.88, 122.14, 120.53, 119.18, 77.48, 77.36, 77.16, 77.16, 76.84, 47.01, 45.45, 22.86; HRMS (ESI+) m/z calcd for C₁₉H₁₈ClN₇ [M+H]⁺ 380.1390, found 380.1391.

### N-([3,3′-bipyridin]-6-ylmethyl)-2-chloro-9-isopropyl-9H-purin-6-amine (Vb)

¹H NMR (400 MHz, CDCl₃) δ 8.79 (d, J=1.8 Hz, 1H), 8.75 (d, J=1.8 Hz, 1H), 8.62 (dd, J=4.8, 1.3 Hz, 1H), 7.87 (s, 1H), 7.83 (dt, J=8.1, 2.5 Hz, 2H), 7.74 (s, 1H), 7.49 (d, J=8.1 Hz, 1H), 7.38 (dd, J=7.7, 4.9 Hz, 1H), 4.97 (s, 2H), 4.84-4.75 (dt, J=13.5, 6.8 Hz, 1H), 1.55 (d, J=6.8 Hz, 6H); ¹³C NMR (101 MHz, CDCl₃) δ 156.59, 155.13, 154.22, 149.35, 148.15, 147.44, 138.05, 135.28, 134.44, 133.28, 132.38, 123.88, 122.42, 119.20, 77.48, 77.36, 77.16, 77.16, 76.84, 47.05, 45.38, 22.87, 22.70; HRMS (ESI+) m/z calcd for C₁₉H₁₈ClN₇ [M+H]⁺ 380.1390, found 380.1392.

### 2-chloro-9-isopropyl-N-((6′-methyl-[3,3′-bipyridin]-6-yl)methyl)-9H-purin-6-amine (Vc)

¹H NMR (400 MHz, CDCl₃) δ 8.80 (d, J=1.7 Hz, 1H), 8.74 (d, J=2.1 Hz, 1H), 7.91-7.84 (m, 2H), 7.81 (dd, J=8.0, 2.4 Hz, 1H), 7.50 (d, J=8.1 Hz, 1H), 7.38 (s, 1H), 7.30 (d, J=7.9 Hz, 1H), 5.01 (s, 1H), 4.85 (dq, J=13.3, 6.7 Hz, 1H), 2.66 (s, 3H), 1.61 (d, J=6.8 Hz, 6H). ¹³C NMR (101 MHz, CDCl₃) δ 158.22, 147.29, 147.21, 137.84, 135.05, 134.84, 132.40, 130.38, 123.53, 46.98, 24.11, 22.83; HRMS (ESI+) m/z calcd for C₂₀H₂₀ClN₇ [M+H]⁺: 394.1547, found: 394.1551.

### N-([3,4′-bipyridin]-6-ylmethyl)-2-chloro-9-isopropyl-9H-purin-6-amine (Vd)

¹H NMR (400 MHz, DMSO-d₆) δ 8.92 (s, 1H), 8.74 (s, 1H), 8.70-8.65 (m, 2H), 8.31 (s, 1H), 8.08 (d, J=8.2 Hz, 1H), 8.02 (d, J=6.1 Hz, 2H), 7.91 (d, J=8.1 Hz, 1H), 4.71 (d, J=5.5 Hz, 2H), 4.67 (d, J=6.7 Hz, 2H), 1.49 (d, J=6.7 Hz, 6H); ¹³C NMR (101 MHz, CDCl₃) δ 155.59, 155.14, 148.08, 139.38, 137.44, 124.03, 77.48, 77.36, 77.16, 76.84, 47.69, 22.85; HRMS (ESI+) m/z calcd for C₁₉H₁₈ClN₇ [M+H]⁺ 380.1390, found 380.1394.

### 2-chloro-9-isopropyl-N-((2′-methyl-[3,4′-bipyridin]-6-yl)methyl)-9H-purin-6-amine (Ve)

¹H NMR (400 MHz, CDCl₃) δ 8.75 (d, J=1.7 Hz, 1H), 8.60 (d, J=4.8 Hz, 1H), 7.85 (dd, J=8.1, 2.2 Hz, 1H), 7.76 (dd, J=10.6, 8.4 Hz, 3H), 7.66 (d, J=5.1 Hz, 1H), 6.84 (s, 1H), 4.92 (s, 1H), 4.81 (dq, J=13.5, 6.8 Hz, 1H), 2.66 (s, 3H), 1.58 (d, J=6.8 Hz, 6H). ¹³C NMR (101 MHz, CDCl₃) δ 159.10, 154.14, 149.64, 149.57, 146.43, 137.98, 136.69, 120.74, 118.20, 47.12, 24.49, 22.75; HRMS (ESI+) m/z calcd for C₂₀H₂₀ClN₇ [M+H]⁺: 394.1547, found: 394.1547.

### N-([2,3′-bipyridin]-5-ylmethyl)-2-chloro-9-isopropyl-9H-purin-6-amine (Vf)

¹H NMR (400 MHz, CDCl₃) δ 9.15 (s, 1H), 8.70 (d, J=1.6 Hz, 1H), 8.63 (s, 1H), 8.27 (d, J=8.0 Hz, 1H), 7.79 (dd, J=8.1, 2.2 Hz, 1H), 7.70 (s, 1H), 7.67 (d, J=8.1 Hz, 1H), 7.37 (dd, J=7.7, 4.8 Hz, 1H), 6.93 (s, 1H), 4.85 (d, J=18.7 Hz, 2H), 4.78 (dt, J=13.6, 6.8 Hz, 1H), 1.53 (d, J=6.8 Hz, 6H); ¹³C NMR (101 MHz, CDCl₃) δ155.13, 154.14, 150.00, 149.76, 148.22, 138.03, 136.79, 134.38, 133.27, 123.72, 120.40, 119.02, 77.48, 77.36, 77.16, 77.16, 76.84, 47.18, 41.87, 22.84; HRMS (ESI+) m/z calcd for C₁₉H₁₈ClN₇ [M+H]⁺ 380.1390, found 380.1394.

### N-([2,4′-bipyridin]-5-ylmethyl)-2-chloro-9-isopropyl-9H-purin-6-amine (Vg)

¹H NMR (400 MHz, CDCl₃) δ 8.80 (s, 1H), 8.23 (s, 1H), 7.87 (dd, J=31.8, 7.4 Hz, 3H), 6.84 (s, 1H), 4.93 (s, 2H), 4.84 (dt, J=13.4, 6.6 Hz, 1H), 1.58 (d, J=6.7 Hz, 6H); ¹³C NMR (101 MHz, CDCl₃) δ 155.03, 152.41, 150.22, 137.15, 135.46, 121.31, 121.31, 77.48, 77.48, 77.36, 77.16, 77.16, 77.16, 76.84, 47.43, 22.88; HRMS (ESI+) m/z calcd for C₁₉H₁₈ClN₇ [M+H]⁺ 380.1390, found 380.1395.

### General Synthesis Method of Example Compounds

A 2,6,9-trisubstituted purine intermediate (for example: Va-g), arylboronic acid (1.2 eq), and Pd(PPh₃)₄ (0.05 eq) were mixed in 1,4-dioxane (2 mL) under nitrogen gas, and 0.5 mL of a 2 M aqueous K₂CO₃ solution was added, and then the mixture was warmed to 100° C. with stirring for 12 hours. After the reaction, the mixture was cooled to room temperature, diluted with ethyl acetate, and washed with water and brine, and then the organic layer was dried over MgSO₄ and concentrated. The residue was purified by silica column chromatography using 4 to 5% methanol in dichloromethane to obtain final compounds.

The synthesis methods, structures and activities (CDK12/cyclinK enzyme activity, inhibitory activity against the trastuzumab-sensitive SK-Br3 cell line and trastuzumab-resistant HCC1954 cell line) of compounds of Examples 1 to 7 are as follows.

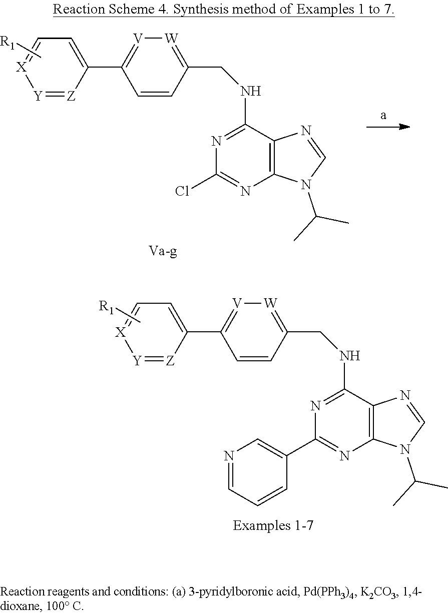

### Example 1: N-([2,3′-bipyridin]-6′-ylmethyl)-9-isopropyl-2-(pyridin-3-yl)-9H-purin-6-amine

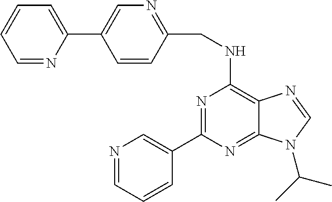 [SMILES: CC(C)n1cnc2c(NCc3ccc(-c4ccccn4)cn3)nc(-c3cccnc3)nc21]

¹H NMR (400 MHz, CDCl₃) δ 9.66 (s, 1H), 9.13 (d, J=1.9 Hz, 1H), 8.69 (d, J=8.0 Hz, 1H), 8.66 (d, J=4.3 Hz, 1H), 8.61 (d, J=3.7 Hz, 1H), 8.23 (dd, J=8.2, 2.2 Hz, 1H), 7.87 (s, 1H), 7.77-7.69 (m, 1H), 7.67 (d, J=7.9 Hz, 1H), 7.51 (d, J=8.2 Hz, 1H), 7.45 (s, 1H), 7.34 (dd, J=7.8, 4.8 Hz, 1H), 7.22 (ddd, J=7.1, 4.8, 1.2 Hz, 1H), 5.14 (s, 2H), 4.88 (dt, J=13.5, 6.8 Hz, 1H), 1.63 (d, J=6.8 Hz, 6H); ¹³C NMR (101 MHz, CDCl₃) δ 158.43, 156.51, 154.67, 154.44, 150.06, 149.91, 149.76, 147.60, 138.43, 137.00, 135.71, 135.10, 134.57, 133.58, 123.21, 122.79, 121.76, 120.51, 119.74, 77.48, 77.36, 77.16, 77.16, 76.84, 47.27, 45.78, 22.77; HRMS (ESI+) m/z calcd for C₂₄H₂₂N₈ [M+H]⁺ 423.2046, found 423.2046.

### Example 2: N-([3,3′-bipyridin]-6-ylmethyl)-9-isopropyl-2-(pyridin-3-yl)-9H-purin-6-amine

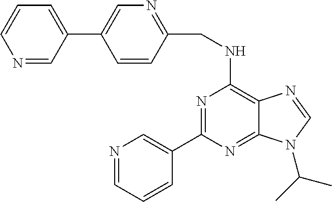 [SMILES: CC(C)n1cnc2c(NCc3ccc(-c4cccnc4)cn3)nc(-c3cccnc3)nc21]

¹H NMR (400 MHz, CDCl₃) δ 9.68 (s, 1H), 8.83 (dd, J=5.1, 2.0 Hz, 2H), 8.73 (d, J=8.0 Hz, 1H), 8.68-8.60 (m, 2H), 7.92-7.82 (m, 3H), 7.55 (d, J=8.1 Hz, 1H), 7.43-7.35 (m, 2H), 6.93 (s, 1H), 5.16 (s, 2H), 4.99-4.88 (m, 1H), 1.67 (d, J=6.8 Hz, 6H); ¹³C NMR (101 MHz, CDCl₃) δ 158.14, 156.55, 154.37, 150.15, 149.92, 149.16, 148.04, 147.38, 138.40, 135.35, 135.07, 134.27, 133.25, 131.98, 123.75, 123.07, 121.97, 119.61, 77.48, 77.16, 76.84, 47.19, 45.64, 22.69; HRMS (ESI+) m/z calcd for C₂₄H₂₂N₈ [M+H]⁺ 423.2046, found 423.2046.

### Example 3: 9-isopropyl-N-((6′-methyl-[3,3′-bipyridin]-6-yl)methyl)-2-(pyridin-3-yl)-9H-purin-6-amine

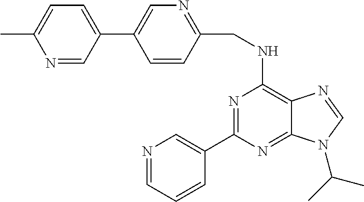 [SMILES: Cc1ccc(-c2ccc(CNc3nc(-c4cccnc4)nc4c3ncn4C(C)C)nc2)cn1]

¹H NMR (400 MHz, CDCl₃) δ 9.61 (s, 1H), 8.72 (d, J=1.9 Hz, 1H), 8.68-8.61 (m, 2H), 8.56 (d, J=3.6 Hz, 1H), 7.80 (s, 1H), 7.75 (dd, J=8.1, 2.2 Hz, 1H), 7.69 (dd, J=8.0, 2.3 Hz, 1H), 7.46 (d, J=8.1 Hz, 1H), 7.29 (dd, J=7.9, 4.8 Hz, 1H), 7.17 (s, 1H), 6.94 (s, 1H), 5.08 (s, 2H), 4.91-4.80 (m, 1H), 2.54 (s, 3H), 1.60 (d, J=6.8 Hz, 6H). ¹³C NMR (101 MHz, CDCl₃) δ 158.20, 157.28, 156.64, 150.23, 150.01, 147.41, 147.38, 138.25, 135.40, 134.97, 134.67, 132.24, 130.40, 123.40, 123.07, 121.90, 47.23, 24.18, 22.74. HRMS (ESI+) m/z calcd for C₂₅H₂₄N₈ [M+H]⁺: 437.2202, found: 437.2206.

### Example 4: N-([3,4′-bipyridin]-6-ylmethyl)-9-isopropyl-2-(pyridin-3-yl)-9H-purin-6-amine

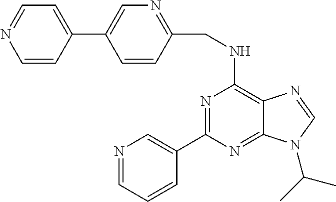 [SMILES: CC(C)n1cnc2c(NCc3ccc(-c4ccncc4)cn3)nc(-c3cccnc3)nc21]

¹H NMR (400 MHz, CDCl₃) δ 9.67 (s, 1H), 8.83 (s, 1H), 8.77 (d, J=7.8 Hz, 1H), 8.68 (d, J=20.1 Hz, 3H), 7.89 (d, J=13.8 Hz, 4H), 7.78 (d, J=8.0 Hz, 1H), 7.44 (s, 1H), 6.40 (s, 1H), 5.07 (s, 2H), 4.94 (dd, J=13.3, 6.4 Hz, 1H), 1.67 (d, J=6.7 Hz, 6H); ¹³C NMR (101 MHz, CDCl₃) δ 158.73, 154.24, 153.84, 149.90, 149.66, 148.25, 138.82, 138.09, 136.56, 134.67, 134.45, 134.31, 129.81, 128.33, 128.22, 123.64, 120.39, 119.21, 77.48, 77.36, 77.16, 76.84, 47.17, 22.82; HRMS (ESI+) m/z calcd for C₂₄H₂₂N₈ [M+H]⁺ 423.2046, found 423.2043.

### Example 5: 9-isopropyl-N-((2′-methyl-[3,4′-bipyridin]-6-yl)methyl)-2-(pyridin-3-yl)-9H-purin-6-amine

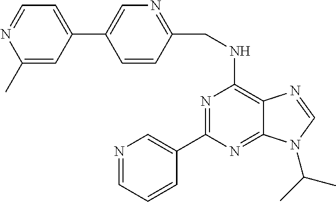 [SMILES: Cc1cc(-c2ccc(CNc3nc(-c4cccnc4)nc4c3ncn4C(C)C)nc2)ccn1]

¹H NMR (400 MHz, CDCl₃) δ 9.61 (d, J=1.4 Hz, TH), 8.79 (d, J=1.8 Hz, TH), 8.64 (dt, J=7.9, 1.9 Hz, TH), 8.57 (dd, J=4.8, 1.7 Hz, TH), 8.51 (d, J=5.2 Hz, TH), 7.84-7.79 (m, 2H), 7.47 (t, J=6.5 Hz, TH), 7.33-7.27 (m, 2H), 7.24 (d, J=5.2 Hz, TH), 6.78 (s, TH), 5.09 (s, 2H), 4.86 (dd, J=13.6, 6.8 Hz, TH), 2.57 (s, 3H), 1.60 (t, J=7.1 Hz, 6H). ¹³C NMR (101 MHz, CDCl₃) δ 159.26, 158.49, 156.59, 154.31, 150.17, 149.94, 149.82, 147.51, 145.39, 138.32, 135.43, 135.06, 134.36, 132.69, 123.10, 121.90, 121.05, 119.69, 118.69, 47.25, 24.56, 22.73. HRMS (ESI+) m/z calcd for C₂₅H₂₄N₈ [M+H]⁺: 437.2202, found: 437.2193.

### Example 6: N-([2,3′-bipyridin]-5-ylmethyl)-9-isopropyl-2-(pyridin-3-yl)-9H-purin-6-amine

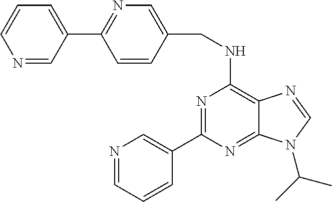 [SMILES: CC(C)n1cnc2c(NCc3ccc(-c4cccnc4)nc3)nc(-c3cccnc3)nc21]

¹H NMR (400 MHz, CDCl₃) δ 9.66 (s, 1H), 9.15 (s, 1H), 8.76 (d, J=1.3 Hz, 1H), 8.73-8.48 (m, 3H), 8.25 (d, J=7.9 Hz, 1H), 7.84 (dd, J=8.1, 1.9 Hz, 1H), 7.80 (s, 1H), 7.66 (d, J=8.1 Hz, 1H), 7.40 (d, J=36.2 Hz, 2H), 6.76 (s, 1H), 5.02 (s, 2H), 4.89 (dt, J=13.5, 6.8 Hz, 1H), 1.62 (t, J=10.5 Hz, 6H); ¹³C NMR (101 MHz, CDCl₃) δ156.72, 154.35, 153.96, 150.34, 149.96, 149.63, 148.26, 138.46, 136.54, 135.50, 134.62, 134.33, 134.16, 123.67, 123.24, 120.42, 119.60, 77.48, 77.16, 76.84, 47.41, 41.87, 22.80; HRMS (ESI+) m/z calcd for C₂₄H₂₂N₈ [M+H]⁺ 423.2046, found 423.2042.

### Example 7: N-([2,4′-bipyridin]-5-ylmethyl)-9-isopropyl-2-(pyridin-3-yl)-9H-purin-6-amine

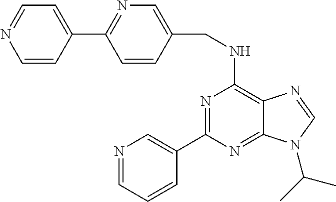 [SMILES: CC(C)n1cnc2c(NCc3ccc(-c4ccncc4)nc3)nc(-c3cccnc3)nc21]

¹H NMR (400 MHz, CDCl₃) δ 9.66 (s, 1H), 8.82 (s, 1H), 8.77-8.67 (m, 3H), 8.65 (s, 1H), 7.87 (d, J=12.6 Hz, 4H), 7.76 (d, J=8.0 Hz, 1H), 7.41 (s, 1H), 6.55 (s, 1H), 5.05 (s, 2H), 4.96-4.88 (m, 1H), 1.66 (d, J=6.6 Hz, 6H); ¹³C NMR (101 MHz, CDCl₃) δ 156.45, 154.30, 153.69, 150.19, 149.74, 149.38, 146.51, 138.64, 136.63, 136.09, 135.29, 123.51, 121.20, 120.87, 77.48, 77.36, 77.16, 76.84, 47.55, 22.85; HRMS (ESI+) m/z calcd for C₂₄H₂₂N₈ [M+H]⁺ 423.2046, found 423.2041.

The process for preparing the compound of Example 8 to the compound of Example 42 is as follows.

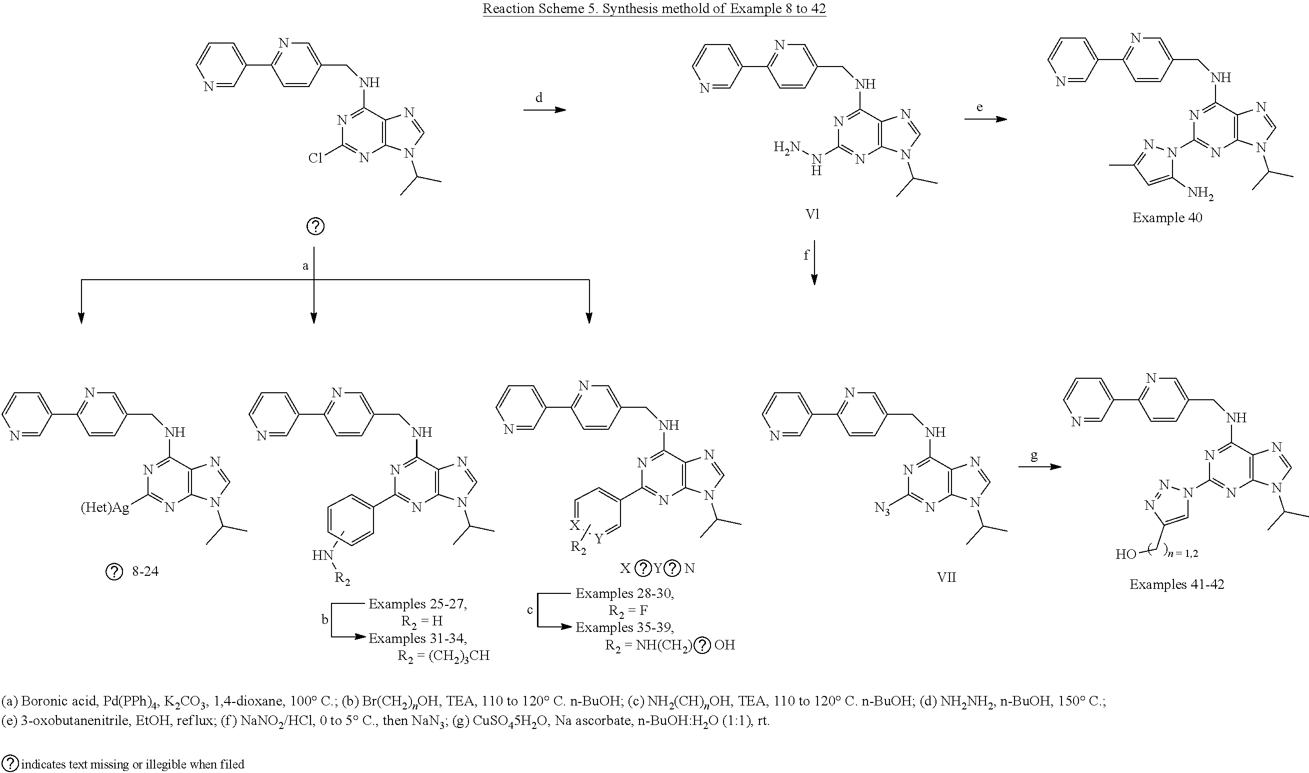

### Synthesis Method of Examples 31 to 34

An anilino compound (Example 25 to 27) (1.0 eq) and trimethylamine (2.0 eq) were added to n-butanol (1.0 mL) in which bromoalkyl alcohol (1.5 eq) was dissolved at room temperature, and the mixture was heated with stirring at 110° C. for 12 hours. After the reaction was completed, the mixture was cooled to room temperature and the solvent was evaporated. The residue was diluted with water and extracted with ethyl acetate (3×50 mL). The combined organic extract was dried over MgSO₄, filtered, and concentrated under reduced pressure. The product was obtained by column chromatography using dichloromethane:methanol=95%: 5%.

### Synthesis Method of Examples 35 to 39

Aminoalkyl alcohol (1.2 eq) and triethylamine (3 eq) were added to a solution of each of Examples 28 to 30 (0.05 mmol) in n-butanol (1 mL) under nitrogen gas at room temperature, and the mixture was reacted in a microwave at 120° C. for 2 hours. After the reaction was completed, the mixture was diluted with ethyl acetate and washed with a saturated aqueous NaHCO₃ solution, water, and brine in this order, and then the organic layer was dried over MgSO₄, filtered, and concentrated under reduced pressure. The residue was purified by column chromatography using 5% methanol in dichloromethane to obtain a compound as a white solid.

### Example 8: N-([2,3′-bipyridin]-5-ylmethyl)-9-isopropyl-2-phenyl-9H-purin-6-amine

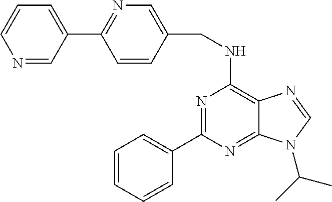 [SMILES: CC(C)n1cnc2c(NCc3ccc(-c4cccnc4)nc3)nc(-c3ccccc3)nc21]

¹H NMR (400 MHz, CDCl₃) δ 9.20 (d, J=1.7 Hz, 1H), 8.86 (d, J=1.8 Hz, 1H), 8.67 (dd, J=4.8, 1.5 Hz, 1H), 8.52 (dd, J=7.8, 1.8 Hz, 2H), 8.39-8.28 (m, 1H), 8.01-7.92 (m, 1H), 7.89 (s, 1H), 7.75 (d, J=8.1 Hz, 1H), 7.55-7.36 (m, 4H), 5.13 (s, 2H), 4.98 (dd, J=13.6, 6.8 Hz, 1H), 1.70 (d, J=6.8 Hz, 6H). ¹³C NMR (101 MHz, CDCl₃) δ158.66, 154.13, 153.85, 149.86, 149.63, 148.21, 138.76, 138.05, 136.52, 134.32, 134.24, 129.72, 128.25, 128.13, 123.57, 120.36, 47.09, 22.76; HRMS (ESI+) m/z calcd for C₂₅H₂₃N₇ [M+H]⁺: 422.2093, found: 422.2097.

### Example 9: 3-(6-(([2,3′-bipyridin]-5-ylmethyl)amino)-9-isopropyl-9H-purin-2-yl)benzamide

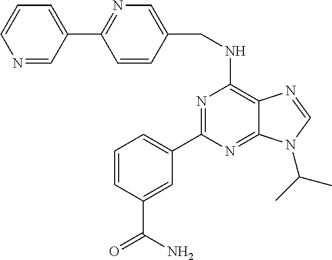 [SMILES: CC(C)n1cnc2c(NCc3ccc(-c4cccnc4)nc3)nc(-c3cccc(C(N)=O)c3)nc21]

¹H NMR (400 MHz, DMSO-d₆) 59.21 (s, 1H), 8.90 (s, 1H), 8.83 (s, 1H), 8.57 (dd, J=26.9, 6.3 Hz, 3H), 8.38 (d, J=8.1 Hz, 1H), 8.33 (s, 1H), 8.10 (s, 1H), 8.01 (s, 2H), 7.93 (d, J=7.7 Hz, 1H), 7.56 (t, J=7.8 Hz, 1H), 7.49 (dd, J=8.0, 4.7 Hz, 1H), 7.45 (s, 1H), 4.88 (dd, J=13.5, 6.8 Hz, 3H), 1.60 (d, J=6.8 Hz, 6H). ¹³C NMR (101 MHz, DMSO-d₆) 5168.55, 165.52, 152.81, 150.16, 148.13, 140.08, 135.07, 134.40, 134.26, 124.21, 121.13, 120.82, 116.88, 46.93, 22.80; HRMS (ESI+) m/z calcd for C₂₆H₂₄N₈O [M+H]⁺: 465.2151, found: 465.2157.

### Example 10: 4-(6-(([2,3′-bipyridin]-5-ylmethyl)amino)-9-isopropyl-9H-purin-2-yl)benzamide

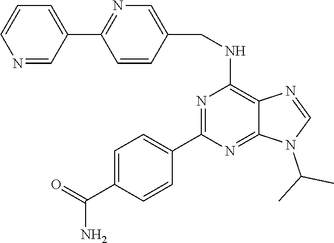 [SMILES: CC(C)n1cnc2c(NCc3ccc(-c4cccnc4)nc3)nc(-c3ccc(C(N)=O)cc3)nc21]

¹H NMR (400 MHz, DMSO-d₆) 59.22 (d, J=1.6 Hz, 1H), 8.84 (s, 1H), 8.60 (d, J=4.6 Hz, 2H), 8.45 (d, J=8.3 Hz, 2H), 8.39 (d, J=8.1 Hz, 1H), 8.34 (s, 1H), 8.03 (d, J=5.5 Hz, 1H), 7.99 (dd, J=10.8, 6.9 Hz, 4H), 7.49 (dd, J=7.9, 4.8 Hz, 1H), 7.43 (s, 1H), 4.87 (dd, J=13.6, 6.8 Hz, 3H), 1.60 (d, J=6.7 Hz, 6H). ¹³C NMR (101 MHz, DMSO-d₆) 5168.10, 156.73, 152.81, 150.17, 149.81, 148.13, 140.28, 137.04, 135.50, 134.39, 134.24, 128.00, 127.79, 124.21, 120.80, 47.06, 22.75; HRMS (ESI+) m/z calcd for C₂₆H₂₄N₈O [M+H]⁺: 465.2151, found: 465.2161.

### Example 11: N-([2,3′-bipyridin]-5-ylmethyl)-2-(1H-indol-5-yl)-9-isopropyl-9H-purin-6-amine

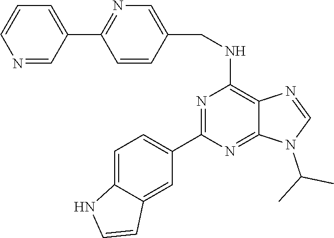 [SMILES: CC(C)n1cnc2c(NCc3ccc(-c4cccnc4)nc3)nc(-c3ccc4[nH]ccc4c3)nc21]

¹H NMR (400 MHz, CDCl₃) δ 9.19 (s, 1H), 8.85 (s, 2H), 8.65 (s, 1H), 8.50-8.25 (m, 3H), 7.93 (d, J=7.3 Hz, 1H), 7.82 (s, 1H), 7.70 (d, J=7.6 Hz, 1H), 7.54-7.34 (m, 2H), 7.27 (d, J=15.0 Hz, 2H), 6.68 (s, 1H), 6.40 (s, 1H), 5.13 (s, 2H), 5.00 (m, 1H), 1.69 (d, J=6.0 Hz, 6H). ¹³C NMR (101 MHz, CDCl₃) δ 159.96, 154.03, 153.73, 149.76, 149.64, 148.18, 137.57, 137.15, 136.60, 134.70, 134.52, 134.28, 128.02, 124.77, 123.57, 122.67, 121.27, 120.41, 110.63, 103.69, 46.92, 22.81; HRMS (ESI+) m/z calcd for C₂₇H₂₄N₈ [M+H]⁺: 461.2202, found: 461.2203.

### Example 12: N-([2,3′-bipyridin]-5-ylmethyl)-2-(1H-indol-6-yl)-9-isopropyl-9H-purin-6-amine

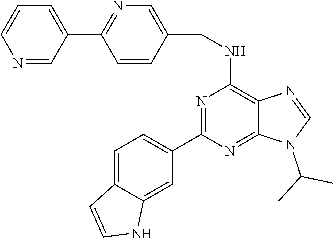 [SMILES: CC(C)n1cnc2c(NCc3ccc(-c4cccnc4)nc3)nc(-c3ccc4cc[nH]c4c3)nc21]

¹H NMR (400 MHz, CDCl₃) δ 9.20 (d, J=1.8 Hz, 1H), 8.90 (d, J=1.9 Hz, 1H), 8.69-8.55 (m, 2H), 8.32 (ddd, J=8.0, 5.0, 3.0 Hz, 2H), 7.95 (d, J=6.4 Hz, 1H), 7.89 (s, 1H), 7.72 (dd, J=8.3, 4.8 Hz, 2H), 7.41 (dd, J=8.0, 4.7 Hz, 1H), 7.33 (d, J=2.8 Hz, 1H), 6.61 (s, 1H), 6.48-6.19 (m, 1H), 5.15 (s, 2H), 5.01 (dt, J=13.6, 6.8 Hz, 1H), 1.71 (d, J=6.8 Hz, 6H).

### Example 13: N-([2,3′-bipyridin]-5-ylmethyl)-2-(1H-indazol-6-yl)-9-isopropyl-9H-purin-6-amine

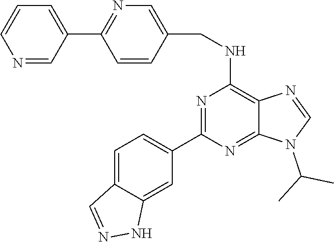 [SMILES: CC(C)n1cnc2c(NCc3ccc(-c4cccnc4)nc3)nc(-c3ccc4cn[nH]c4c3)nc21]

¹H NMR (400 MHz, CDCl₃) δ 9.20 (d, J=1.7 Hz, 1H), 8.91 (d, J=1.8 Hz, 1H), 8.75-8.62 (m, 2H), 8.34 (ddd, J=11.4, 8.0, 5.3 Hz, 2H), 8.13 (s, 1H), 7.99-7.90 (m, 2H), 7.86 (t, J=9.7 Hz, 1H), 7.74 (d, J=8.3 Hz, 1H), 7.42 (dd, J=7.9, 4.8 Hz, 1H), 6.53 (s, 1H), 5.14 (s, 2H), 5.01 (dt, J=13.5, 6.8 Hz, 1H), 1.72 (d, J=6.8 Hz, 6H). ¹³C NMR (101 MHz, CDCl₃+CD₃OD) δ 163.00, 157.18, 153.39, 153.00, 151.43, 142.03, 141.43, 141.03, 139.29, 139.04, 138.93, 127.99, 127.73, 125.17, 125.07, 124.08, 122.47, 51.22, 26.39; HRMS (ESI+) m/z calcd for C₂₆H₂₃N₉ [M+H]⁺: 462.2155, found: 462.2149.

### Example 14: N-([2,3′-bipyridin]-5-ylmethyl)-2-(1H-indazol-5-yl)-9-isopropyl-9H-purin-6-amine

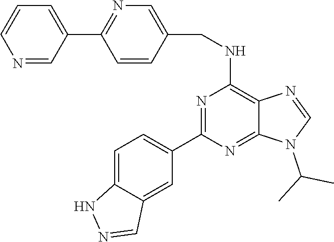 [SMILES: CC(C)n1cnc2c(NCc3ccc(-c4cccnc4)nc3)nc(-c3ccc4[nH]ncc4c3)nc21]

¹H NMR (400 MHz, DMSO-d₆) δ 13.14 (s, 1H), 9.22 (s, 1H), 8.84 (d, J=9.8 Hz, 2H), 8.60 (d, J=4.9 Hz, 1H), 8.48 (d, J=8.9 Hz, 2H), 8.38 (d, J=7.8 Hz, 1H), 8.26 (d, J=7.4 Hz, 1H), 8.21 (s, 1H), 8.02 (s, 2H), 7.59 (d, J=8.9 Hz, 1H), 7.52-7.45 (m, 1H), 4.88 (dd, J=13.6, 6.8 Hz, 3H), 1.61 (d, J=6.7 Hz, 7H). ¹³C NMR (101 MHz, CDCl₃+CD₃OD) δ 163.26, 157.87, 157.14, 153.30, 152.92, 151.39, 141.75, 140.94, 139.16, 139.03, 138.98, 135.88, 131.21, 127.98, 127.10, 125.12, 124.95, 122.12, 51.14, 26.41; HRMS (ESI+) m/z calcd for C₂₆H₂₃N₉ [M+H]⁺: 462.2155, found: 462.2154.

### Example 15: 5-(6-(([2,3′-bipyridin]-5-ylmethyl)amino)-9-isopropyl-9H-purin-2-yl)-1,3-dihydro-2H-benzo[d]imidazol-2-one

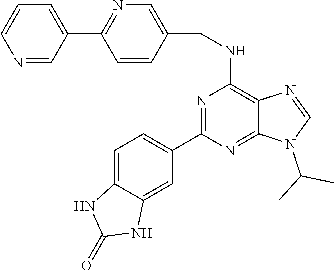 [SMILES: CC(C)n1cnc2c(NCc3ccc(-c4cccnc4)nc3)nc(-c3ccc4[nH]c(=O)[nH]c4c3)nc21]

¹H NMR (400 MHz, DMSO-d₆) 510.76 (s, 1H), 10.68 (s, 1H), 9.22 (s, 1H), 8.82 (s, 1H), 8.61 (d, J=3.8 Hz, 1H), 8.51-8.34 (m, 2H), 8.25 (s, 1H), 8.12 (d, J=8.2 Hz, 1H), 8.00 (dd, J=13.5, 5.6 Hz, 3H), 7.49 (dd, J=7.8, 4.8 Hz, 1H), 7.00 (d, J=8.2 Hz, 1H), 4.84 (dd, J=13.6, 6.8 Hz, 3H), 1.59 (d, J=6.6 Hz, 6H).

### Example 16: N-([2,3′-bipyridin]-5-ylmethyl)-9-isopropyl-2-(quinolin-3-yl)-9H-purin-6-amine

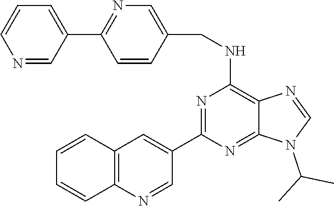 [SMILES: CC(C)n1cnc2c(NCc3ccc(-c4cccnc4)nc3)nc(-c3cnc4ccccc4c3)nc21]

¹H NMR (400 MHz, CDCl₃) δ 10.06 (d, J=1.9 Hz, 1H), 9.35 (s, 1H), 9.22 (d, J=2.0 Hz, 1H), 8.90 (s, 1H), 8.67 (d, J=3.3 Hz, 1H), 8.37 (d, J=8.1 Hz, 2H), 8.07 (d, J=8.5 Hz, 1H), 7.98 (dd, J=8.1, 2.1 Hz, 1H), 7.94 (s, 1H), 7.90-7.83 (m, 1H), 7.80 (d, J=8.2 Hz, 1H), 7.71 (d, J=7.5 Hz, 1H), 7.44 (dd, J=7.9, 4.8 Hz, 1H), 6.48 (s, 1H), 5.15 (s, 2H), 5.02 (dd, J=13.6, 7.0 Hz, 1H), 1.74 (d, J=6.8 Hz, 6H). ¹³C NMR (101 MHz, CDCl₃) δ 156.78, 153.96, 150.91, 149.89, 149.60, 148.62, 148.20, 138.42, 136.50, 135.24, 134.56, 134.26, 134.05, 131.39, 129.97, 129.28, 128.77, 127.79, 126.73, 123.58, 120.39, 47.33, 22.78; HRMS (ESI+) m/z calcd for C₂₈H₂₄N₈ [M+H]⁺ 473.2202, found: 473.2204.

### Example 17: N-([2,3′-bipyridin]-5-ylmethyl)-9-isopropyl-2-(6-methylpyridin-3-yl)-9H-purin-6-amine

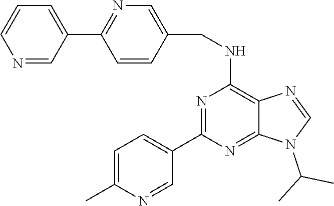 [SMILES: Cc1ccc(-c2nc(NCc3ccc(-c4cccnc4)nc3)c3ncn(C(C)C)c3n2)cn1]

¹H NMR (400 MHz, CDCl₃) δ 9.53 (s, TH), 9.15 (s, TH), 8.78 (d, J=1.6 Hz, 1H), 8.62 (s, 1H), 8.59 (dd, J=8.1, 2.0 Hz, 1H), 8.27 (d, J=8.0 Hz, 1H), 7.86 (dd, J=8.1, 2.1 Hz, 1H), 7.81 (s, 1H), 7.68 (d, J=8.1 Hz, 1H), 7.37 (dd, J=7.6, 4.7 Hz, 1H), 7.23 (d, J=8.1 Hz, 1H), 6.54 (s, 1H), 5.02 (s, 2H), 4.90 (dt, J=13.6, 6.8 Hz, 1H), 2.62 (s, 3H), 1.64 (d, J=6.8 Hz, 6H); ¹³C NMR (101 MHz, CDCl₃) δ 159.38, 156.99, 154.30, 153.94, 149.98, 149.63, 149.38, 148.30, 138.33, 136.56, 135.88, 134.65, 134.32, 134.22, 131.64, 123.66, 122.84, 120.40, 119.49, 77.48, 77.16, 76.84, 47.33, 41.94, 24.48, 22.80; HRMS (ESI+) m/z calcd for C₂₅H₂₄N₈ [M+H]⁺ 437.2202, found 437.2199.

### Example 18: N-([2,3′-bipyridin]-5-ylmethyl)-2-(6-aminopyridin-3-yl)-9-isopropyl-9H-purin-6-amine

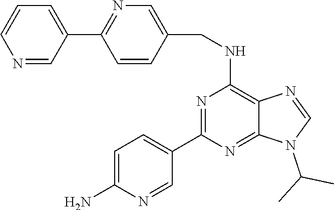 [SMILES: CC(C)n1cnc2c(NCc3ccc(-c4cccnc4)nc3)nc(-c3ccc(N)nc3)nc21]

¹H NMR (400 MHz, CDCl₃) δ 9.13 (dd, J=6.1, 1.8 Hz, 2H), 8.76 (d, J=1.7 Hz, 1H), 8.59 (dd, J=4.8, 1.6 Hz, 1H), 8.44 (dd, J=8.6, 2.2 Hz, 1H), 8.28-8.21 (m, 1H), 7.83 (dd, J=8.1, 2.2 Hz, 1H), 7.75 (s, J=13.0 Hz, 1H), 7.63 (d, J=8.1 Hz, 1H), 7.34 (dd, J=8.0, 4.8 Hz, 1H), 6.78 (s, 1H), 6.53 (d, J=8.6 Hz, 1H), 4.97 (d, J=4.0 Hz, 2H), 4.86 (dt, J=13.4, 6.7 Hz, 3H), 1.60 (t, J=6.0 Hz, 6H); ¹³C NMR (101 MHz, CDCl₃) δ 159.03, 157.38, 153.95, 149.97, 149.69, 148.55, 148.33, 138.02, 137.85, 136.61, 134.75, 134.38, 125.43, 123.70, 120.49, 119.04, 108.08, 77.48, 77.16, 76.84, 47.19, 22.85; HRMS (ESI+) m/z calcd for C₂₄H₂₃N₉ [M+H]⁺ 438.2155, found 438.2151.

### Example 19: N-([2,3′-bipyridin]-5-ylmethyl)-2-(2-aminopyridin-4-yl)-9-isopropyl-9H-purin-6-amine

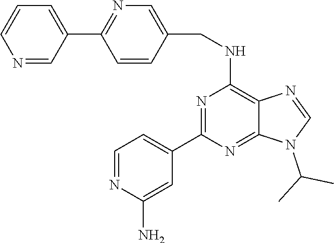 [SMILES: CC(C)n1cnc2c(NCc3ccc(-c4cccnc4)nc3)nc(-c3ccnc(N)c3)nc21]

¹H NMR (400 MHz, CDCl₃) δ 9.15 (d, J=1.7 Hz, 1H), 8.78 (d, J=1.7 Hz, 1H), 8.62 (dd, J=4.8, 1.5 Hz, 1H), 8.30-8.23 (m, 1H), 8.13 (d, J=5.4 Hz, 1H), 7.84 (s, 1H), 7.82 (d, J=2.2 Hz, 1H), 7.68 (s, 1H), 7.67-7.65 (m, 1H), 7.55 (s, 1H), 7.36 (dd, J=7.7, 5.1 Hz, 1H), 6.56 (s, 1H), 5.01 (s, 2H), 4.91 (dt, J=13.6, 6.8 Hz, 1H), 4.77 (s, 2H), 1.64 (d, J=6.8 Hz, 6H); ¹³C NMR (101 MHz, CDCl₃) δ 158.93, 156.67, 154.28, 154.04, 150.04, 149.75, 148.41, 148.30, 147.65, 138.80, 136.55, 134.62, 134.34, 134.16, 123.71, 120.50, 113.11, 107.70, 77.48, 77.16, 76.84, 47.39, 22.88; HRMS (ESI+) m/z calcd for C₂₄H₂₃N₉ [M+H]⁺ 438.2155, found 438.2163.

### Example 20: N-([2,3′-bipyridin]-5-ylmethyl)-9-isopropyl-2-(pyrimidin-5-yl)-9H-purin-6-amine

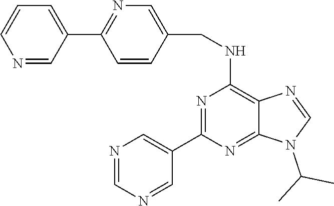 [SMILES: CC(C)n1cnc2c(NCc3ccc(-c4cccnc4)nc3)nc(-c3cncnc3)nc21]

¹H NMR (400 MHz, CDCl₃) δ 9.71 (s, 2H), 9.28 (s, 1H), 9.21 (d, J=1.6 Hz, 1H), 8.83 (d, J=1.7 Hz, 1H), 8.67 (dd, J=4.9, 1.6 Hz, 1H), 8.43-8.36 (m, 1H), 7.96-7.86 (m, 2H), 7.76 (d, J=8.1 Hz, 1H), 7.51-7.43 (m, 1H), 6.52 (s, 1H), 5.07 (s, 2H), 4.94 (dq, J=13.6, 6.8 Hz, 1H), 1.71 (t, J=5.6 Hz, 6H). ¹³C NMR (101 MHz, DMSO-d₆) δ 159.24, 156.22, 150.10, 149.80, 148.07, 140.60, 137.01, 135.69, 134.38, 134.30, 124.22, 120.80, 47.27, 22.68. HRMS (ESI) calcd for C₂₃H₂₁N₉ [M+H]⁺: 424.1998, found: 424.2002.

### Example 21: N-([2,3′-bipyridin]-5-ylmethyl)-2-(2-aminopyrimidin-5-yl)-9-isopropyl-9H-purin-6-amine

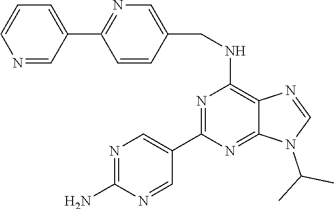 [SMILES: CC(C)n1cnc2c(NCc3ccc(-c4cccnc4)nc3)nc(-c3cnc(N)nc3)nc21]

¹H NMR (400 MHz, DMSO-d₆) δ 9.28 (s, 1H), 9.10 (s, 2H), 8.79 (s, 1H), 8.66 (s, 1H), 8.50 (s, 1H), 8.39 (d, J=7.5 Hz, 1H), 8.24 (s, 1H), 7.99 (d, J=8.1 Hz, 1H), 7.95 (d, J=7.7 Hz, 1H), 7.51 (s, 1H), 7.00 (s, 2H), 5.00-4.70 (m, 3H), 1.56 (d, J=6.7 Hz, 7H); ¹³C NMR (101 MHz, DMSO-d₆) δ 163.83, 157.59, 154.87, 152.26, 149.24, 147.49, 138.92, 136.44, 135.39, 133.74, 124.01, 120.69, 120.25, 46.38, 40.14, 39.94, 39.73, 39.52, 39.52, 39.31, 39.10, 38.90, 22.19; HRMS (ESI+) m/z calcd for C₂₃H₂₂N₁₀ [M+H]⁺ 439.2107, found 439.2113.

### Example 22: N-([2,3′-bipyridin]-5-ylmethyl)-9-isopropyl-2-(1H-pyrazol-5-yl)-9H-purin-6-amine

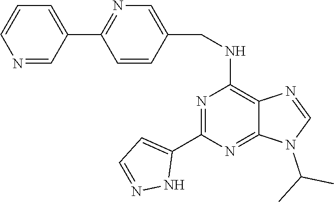 [SMILES: CC(C)n1cnc2c(NCc3ccc(-c4cccnc4)nc3)nc(-c3ccn[nH]3)nc21]

¹H NMR (400 MHz, CDCl₃) δ 9.09 (d, J=1.6 Hz, 1H), 8.71 (d, J=1.7 Hz, 1H), 8.57 (d, J=3.5 Hz, 1H), 8.28-8.15 (m, 1H), 7.82-7.74 (m, 2H), 7.66-7.51 (m, 2H), 7.32 (dd, J=7.9, 4.8 Hz, 1H), 6.92 (d, J=1.4 Hz, 1H), 6.59 (s, 1H), 4.92 (s, 1H), 4.82 (dd, J=13.6, 6.8 Hz, 1H), 1.56 (d, J=6.8 Hz, 5H). ¹³C NMR (101 MHz, CDCl₃) δ153.98, 149.92, 149.59, 148.19, 138.14, 136.47, 134.52, 134.27, 133.81, 123.60, 120.37, 105.52, 47.08, 22.79; HRMS (ESI+) m/z calcd for C₂₂H₂₁N₉ [M+H]⁺: 412.1998, found: 412.2005.

### Example 23: N-([2,3′-bipyridin]-5-ylmethyl)-9-isopropyl-2-(1H-pyrazol-4-yl)-9H-purin-6-amine

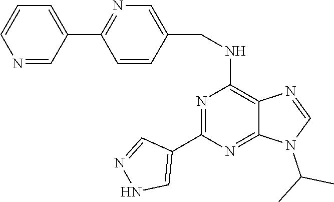 [SMILES: CC(C)n1cnc2c(NCc3ccc(-c4cccnc4)nc3)nc(-c3cn[nH]c3)nc21]

¹H NMR (400 MHz, CDCl₃) δ 9.02 (d, J=6.3 Hz, 1H), 8.67 (d, J=6.6 Hz, 1H), 8.48 (d, J=4.8 Hz, 1H), 8.20-7.97 (m, 3H), 7.79 (d, J=8.0 Hz, 1H), 7.68 (s, 1H), 7.57 (t, J=7.9 Hz, 1H), 7.28-7.22 (m, 2H), 6.77 (s, 1H), 4.85 (s, 2H), 4.74 (dd, J=13.9, 6.8 Hz, 1H), 1.48 (t, J=7.6 Hz, 6H). ¹³C NMR (101 MHz, CDCl₃+CD₃OD) 5155.57, 153.33, 149.54, 149.27, 147.69, 137.13, 136.78, 134.87, 134.78, 123.86, 123.48, 120.71, 117.87, 46.85, 22.68; HRMS (ESI+) m/z calcd for C₂₂H₂₁N₉ [M+H]⁺: 412.1998, found: 412.2001.

### Example 24: N-([2,3′-bipyridin]-5-ylmethyl)-9-isopropyl-2-(thiazol-5-yl)-9H-purin-6-amine

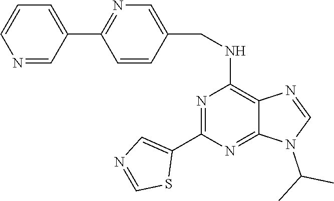 [SMILES: CC(C)n1cnc2c(NCc3ccc(-c4cccnc4)nc3)nc(-c3cncs3)nc21]

¹H NMR (400 MHz, CDCl₃) δ 9.10 (d, J=1.7 Hz, 1H), 8.78-8.70 (m, 2H), 8.60-8.51 (m, 2H), 8.28-8.20 (m, 1H), 7.83 (dt, J=10.0, 5.0 Hz, 1H), 7.74 (d, J=9.6 Hz, 1H), 7.66 (t, J=7.0 Hz, 1H), 7.33 (dd, J=7.7, 5.0 Hz, 1H), 6.35 (s, 1H), 4.91 (s, 2H), 4.83-4.75 (m, 1H), 1.57 (d, J=6.8 Hz, 6H).

### Example 25: N-([2,3′-bipyridin]-5-ylmethyl)-2-(2-aminophenyl)-9-isopropyl-9H-purin-6-amine

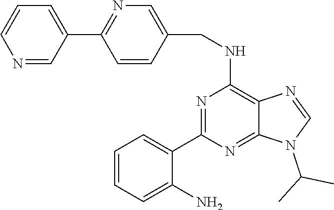 [SMILES: CC(C)n1cnc2c(NCc3ccc(-c4cccnc4)nc3)nc(-c3ccccc3N)nc21]

¹H NMR (400 MHz, CDCl₃) δ 9.20 (d, J=1.7 Hz, 1H), 8.83 (s, 1H), 8.70-8.64 (m, 1H), 8.47 (dd, J=8.0, 1.4 Hz, 1H), 8.34 (d, J=8.0 Hz, 1H), 7.92 (d, J=8.1 Hz, 1H), 7.86 (s, 1H), 7.74 (d, J=8.1 Hz, 1H), 7.43 (dd, J=7.9, 4.8 Hz, 1H), 7.24-7.17 (m, 1H), 6.80 (t, J=7.2 Hz, 1H), 6.75 (d, J=8.0 Hz, 1H), 6.45 (s, 1H), 5.05 (s, 2H), 4.89 (dt, J=13.5, 6.8 Hz, 1H), 1.68 (d, J=6.8 Hz, 6H). ¹³C NMR (101 MHz, CDCl₃) δ160.21, 153.95, 153.62, 149.91, 149.44, 148.22, 147.90, 137.85, 136.34, 134.55, 134.24, 133.93, 131.22, 130.75, 123.58, 120.44, 120.39, 117.13, 117.03, 47.17, 22.69; HRMS (ESI+) m/z calcd for C₂₅H₂₄N₈ [M+H]⁺: 437.2202, found: 437.2204.

### Example 26: N-([2,3′-bipyridin]-5-ylmethyl)-2-(3-aminophenyl)-9-isopropyl-9H-purin-6-amine

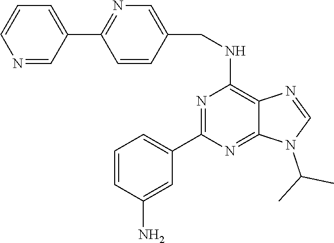 [SMILES: CC(C)n1cnc2c(NCc3ccc(-c4cccnc4)nc3)nc(-c3cccc(N)c3)nc21]

¹H NMR (600 MHz, CDCl₃) δ 9.16 (s, 1H), 8.82 (s, 1H), 8.63 (d, J=4.2 Hz, 1H), 8.29 (d, J=7.8 Hz, 1H), 7.96-7.78 (m, 3H), 7.70 (d, J=8.2 Hz, 1H), 7.45-7.35 (m, 1H), 7.24 (d, J=12.2 Hz, 2H), 6.77 (d, J=6.9 Hz, 1H), 6.36 (s, 1H), 5.17-4.88 (m, 3H), 1.65 (d, J=6.7 Hz, 6H). ¹³C NMR (101 MHz, DMSO-d₆) δ 158.26, 152.71, 149.91, 148.90, 147.91, 139.71, 137.15, 136.08, 134.44, 129.07, 124.30, 122.58, 120.81, 120.59, 116.37, 115.88, 113.92, 46.94, 22.73; HRMS (ESI+) m/z calcd for C₂₅H₂₄N₈ [M+H]⁺: 437.2202, found: 437.2205.

### Example 27: N-([2,3′-bipyridin]-5-ylmethyl)-2-(4-aminophenyl)-9-isopropyl-9H-purin-6-amine

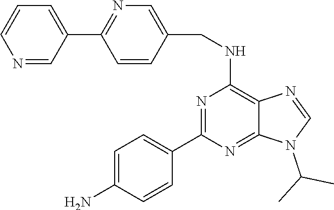 [SMILES: CC(C)n1cnc2c(NCc3ccc(-c4cccnc4)nc3)nc(-c3ccc(N)cc3)nc21]

¹H NMR (400 MHz, CDCl₃) δ 9.19 (d, J=2.0 Hz, 1H), 8.84 (d, J=1.7 Hz, 1H), 8.66 (dd, J=4.8, 1.5 Hz, 1H), 8.37-8.28 (m, 3H), 7.92 (dd, J=8.1, 2.1 Hz, 1H), 7.80 (s, 1H), 7.71 (d, J=8.2 Hz, 1H), 7.41 (dd, J=8.0, 4.8 Hz, 1H), 6.76 (d, J=8.6 Hz, 2H), 6.37 (s, 1H), 5.08 (s, 2H), 4.95 (dq, J=13.5, 6.6 Hz, 1H), 3.87 (s, 2H), 1.67 (d, J=6.8 Hz, 6H). ¹³C NMR (101 MHz, CDCl₃) δ 153.81, 149.82, 149.65, 148.21, 137.53, 136.56, 134.69, 134.40, 134.27, 129.64, 123.57, 120.41, 114.55, 46.91, 22.76; HRMS (ESI+) m/z calcd for C₂₅H₂₄N₈ [M+H]⁺: 437.2202, found: 437.2199.

### Example 28: N-([2,3′-bipyridin]-5-ylmethyl)-2-(6-fluoropyridin-3-yl)-9-isopropyl-9H-purin-6-amine

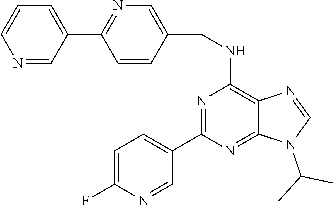 [SMILES: CC(C)n1cnc2c(NCc3ccc(-c4cccnc4)nc3)nc(-c3ccc(F)nc3)nc21]

¹H NMR (400 MHz, CDCl₃) δ 9.26 (d, J=2.3 Hz, 1H), 9.15 (s, 1H), 8.78 (dd, J=8.3, 2.4 Hz, 1H), 8.75 (d, J=2.3 Hz, 1H), 8.62 (s, 1H), 8.29-8.23 (m, 1H), 7.82 (dd, J=8.2, 2.3 Hz, 1H), 7.79 (s, 1H), 7.66 (d, J=8.0 Hz, 1H), 7.35 (dd, J=7.8, 4.8 Hz, 1H), 6.96 (dd, J=8.6, 2.7 Hz, 1H), 6.71 (s, 1H), 5.00 (s, 2H), 4.88 (dt, J=13.5, 6.8 Hz, 1H), 1.63 (d, J=6.8 Hz, 6H); ¹³C NMR (101 MHz, CDCl₃) δ 165.72, 163.33, 155.86, 154.36, 154.02, 150.02, 149.59, 148.38, 148.27, 148.23, 140.96, 140.88, 138.50, 136.47, 134.29, 134.05, 132.63, 132.59, 123.67, 120.40, 119.55, 109.06, 108.69, 77.48, 77.16, 76.84, 47.41, 41.92, 22.80; HRMS (ESI+) m/z calcd for C₂₄H₂₁FN₈ [M+H]⁺ 441.1951, found 441.1944.

### Example 29: N-([2,3′-bipyridin]-5-ylmethyl)-2-(2-fluoropyridin-4-yl)-9-isopropyl-9H-purin-6-amine

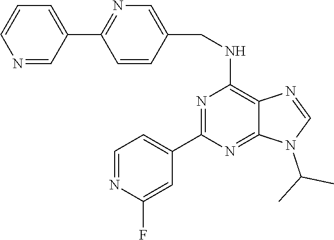 [SMILES: CC(C)n1cnc2c(NCc3ccc(-c4cccnc4)nc3)nc(-c3ccnc(F)c3)nc21]

¹H NMR (400 MHz, CDCl₃) δ 9.15 (d, J=1.8 Hz, 1H), 8.78 (d, J=1.7 Hz, 1H), 8.63 (dd, J=4.8, 1.4 Hz, 1H), 8.28 (d, J=4.6 Hz, 1H), 8.28-8.25 (m, 1H), 8.18 (d, J=5.2 Hz, 1H), 7.92 (s, 1H), 7.86 (s, 1H), 7.86-7.81 (m, 1H), 7.70 (d, J=8.1 Hz, 1H), 7.37 (dd, J=8.0, 4.8 Hz, 1H), 6.62 (s, 1H), 5.03 (s, 2H), 4.91 (dt, J=13.6, 6.8 Hz, 1H), 1.66 (d, J=6.8 Hz, 6H); ¹³C NMR (101 MHz, CDCl₃) δ 165.95, 163.60, 155.33, 154.37, 154.18, 152.12, 150.10, 149.66, 148.32, 147.88, 147.73, 139.23, 136.53, 134.57, 134.34, 133.89, 123.70, 120.48, 120.12, 120.08, 108.31, 107.93, 77.48, 77.16, 76.84, 47.60, 22.85; HRMS (ESI+) m/z calcd for C₂₄H₂₁FN₈ [M+H]⁺ 441.1951, found 441.1954

### Example 30: N-([2,3′-bipyridin]-5-ylmethyl)-2-(2-fluoropyridin-3-yl)-9-isopropyl-9H-purin-6-amine

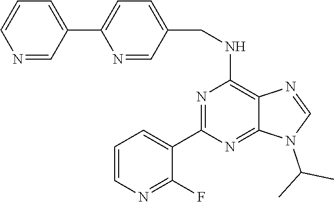 [SMILES: CC(C)n1cnc2c(NCc3ccc(-c4cccnc4)nc3)nc(-c3cccnc3F)nc21]

¹H NMR (400 MHz, CDCl₃) δ 9.20 (d, J=1.8 Hz, 1H), 8.83 (d, J=1.8 Hz, 1H), 8.70-8.52 (m, 2H), 8.34 (ddd, J=19.1, 10.5, 3.3 Hz, 2H), 7.99-7.87 (m, 2H), 7.75 (d, J=8.1 Hz, 1H), 7.44 (dd, J=7.9, 4.9 Hz, 1H), 7.35-7.30 (m, 1H), 6.47 (s, 1H), 5.05 (s, 2H), 4.94 (dt, J=13.5, 6.8 Hz, 1H), 1.69 (d, J=6.8 Hz, 6H). ¹³C NMR (101 MHz, CDCl₃) δ 162.67, 160.22, 155.23, 154.21, 153.85, 149.87, 149.65, 148.19, 147.88, 147.74, 142.24, 142.21, 138.57, 136.66, 134.56, 134.23, 134.05, 123.57, 122.55, 121.28, 121.23, 120.33, 47.37, 22.68.

### Example 31: 2-((2-(6-(([2,3′-bipyridin]-5-ylmethyl)amino)-9-isopropyl-9H-purin-2-yl)phenyl)amino)ethan-1-ol

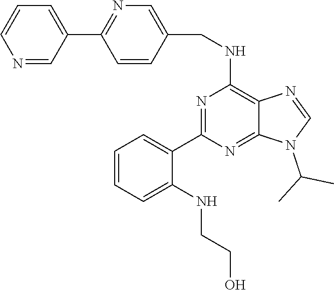 [SMILES: CC(C)n1cnc2c(NCc3ccc(-c4cccnc4)nc3)nc(-c3ccccc3NCCO)nc21]

¹H NMR (600 MHz, CDCl₃) δ9.09 (d, J=1.5 Hz, 1H), 8.67 (s, 1H), 8.58 (d, J=3.8 Hz, 1H), 8.44 (t, J=12.6 Hz, 1H), 8.21 (d, J=8.0 Hz, 1H), 7.74 (d, J=8.6 Hz, 1H), 7.71 (s, 1H), 7.56 (d, J=8.1 Hz, 1H), 7.32 (dd, J=7.9, 4.8 Hz, 1H), 7.28-7.23 (m, 2H), 6.76-6.70 (m, 2H), 6.64 (d, J=34.1 Hz, 1H), 4.91 (s, 2H), 4.86-4.81 (m, 1H), 3.89 (t, J=5.0 Hz, 2H), 3.41 (t, J=5.0 Hz, 2H), 1.59 (t, J=11.7 Hz, 6H). ¹³C NMR (101 MHz, CDCl₃) δ 160.14, 153.76, 153.48, 149.76, 149.44, 148.72, 148.12, 137.58, 136.35, 134.54, 134.26, 133.94, 131.54, 131.17, 123.57, 120.31, 115.76, 111.48, 61.31, 47.07, 45.91, 22.68; HRMS (ESI+) m/z calcd for C₂₇H₂₈N₈O [M+H]⁺: 481.2464, found: 481.2467.

### Example 32: 3-((2-(6-(([2,3′-bipyridin]-5-ylmethyl)amino)-9-isopropyl-9H-purin-2-yl)phenyl)amino)propan-1-ol

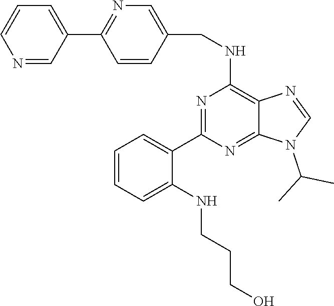 [SMILES: CC(C)n1cnc2c(NCc3ccc(-c4cccnc4)nc3)nc(-c3ccccc3NCCCO)nc21]

¹H NMR (600 MHz, CDCl₃) δ 9.14 (s, 1H), 8.81 (d, J=52.8 Hz, 2H), 8.61 (s, 1H), 8.50 (d, J=7.0 Hz, 1H), 8.26 (d, J=7.5 Hz, 1H), 7.91-7.75 (m, 2H), 7.66 (d, J=7.6 Hz, 1H), 7.36 (s, 1H), 7.26 (d, J=11.8 Hz, 1H), 6.74 (dd, J=20.9, 7.4 Hz, 2H), 6.51 (s, 1H), 5.01 (s, 2H), 4.84 (m, 1H), 3.79 (t, 2H), 3.37 (t, 2H), 1.93 (m, 2H), 1.63 (d, J=6.3 Hz, 6H). ¹³C NMR (101 MHz, CDCl₃) δ 153.51, 149.79, 149.43, 148.11, 137.65, 136.39, 134.24, 134.00, 131.44, 131.25, 120.35, 77.36, 77.04, 76.73, 61.17, 47.11, 46.03, 22.68, 8.60; HRMS (ESI+) m/z calcd for C₂₈H₃₀N₈O [M+H]⁺: 495.2621, found: 495.2626.

### Example 33: 3-((3-(6-(([2,3′-bipyridin]-5-ylmethyl)amino)-9-isopropyl-9H-purin-2-yl)phenyl)amino)propan-1-ol

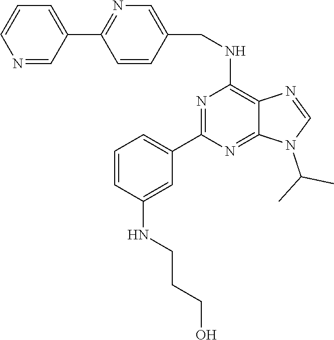 [SMILES: CC(C)n1cnc2c(NCc3ccc(-c4cccnc4)nc3)nc(-c3cccc(NCCCO)c3)nc21]

¹H NMR (600 MHz, CDCl₃) δ 9.12 (s, 1H), 8.82 (d, J=18.5 Hz, 1H), 8.61 (d, J=4.2 Hz, 1H), 8.25 (t, J=18.7 Hz, 1H), 7.83 (dd, J=27.7, 7.8 Hz, 3H), 7.70-7.57 (m, 2H), 7.37 (dd, J=7.7, 4.9 Hz, 1H), 7.28-7.14 (m, 2H), 6.69 (d, J=7.6 Hz, 1H), 6.63 (s, 1H), 4.97 (s, 2H), 4.91 (dt, J=13.4, 6.7 Hz, 1H), 3.79 (t, J=5.7 Hz, 2H), 3.33 (t, J=6.5 Hz, 2H), 1.94-1.85 (m, 2H), 1.63 (t, J=9.0 Hz, 6H). ¹³C NMR (201 MHz, CDCl₃) δ149.85, 149.62, 148.23, 136.50, 134.40, 120.75, 117.76, 96.16, 61.43, 42.39, 32.23, 22.81; HRMS (ESI+) m/z calcd for C₂₈H₃₀N₈O [M+H]⁺: 495.2621, found: 495.2625.

### Example 34: 3-((4-(6-(([2,3′-bipyridin]-5-ylmethyl)amino)-9-isopropyl-9H-purin-2-yl)phenyl)amino)propan-1-ol

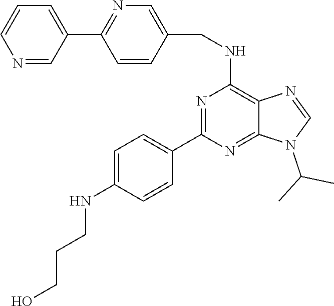 [SMILES: CC(C)n1cnc2c(NCc3ccc(-c4cccnc4)nc3)nc(-c3ccc(NCCCO)cc3)nc21]

¹H NMR (400 MHz, CDCl₃) δ 9.18 (d, J=1.8 Hz, 1H), 8.84 (s, 1H), 8.65 (dd, J=4.7, 1.3 Hz, 1H), 8.39-8.26 (m, 3H), 7.92 (dd, J=8.2, 1.9 Hz, 1H), 7.79 (s, 1H), 7.70 (d, J=8.1 Hz, 1H), 7.40 (dd, J=7.9, 4.8 Hz, 1H), 6.69 (d, J=8.7 Hz, 2H), 6.42 (s, 1H), 5.07 (s, 2H), 4.94 (dt, J=13.5, 6.7 Hz, 1H), 3.85 (t, J=5.9 Hz, 2H), 3.38 (t, J=6.5 Hz, 2H), 1.93 (dt, J=12.3, 6.2 Hz, 2H), 1.67 (t, J=9.1 Hz, 6H). ¹³C NMR (101 MHz, CDCl₃) δ 153.78, 149.80, 149.66, 148.22, 136.56, 134.28, 129.59, 123.58, 120.42, 112.31, 61.36, 46.74, 41.44, 31.93, 22.76; HRMS (ESI+) m/z calcd for C₂₈H₃₀N₈O [M+H]⁺: 495.2621, found: 495.2630.

### Example 35: 2-((5-(6-(([2,3′-bipyridin]-5-ylmethyl)amino)-9-isopropyl-9H-purin-2-yl)pyridin-2-yl)amino)ethan-1-ol

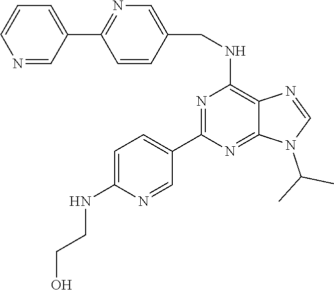 [SMILES: CC(C)n1cnc2c(NCc3ccc(-c4cccnc4)nc3)nc(-c3ccc(NCCO)nc3)nc21]

¹H NMR (400 MHz, CDCl₃) δ 9.14 (dd, J=6.8, 1.8 Hz, 2H), 8.76 (d, J=1.8 Hz, 1H), 8.61 (dd, J=4.7, 1.3 Hz, 1H), 8.42 (dd, J=8.8, 2.2 Hz, 1H), 8.29-8.23 (m, 1H), 7.82 (dd, J=8.1, 2.2 Hz, 1H), 7.76 (s, 1H), 7.66 (d, J=8.1 Hz, 1H), 7.36 (dd, J=7.6, 4.8 Hz, 1H), 6.48 (d, J=8.7 Hz, 1H), 6.40 (s, 1H), 5.22 (s, 1H), 4.99 (d, J=4.7 Hz, 2H), 4.86 (dt, J=13.6, 6.8 Hz, 1H), 3.88-3.82 (m, 2H), 3.58 (dd, J=9.4, 5.1 Hz, 2H), 1.63 (d, J=6.8 Hz, 6H); ¹³C NMR (101 MHz, CDCl₃) δ 159.46, 157.51, 154.15, 153.87, 149.90, 149.65, 148.41, 148.28, 137.77, 137.48, 136.57, 134.75, 134.39, 124.54, 123.70, 120.48, 118.90, 107.73, 77.48, 77.16, 76.84, 63.44, 47.23, 45.59, 22.80; HRMS (ESI+) m/z calcd for C₂₆H₂₇N₉O [M+H]⁺ 482.2417, found 482.2431.

### Example 36: 3-((5-(6-(([2,3′-bipyridin]-5-ylmethyl)amino)-9-isopropyl-9H-purin-2-yl)pyridin-2-yl)amino)propan-1-ol

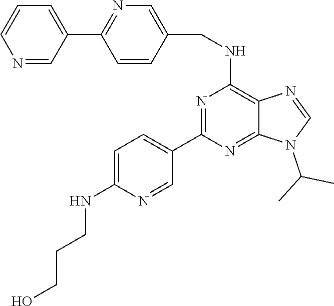 [SMILES: CC(C)n1cnc2c(NCc3ccc(-c4cccnc4)nc3)nc(-c3ccc(NCCCO)nc3)nc21]

¹H NMR (400 MHz, CDCl₃) δ 9.08 (s, 2H), 8.71 (d, J=1.8 Hz, 1H), 8.55 (dd, J=4.8, 1.5 Hz, 1H), 8.33 (dd, J=8.8, 2.2 Hz, 1H), 8.24-8.17 (m, 1H), 7.78 (dd, J=8.1, 2.2 Hz, 1H), 7.69 (s, 1H), 7.61 (d, J=8.1 Hz, 1H), 7.30 (dd, J=8.0, 4.8 Hz, 1H), 6.37 (d, J=8.8 Hz, 2H), 4.91 (dd, J=16.3, 5.8 Hz, 3H), 4.78 (dt, J=13.7, 6.8 Hz, 1H), 3.59 (t, J=5.6 Hz, 2H), 3.56-3.51 (m, 2H), 1.75-1.67 (m, 2H), 1.56 (d, J=6.8 Hz, 6H). ¹³C NMR (101 MHz, CDCl₃) δ 163.42, 161.69, 157.13, 153.23, 152.96, 151.98, 151.44, 141.40, 140.84, 138.97, 127.94, 127.46, 124.85, 111.06, 63.04, 51.07, 42.42, 36.17, 26.40; HRMS (ESI+) m/z calcd for C₂₇H₂₉N₉O [M+H]⁺: 496.2573, found: 496.2570.

### Example 37: 2-((4-(6-(([2,3′-bipyridin]-5-ylmethyl)amino)-9-isopropyl-9H-purin-2-yl)pyridin-2-yl)amino)ethan-1-ol

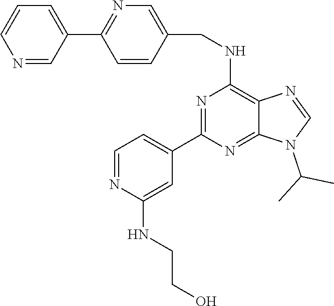 [SMILES: CC(C)n1cnc2c(NCc3ccc(-c4cccnc4)nc3)nc(-c3ccnc(NCCO)c3)nc21]

¹H NMR (400 MHz, CDCl₃) δ 9.15 (d, J=1.7 Hz, 1H), 8.82 (d, J=1.5 Hz, 1H), 8.63 (d, J=3.4 Hz, 1H), 8.28 (d, J=8.0 Hz, 1H), 8.10 (d, J=5.5 Hz, 1H), 7.89 (s, 1H), 7.86 (dd, J=8.2, 2.1 Hz, 1H), 7.69 (d, J=8.1 Hz, 1H), 7.62 (d, J=4.6 Hz, 1H), 7.49 (s, 1H), 7.38 (dd, J=7.9, 4.8 Hz, 1H), 6.49 (s, 1H), 4.99 (s, 2H), 4.91 (dt, J=13.4, 6.8 Hz, 1H), 3.83 (t, J=4.8 Hz, 2H), 3.57 (d, J=3.8 Hz, 2H), 1.65 (d, J=6.8 Hz, 6H); ¹³C NMR (101 MHz, CDCl₃) δ 159.45, 156.70, 154.09, 153.88, 149.93, 149.61, 148.17, 147.74, 147.40, 138.67, 136.43, 134.45, 134.23, 134.14, 123.60, 120.46, 112.11, 107.23, 77.35, 77.03, 76.71, 63.55, 47.28, 45.72, 22.74; HRMS (ESI+) m/z calcd for C₂₆H₂₇N₉O [M+H]⁺ 482.2417, found 482.2420.

### Example 38: 3-((4-(6-(([2,3′-bipyridin]-5-ylmethyl)amino)-9-isopropyl-9H-purin-2-yl)pyridin-2-yl)amino)propan-1-ol

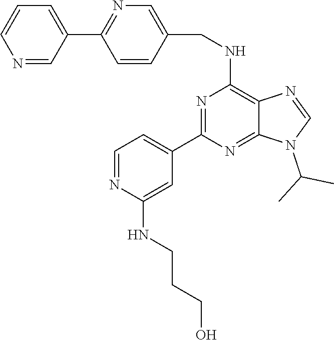 [SMILES: CC(C)n1cnc2c(NCc3ccc(-c4cccnc4)nc3)nc(-c3ccnc(NCCCO)c3)nc21]

¹H NMR (400 MHz, CDCl₃) δ 9.13 (s, 1H), 8.78 (s, 1H), 8.61 (d, J=4.1 Hz, 1H), 8.23 (d, J=7.9 Hz, 1H), 8.09 (d, J=5.4 Hz, 1H), 7.83 (s, 1H), 7.81 (d, J=8.1 Hz, 1H), 7.64 (d, J=8.1 Hz, 1H), 7.54 (d, J=5.4 Hz, 1H), 7.39 (s, 1H), 7.35 (dd, J=7.8, 4.8 Hz, 1H), 6.76 (s, 1H), 4.96 (s, 2H), 4.89 (dt, J=13.4, 6.9 Hz, 1H), 3.67-3.63 (m, 2H), 3.56 (d, J=5.5 Hz, 2H), 1.80-1.73 (m, 2H), 1.62 (d, J=6.8 Hz, 6H); ¹³C NMR (101 MHz, CDCl₃) δ 159.21, 156.56, 154.20, 154.01, 150.06, 149.70, 148.29, 146.31, 138.87, 136.50, 134.60, 134.37, 134.30, 123.74, 120.64, 111.58, 107.36, 77.48, 77.16, 76.84, 58.93, 47.43, 38.60, 33.48, 22.88.

### Example 39: 3-((3-(6-(([2,3′-bipyridin]-5-ylmethyl)amino)-9-isopropyl-9H-purin-2-yl)pyridin-2-yl)amino)propan-1-ol

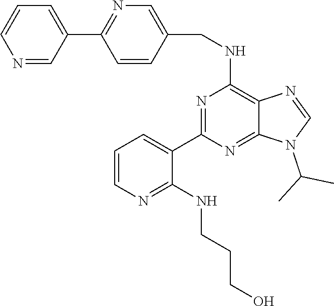 [SMILES: CC(C)n1cnc2c(NCc3ccc(-c4cccnc4)nc3)nc(-c3cccnc3NCCCO)nc21]

¹H NMR (400 MHz, CDCl₃) δ 9.63 (s, 1H), 9.19 (d, J=1.9 Hz, 1H), 8.88-8.73 (m, 2H), 8.67 (dd, J=4.8, 1.5 Hz, 1H), 8.38-8.26 (m, 1H), 8.14 (dd, J=4.9, 1.9 Hz, 1H), 7.95-7.82 (m, 2H), 7.75 (d, J=8.2 Hz, 1H), 7.42 (dd, J=8.0, 4.8 Hz, 1H), 6.66 (dd, J=7.7, 4.9 Hz, 1H), 6.43 (s, 1H), 5.05 (s, 2H), 4.86 (dt, J=13.6, 6.8 Hz, 1H), 3.70 (dd, J=11.8, 6.1 Hz, 2H), 3.63 (t, J=5.5 Hz, 2H), 1.71 (d, J=6.8 Hz, 6H).

Synthesis Method of Intermediate N-([2,3′-bipyridin]-5-ylmethyl)-2-hydrazineyl-9-isopropyl-9H-purin-6-amine (VI)

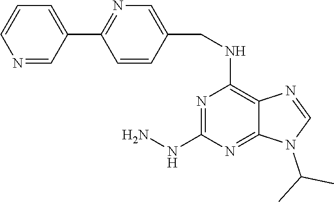 [SMILES: CC(C)n1cnc2c(NCc3ccc(-c4cccnc4)nc3)nc(NN)nc21]

Intermediate Vf (600 mg) and NH₂NH₂·H₂O (0.5 mL) were mixed in n-butanol (1 mL) at room temperature and then heated to 150° C. with stirring overnight. After the mixture was cooled to room temperature, water (10 mL) was added thereto and the pure solid (300 mg) was filtered (a yield of 48%). ¹H NMR (400 MHz, DMSO-d₆) δ 9.23 (d, J=1.6 Hz, 1H), 8.76 (s, 1H), 8.62 (dd, J=4.7, 1.6 Hz, 1H), 8.45-8.36 (m, 1H), 8.11-7.83 (m, 4H), 7.57-7.46 (m, 1H), 7.40 (s, 1H), 4.80-4.49 (m, 3H), 4.05 (s, 2H), 1.48 (d, J=6.8 Hz, 6H). ¹³C NMR (101 MHz, DMSO-d₆) δ 161.99, 152.64, 150.13, 148.14, 134.48, 134.23, 124.22, 120.67, 46.05, 22.64.

### Example 40: N-([2,3′-bipyridin]-5-ylmethyl)-2-(5-amino-3-methyl-1H-pyrazol-1-yl)-9-isopropyl-9H-purin-6-amine

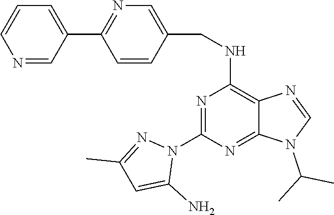 [SMILES: Cc1cc(N)n(-c2nc(NCc3ccc(-c4cccnc4)nc3)c3ncn(C(C)C)c3n2)n1]

Intermediate VI (40 mg, 1 eq) and 3-oxobutanenitrile (1.5 eq) were mixed in ethanol (2 mL) at room temperature, and then the mixture was refluxed with stirring. After the reaction was completed, ethanol was evaporated and a compound was obtained through column chromatography using 2% methanol in dichloromethane. Yield 26%; ¹H NMR (400 MHz, CDCl₃) δ 9.20 (s, 1H), 8.79 (s, 1H), 8.67 (d, J=4.8 Hz, 1H), 8.40-8.28 (m, 1H), 7.90 (d, J=6.1 Hz, 1H), 7.83 (s, 1H), 7.74 (d, J=8.1 Hz, 1H), 7.43 (dd, J=7.8, 4.8 Hz, 1H), 6.54 (s, 1H), 5.40 (s, 1H), 4.98 (s, 3H), 2.30 (d, J=6.1 Hz, 3H), 1.93 (d, J=70.3 Hz, 2H), 1.66-1.56 (m, 6H). ¹³C NMR (101 MHz, CDCl₃) δ 154.10, 151.38, 149.96, 149.39, 149.25, 148.20, 137.55, 136.34, 134.22, 123.54, 120.32, 90.29, 46.44, 23.05, 14.42; HRMS (ESI+) m/z calcd for C₂₃H₂₄N₁₀ [M+H]⁺: 441.2264, found: 441.2270.

Synthesis Method of Intermediate N-([2,3′-bipyridin]-5-ylmethyl)-2-azido-9-isopropyl-9H-purin-6-amine (VII)

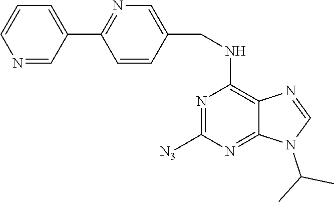 [SMILES: CC(C)n1cnc2c(NCc3ccc(-c4cccnc4)nc3)nc(N=[N+]=[N-])nc21]

Intermediate VI (300 mg) and NaNO₂ (250 mg, 1.5 eq)/HCl (1 mL) were mixed in water (4 mL) at 4° C., the resulting mixture was stirred for 30 minutes, and then a solution of NaN₃ (150 mg, 1.2 eq) dissolved in 2 mL of water was added dropwise, and the resulting mixture was stirred at room temperature for 12 hours. The reaction mixture was diluted with water (100 mL) and extracted with ethyl acetate (100 mL). The organic layer was washed with brine, dried over MgSO₄, concentrated under reduced pressure, and purified by column chromatography (1% methanol in dichloromethane) to obtain a product (200 mg, yield: 40%). ¹H NMR (400 MHz, CDCl₃) δ 9.10 (s, 1H), 8.66 (d, J=1.5 Hz, 1H), 8.57 (s, 1H), 8.29-8.14 (m, 1H), 7.75 (dd, J=8.1, 2.2 Hz, 1H), 7.65-7.54 (m, 2H), 7.32 (dd, J=7.8, 4.8 Hz, 1H), 6.95 (d, J=6.6 Hz, 1H), 4.80 (s, 2H), 4.72-4.56 (m, 1H), 1.48 (d, J=6.7, 6H). ¹³C NMR (101 MHz, CDCl₃) δ 156.38, 153.95, 149.91, 149.68, 148.18, 137.38, 136.65, 134.52, 134.27, 133.55, 123.61, 120.30, 77.39, 77.27, 77.07, 76.75, 47.06, 22.75, 22.62.

### Synthesis Method of Examples 40 and 41

2-Propyn-1-ol or 3-butyn-1-ol (0.24 mmol) was added to a mixture of t-butanol (2 mL) and water (2 mL) in which Intermediate VII (0.171 mmol) was dissolved. A freshly prepared 1 M sodium ascorbate solution (174 μL, 0.15 mmol) and a 7.5% CuSO₄·5H₂O solution (288 μL, 0.06 mmol) were added to the reaction mixture and stirred at room temperature overnight. The solvent was evaporated and the residue was purified by column chromatography using 2% methanol in dichloromethane to obtain a pure compound.

### Example 41: (1-(6-(([2,3′-bipyridin]-5-ylmethyl)amino)-9-isopropyl-9H-purin-2-yl)-1H-1,2,3-triazol-4-yl)methanol

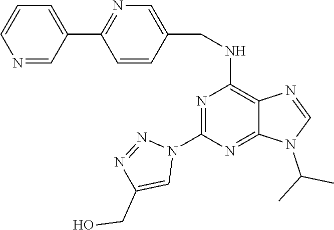 [SMILES: CC(C)n1cnc2c(NCc3ccc(-c4cccnc4)nc3)nc(-n3cc(CO)nn3)nc21]

¹H NMR (400 MHz, DMSO-d₆) δ 9.23 (s, 1H), 9.03 (s, 1H), 8.83 (s, 1H), 8.65 (d, J=24.8 Hz, 2H), 8.40 (s, 2H), 8.01 (s, 2H), 7.51 (s, 1H), 5.33 (t, J=5.7 Hz, 1H), 4.92-4.75 (m, 3H), 4.64 (d, J=5.7 Hz, 2H), 1.57 (d, J=6.7 Hz, 6H). ¹³C NMR (201 MHz, DMSO-d₆) δ 153.02, 150.24, 150.07, 148.75, 148.18, 140.67, 137.42, 134.31, 121.99, 120.87, 55.37, 47.35, 41.34, 22.68; HRMS (ESI+) m/z calcd for C₂₂H₂₂N₁₀O [M+H]⁺: 443.2056, found: 443.2052.

### Example 42: 2-(1-(6-(([2,3′-bipyridin]-5-ylmethyl)amino)-9-isopropyl-9H-purin-2-yl)-1H-1,2,3-triazol-4-yl)ethan-1-ol

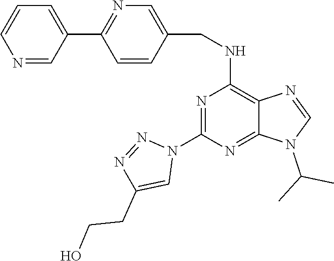 [SMILES: CC(C)n1cnc2c(NCc3ccc(-c4cccnc4)nc3)nc(-n3cc(CCO)nn3)nc21]

¹H NMR (400 MHz, CDCl₃) δ9.07 (d, J=1.7 Hz, 1H), 8.72 (d, J=1.8 Hz, 1H), 8.56 (dd, J=4.8, 1.5 Hz, 1H), 8.29 (s, 1H), 8.22 (d, J=8.0 Hz, 1H), 7.88-7.77 (m, 2H), 7.64 (d, J=8.2 Hz, 1H), 7.32 (dd, J=7.7, 4.9 Hz, 1H), 6.70 (s, 1H), 4.96-4.76 (m, 3H), 3.95 (t, J=5.8 Hz, 2H), 2.99 (t, J=5.8 Hz, 2H), 1.54 (d, J=6.8 Hz, 6H). ¹³C NMR (101 MHz, CDCl₃) δ 153.62, 149.62, 149.19, 147.82, 145.39, 138.49, 136.82, 134.33, 133.39, 123.63, 121.17, 120.28, 118.95, 61.29, 47.21, 29.01, 22.76; HRMS (ESI+) m/z calcd for C₂₃H₂₄N₁₀O [M+H]⁺: 457.2213, found: 457.2219.

The process for preparing the compound of Example 43 to the compound of Example 45 is as follows.

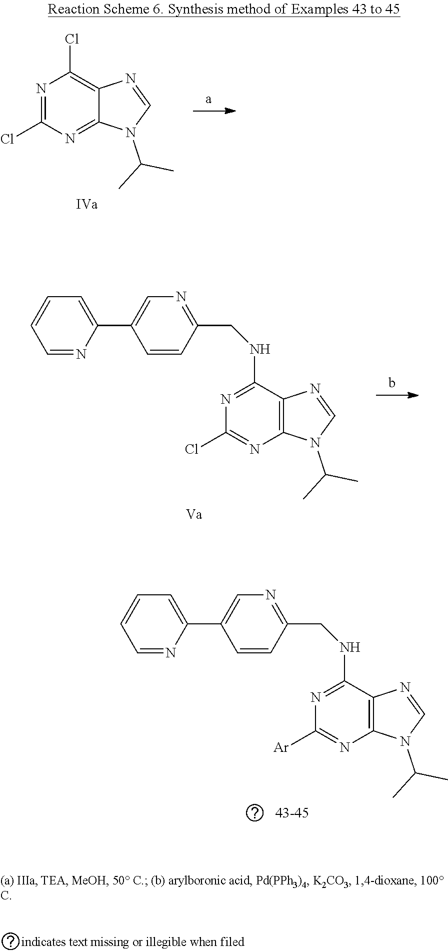

### Example 43: 3-(6-(([2,3′-bipyridin]-6′-ylmethyl)amino)-9-isopropyl-9H-purin-2-yl)phenol

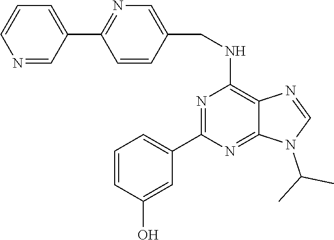 [SMILES: CC(C)n1cnc2c(NCc3ccc(-c4cccnc4)nc3)nc(-c3cccc(O)c3)nc21]

¹H NMR (400 MHz, CDCl₃) δ 9.07 (s, 1H), 8.68 (d, J=4.7 Hz, 1H), 8.13 (s, 1H), 7.94 (s, 1H), 7.78 (d, J=29.4 Hz, 3H), 7.68-7.57 (m, 1H), 7.48 (d, J=8.1 Hz, 1H), 7.27 (d, J=5.8 Hz, 1H), 7.24 (m, 1H), 6.92 (dd, J=8.0, 1.8 Hz, 1H), 6.89-6.72 (m, 1H), 5.04 (s, 2H), 4.85 (dt, J=13.5, 6.8 Hz 1H), 1.59 (d, J=6.4 Hz, 6H).

### Example 44: N-([2,3′-bipyridin]-6′-ylmethyl)-9-isopropyl-2-(5-methoxypyridin-3-yl)-9H-purin-6-amine

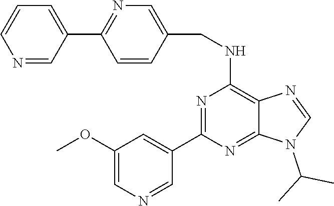 [SMILES: COc1cncc(-c2nc(NCc3ccc(-c4cccnc4)nc3)c3ncn(C(C)C)c3n2)c1]

¹H NMR (400 MHz, CDCl₃) δ 9.31 (d, J=1.6 Hz, 1H), 9.17 (d, J=1.8 Hz, 1H), 8.71 (d, J=4.2 Hz, 1H), 8.34 (d, J=2.9 Hz, 1H), 8.30 (dd, J=8.2, 2.3 Hz, 1H), 8.26 (d, J=1.6 Hz, 1H), 7.87 (s, 1H), 7.82-7.76 (m, 1H), 7.74 (d, J=7.9 Hz, 1H), 7.53 (d, J=8.2 Hz, 1H), 7.31-7.27 (m, 1H), 6.90 (s, 1H), 5.15 (s, 2H), 4.93 (dt, J=13.6, 6.8 Hz, 1H), 3.95 (s, 3H), 1.67 (d, J=6.8 Hz, 6H).

### Example 45: N-([2,3′-bipyridin]-6′-ylmethyl)-2-(6-aminopyridin-3-yl)-9-isopropyl-9H-purin-6-amine

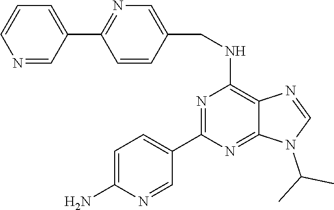 [SMILES: CC(C)n1cnc2c(NCc3ccc(-c4cccnc4)nc3)nc(-c3ccc(N)nc3)nc21]

¹H NMR (400 MHz, CDCl₃) δ 9.17 (dd, J=7.3, 1.9 Hz, 2H), 8.71 (d, J=4.3 Hz, 1H), 8.50 (dd, J=8.6, 2.2 Hz, 1H), 8.28 (dd, J=8.1, 2.3 Hz, 1H), 7.80 (s, 1H), 7.77 (dd, J=7.3, 1.7 Hz, 1H), 7.74 (d, J=7.9 Hz, 1H), 7.52 (d, J=8.1 Hz, 1H), 7.30-7.27 (m, 1H), 6.77 (s, 1H), 6.55 (d, J=8.7 Hz, 1H), 5.14 (s, 2H), 4.90 (dt, J=13.7, 6.8 Hz, 1H), 4.67 (s, 2H), 1.64 (d, J=6.8 Hz, 6H).

The process for preparing the compound of Example 46 to the compound of Example 48 is as follows.

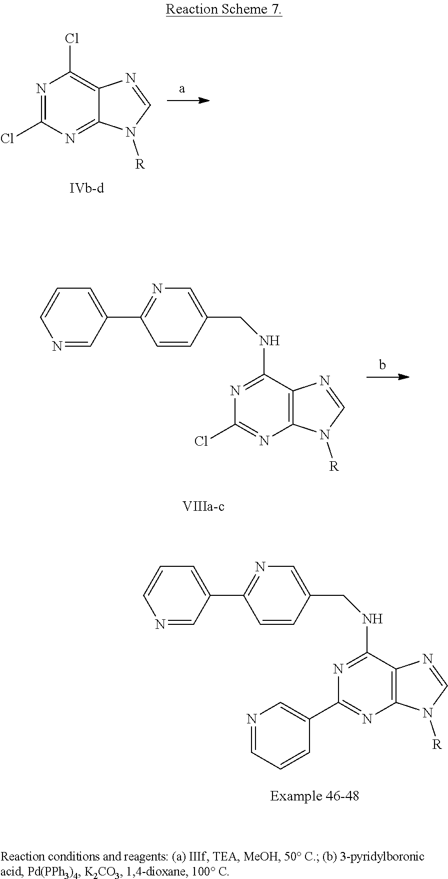

### Example 46: N-([2,3′-bipyridin]-5-ylmethyl)-9-ethyl-2-(pyridin-3-yl)-9H-purin-6-amine

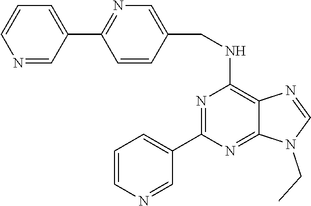 [SMILES: CCn1cnc2c(NCc3ccc(-c4cccnc4)nc3)nc(-c3cccnc3)nc21]

¹H NMR (400 MHz, CDCl₃) δ 9.71 (d, J=1.5 Hz, 1H), 9.20 (d, J=1.8 Hz, 1H), 8.88-8.76 (m, 2H), 8.67 (ddd, J=7.8, 4.8, 1.4 Hz, 2H), 8.33 (dt, J=8.0, 1.8 Hz, 1H), 7.91 (dd, J=8.1, 2.1 Hz, 1H), 7.85 (s, 1H), 7.74 (d, J=8.1 Hz, 1H), 7.44 (ddd, J=17.0, 7.9, 4.9 Hz, 2H), 6.49 (t, J=5.6 Hz, 1H), 5.08 (s, 2H), 4.35 (q, J=7.3 Hz, 2H), 1.62 (t, J=7.3 Hz, 3H). ¹³C NMR (101 MHz, CDCl₃) δ 156.89, 154.25, 153.81, 150.24, 149.83, 149.50, 148.12, 140.03, 136.42, 135.45, 134.50, 134.24, 134.18, 134.04, 123.58, 123.17, 120.30, 38.89, 15.52; HRMS (ESI+) m/z calcd for C₂₃H₂₀N₈ [M+H]⁺: 409.1889, found: 409.1882.

### Example 47: N-([2,3′-bipyridin]-5-ylmethyl)-9-cyclopentyl-2-(pyridin-3-yl)-9H-purin-6-amine

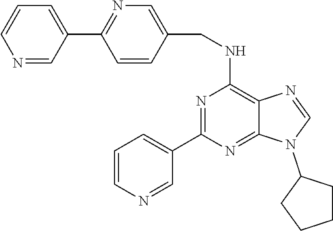 [SMILES: c1cncc(-c2ccc(CNc3nc(-c4cccnc4)nc4c3ncn4C3CCCC3)cn2)c1]

¹H NMR (400 MHz, CDCl₃) δ 9.72 (s, 1H), 9.21 (s, 1H), 8.92-8.79 (m, 2H), 8.68 (s, 2H), 8.36 (d, J=7.9 Hz, 1H), 7.98-7.85 (m, 2H), 7.76 (d, J=8.2 Hz, 1H), 7.59-7.40 (m, 2H), 6.45 (s, 1H), 5.17-4.90 (m, 3H), 2.38 (d, J=7.8 Hz, 2H), 2.08 (ddd, J=27.0, 13.6, 6.9 Hz, 4H), 1.89 (d, J=6.7 Hz, 2H). ¹³C NMR (101 MHz, CDCl₃) 5154.24, 153.90, 150.10, 149.86, 149.77, 149.55, 148.16, 139.20, 136.46, 135.53, 134.25, 134.03, 123.60, 123.21, 120.34, 56.30, 32.77, 24.17; HRMS (ESI+) m/z calcd for C₂₆H₂₄N₈ [M+H]⁺: 449.2202, found: 449.2213.

### Example 48: N-([2,3′-bipyridin]-5-ylmethyl)-2-(pyridin-3-yl)-9-(tetrahydro-2H-pyran-4-yl)-9H-purin-6-amine

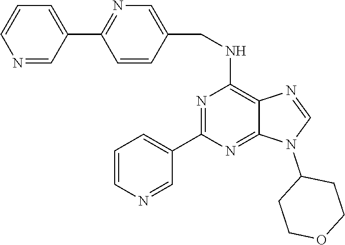 [SMILES: c1cncc(-c2ccc(CNc3nc(-c4cccnc4)nc4c3ncn4C3CCOCC3)cn2)c1]

¹H NMR (400 MHz, CDCl₃) δ 9.71 (d, J=1.6 Hz, 1H), 9.21 (d, J=1.7 Hz, 1H), 8.84 (d, J=7.6 Hz, 2H), 8.74-8.63 (m, 2H), 8.41-8.32 (m, 1H), 7.97-7.88 (m, 2H), 7.77 (d, J=8.1 Hz, 1H), 7.51 (dd, J=7.8, 4.9 Hz, 1H), 7.45 (dd, J=8.0, 4.4 Hz, 1H), 6.43 (s, 1H), 5.09 (s, 2H), 4.81 (ddd, J=16.1, 11.9, 4.3 Hz, 1H), 4.23 (dd, J=11.4, 4.0 Hz, 2H), 3.71 (dd, J=11.9, 10.0 Hz, 2H), 2.31 (dd, J=12.2, 4.2 Hz, 2H), 2.24-2.18 (m, 2H). ¹³C NMR (101 MHz, CDCl₃) δ 156.80, 153.82, 150.20, 149.74, 149.52, 148.03, 138.27, 136.46, 135.60, 134.55, 134.34, 134.16, 133.98, 123.64, 123.51, 123.26, 120.33, 67.07, 51.77, 33.03; HRMS (ESI+) m/z calcd for C₂₆H₂₄N₈O [M+H]⁺: 465.2151, found: 465.2149.

The process for preparing the compound of Example 49 to the compound of Example 54 is as follows.

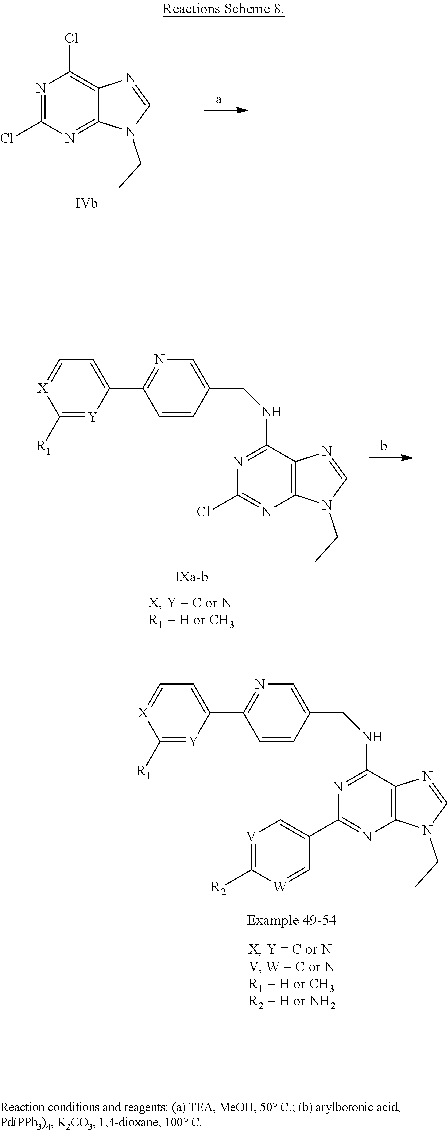

### Example 49: 2-(6-aminopyridin-3-yl)-9-ethyl-N-((2′-methyl-[2,4′-bipyridin]-5-yl)methyl)-9H-purin-6-amine

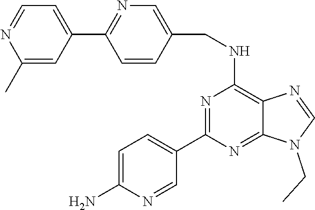 [SMILES: CCn1cnc2c(NCc3ccc(-c4ccnc(C)c4)nc3)nc(-c3ccc(N)nc3)nc21]

¹H NMR (400 MHz, CDCl₃) δ 9.20 (d, J=1.9 Hz, 1H), 8.83 (d, J=1.8 Hz, 1H), 8.61 (d, J=5.2 Hz, 1H), 8.50 (dd, J=8.6, 2.2 Hz, 1H), 7.91 (dd, J=8.1, 2.2 Hz, 1H), 7.76 (dd, J=12.1, 5.7 Hz, 3H), 7.68-7.62 (m, 1H), 6.59 (t, J=7.7 Hz, 1H), 6.26 (s, 1H), 5.06 (d, J=4.6 Hz, 2H), 4.70 (s, 2H), 4.32 (q, J=7.3 Hz, 2H), 2.66 (s, 3H), 1.60 (t, J=7.3 Hz, 3H). ¹³C NMR (101 MHz, CDCl₃+CD₃OD) 5159.17, 158.89, 157.77, 153.57, 149.41, 149.26, 148.46, 146.80, 139.19, 137.87, 136.56, 135.51, 124.91, 120.92, 120.87, 118.38, 118.05, 108.03, 38.79, 24.13, 15.44; HRMS (ESI+) m/z calcd for C₂₄H₂₃N₉ [M+H]⁺: 438.2155, found: 438.2152.

### Example 50: 2-(2-aminopyrimidin-5-yl)-9-ethyl-N-((2′-methyl-[2,4′-bipyridin]-5-yl)methyl)-9H-purin-6-amine

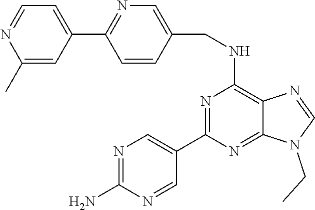 [SMILES: CCn1cnc2c(NCc3ccc(-c4ccnc(C)c4)nc3)nc(-c3cnc(N)nc3)nc21]

¹H NMR (400 MHz, DMSO-d₆) δ9.09 (s, 2H), 8.81 (s, 1H), 8.52 (t, J=7.2 Hz, 2H), 8.18 (s, 1H), 8.05 (d, J=8.1 Hz, 1H), 7.97 (dd, J=8.2, 1.9 Hz, 1H), 7.90 (s, 1H), 7.02 (s, 2H), 4.84 (s, 2H), 4.23 (q, J=7.2 Hz, 2H), 2.54 (s, 3H), 1.45 (t, J=7.2 Hz, 3H). ¹³C NMR (101 MHz, CDCl₃+CD₃OD) δ 166.78, 162.68, 162.39, 159.71, 157.94, 157.41, 153.20, 152.93, 150.90, 143.47, 140.57, 139.46, 126.30, 125.02, 124.99, 122.47, 122.13, 42.82, 27.76, 19.29. HRMS (ESI) calcd for C₂₃H₂₂N₁₀ [M+H]⁺: 439.2102, found: 439.2122; HRMS (ESI+) m/z calcd for C₂₄H₂₂N₁₀ [M+H]⁺: 439.2102, found: 439.2122.

### Example 51: 9-ethyl-N-((2′-methyl-[2,4′-bipyridin]-5-yl)methyl)-2-(pyrimidin-5-yl)-9H-purin-6-amine

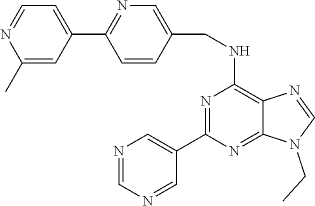 [SMILES: CCn1cnc2c(NCc3ccc(-c4ccnc(C)c4)nc3)nc(-c3cncnc3)nc21]

¹H NMR (400 MHz, CDCl₃) δ 9.71 (s, 2H), 9.28 (s, 1H), 8.83 (d, J=1.9 Hz, 1H), 8.61 (d, J=5.3 Hz, 1H), 7.91 (dd, J=8.1, 2.2 Hz, 1H), 7.86 (s, 1H), 7.78 (d, J=7.7 Hz, 2H), 7.66 (d, J=5.2 Hz, 1H), 6.31 (s, 1H), 5.08 (s, 2H), 4.36 (q, J=7.3 Hz, 2H), 2.66 (s, 3H), 1.63 (t, J=7.3 Hz, 3H). ¹³C NMR (101 MHz, CDCl₃) δ 159.16, 158.94, 156.47, 154.68, 154.35, 154.07, 149.71, 149.38, 146.25, 140.37, 136.33, 134.67, 131.73, 120.67, 120.51, 118.10, 39.01, 24.55, 15.51; HRMS (ESI+) m/z calcd for C₂₃H₂₁N₉ [M+H]⁺: 424.1998, found: 424.2007.

### Example 52: 5-(9-ethyl-6-(((2′-methyl-[2,4′-bipyridin]-5-yl)methyl)amino)-9H-purin-2-yl)-1,3-dihydro-2H-benzo[d]imidazol-2-one

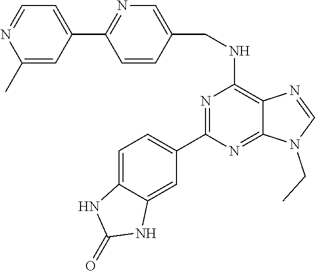 [SMILES: CCn1cnc2c(NCc3ccc(-c4ccnc(C)c4)nc3)nc(-c3ccc4[nH]c(=O)[nH]c4c3)nc21]

¹H NMR (400 MHz, DMSO-d₆) δ 10.77 (s, 1H), 10.69 (s, 1H), 8.83 (s, 1H), 8.52 (d, J=5.2 Hz, 1H), 8.43 (s, 1H), 8.18 (s, 1H), 8.13-7.97 (m, 4H), 7.89 (s, 1H), 7.80 (d, J=5.3 Hz, 1H), 7.00 (d, J=8.2 Hz, 1H), 4.89 (s, 2H), 4.25 (q, J=7.2 Hz, 2H), 2.54 (s, 3H), 1.47 (t, J=7.2 Hz, 3H).

### Example 53: N-([2,2′-bipyridin]-5-ylmethyl)-2-(6-aminopyridin-3-yl)-9-ethyl-9H-purin-6-amine

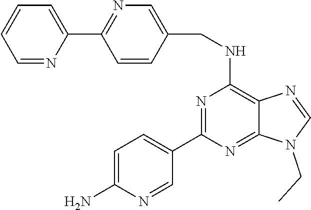 [SMILES: CCn1cnc2c(NCc3ccc(-c4ccccn4)nc3)nc(-c3ccc(N)nc3)nc21]

¹H NMR (400 MHz, CDCl₃) δ9.22 (d, J=1.8 Hz, 1H), 8.79 (d, J=1.7 Hz, 1H), 8.69 (d, J=3.9 Hz, 1H), 8.51 (dd, J=8.6, 2.2 Hz, 1H), 8.39 (dd, J=7.8, 6.3 Hz, 2H), 7.92 (dd, J=8.2, 2.2 Hz, 1H), 7.83 (td, J=7.8, 1.8 Hz, 1H), 7.77 (s, 1H), 7.35-7.30 (m, 1H), 6.58 (d, J=8.6 Hz, 1H), 6.11 (s, 1H), 5.08 (s, 2H), 4.64 (s, 2H), 4.32 (q, J=7.3 Hz, 2H), 1.61 (t, J=7.3 Hz, 3H). ¹³C NMR (101 MHz, DMSO-d₆) δ 161.02, 157.39, 155.70, 154.34, 149.60, 149.11, 148.96, 140.65, 137.57, 136.92, 136.74, 124.34, 123.08, 120.76, 120.55, 107.52, 38.42, 15.79; HRMS (ESI+) m/z calcd for C₂₃H₂₁N₉ [M+H]⁺: 424.1998, found: 424.2008.

### Example 54: N-([2,2′-bipyridin]-5-ylmethyl)-2-(2-aminopyrimidin-5-yl)-9-ethyl-9H-purin-6-amine

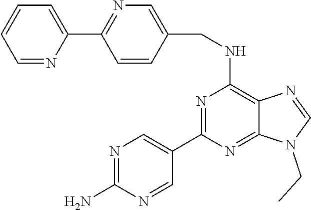 [SMILES: CCn1cnc2c(NCc3ccc(-c4ccccn4)nc3)nc(-c3cnc(N)nc3)nc21]

¹H NMR (400 MHz, DMSO-d₆) δ9.09 (s, 2H), 8.77 (s, 1H), 8.69-8.63 (m, 1H), 8.50 (s, 1H), 8.35 (t, J=7.8 Hz, 2H), 8.18 (s, 1H), 8.00-7.88 (m, 2H), 7.43 (ddd, J=7.5, 4.8, 1.1 Hz, 1H), 7.01 (s, 2H), 4.85 (s, 2H), 4.23 (q, J=7.2 Hz, 2H), 1.46 (t, J=7.3 Hz, 3H). ¹³C NMR (101 MHz, DMSO-d₆) 5164.33, 158.16, 155.65, 154.34, 149.67, 149.13, 141.02, 137.69, 136.85, 136.76, 124.45, 120.78, 120.57, 38.50, 15.76; HRMS (ESI+) m/z calcd for C₂₂H₂₀N₁₀ [M+H]⁺: 425.1951, found: 425.1672.

The process for preparing the compound of Example 55 to the compound of Example 60 is as follows.

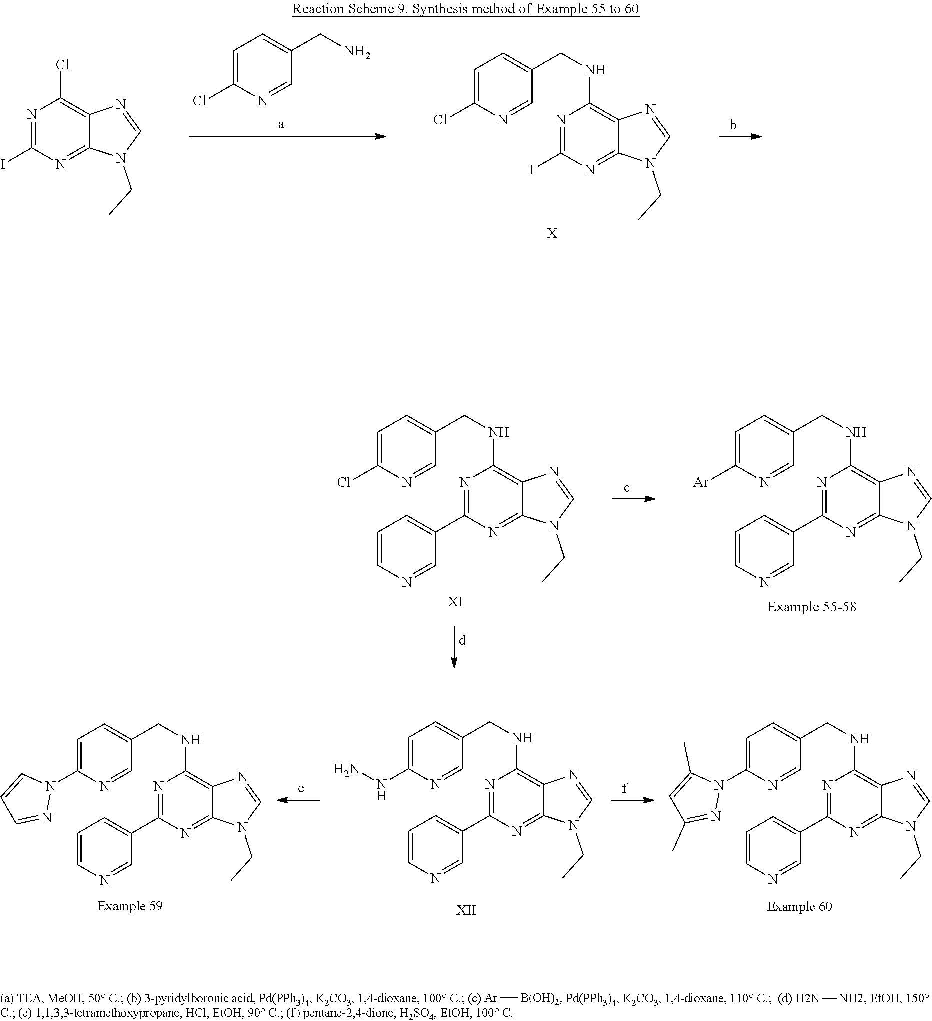

Synthesis of Intermediate (X) (N-((6-chloropyridin-3-yl)methyl)-9-ethyl-2-iodo-9H-purin-6-amine)

(6-chloropyridin-3-yl)methanamine (4 g, 1.2 eq) and triethylamine (3 mL, 3.0 eq) were added to a solution of 6-chloro-9-ethyl-2-iodo-9H-purine (4 g, 1.0 equivalents) dissolved in 10 mL of methanol at room temperature, and the mixture was stirred for 12 hours while being heated to 50° C. After methanol was evaporated, the residue was dissolved in ethyl acetate, and then washed with water, and the residue was removed with MgSO₄, and then a product was then separated by column chromatography using 2% methanol/dichloromethane.

Synthesis of Intermediate (XI) (N-((6-chloropyridin-3-yl)methyl)-9-ethyl-2-(pyridin-3-yl)-9H-purin-6-amine)

Intermediate X (4.2 g, 1.0 eq), tetrakis(triphenylphosphine)palladium (0.1 eq), and 3-pyridylboronic acid were added to 1,4-dioxane (6 mL), and 2 mL of a 2 M K₂CO₃ aqueous solution was added to the resulting mixture under nitrogen conditions with stirring. The vigorously stirred mixture was heated to 110° C. and stirred for 12 hours. After cooling, the mixture was filtered over a bed of Celite 545 and the organic layer was extracted with ethyl acetate. The solvent was evaporated under vacuum and the residue was purified by column chromatography using 4% MeOH/dichloromethane.

Synthesis of Intermediate (XII)(9-ethyl-N-((6-hydrazineylpyridin-3-yl)methyl)-2-(pyridin-3-yl)-9H-purin-6-amine)

Intermediate XI (1.0 g) and NH₂NH₂·H₂O (2 mL) were added to ethanol (1 mL) at room temperature, and the reaction mixture was heated to 150° C. with stirring overnight. After the reaction was terminated, the resulting product was cooled to room temperature, water (10 mL) was added thereto, and the solid Intermediate XII was filtered to obtain 850 mg (a yield of 85%). ¹H NMR (400 MHz, DMSO-d₆) δ 9.55 (s, 1H), 8.74-8.60 (m, 2H), 8.38 (s, 1H), 8.24 (d, J=15.1 Hz, 1H), 8.12 (s, 1H), 7.61-7.43 (m, 2H), 7.26 (s, 1H), 6.64 (d, J=8.6 Hz, 1H), 4.64 (s, 2H), 4.26 (q, J=7.3 Hz, 2H), 4.04 (s, 2H), 1.47 (t, J=7.3 Hz, 3H).

### Example 55: 9-ethyl-2-(pyridin-3-yl)-N-((6-(thiazol-2-yl)pyridin-3-yl)methyl)-9H-purin-6-amine

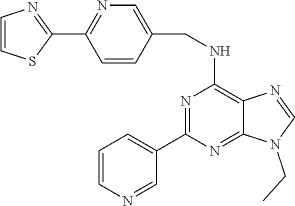 [SMILES: CCn1cnc2c(NCc3ccc(-c4nccs4)nc3)nc(-c3cccnc3)nc21]

¹H NMR (400 MHz, CDCl₃) δ 9.70 (s, 1H), 8.84 (s, 1H), 8.76 (d, J=8.0 Hz, 1H), 8.73-8.67 (m, 2H), 8.33 (s, 1H), 7.86 (dd, J=8.9, 2.8 Hz, 2H), 7.69 (d, J=8.1 Hz, 1H), 7.44 (dd, J=7.6, 4.7 Hz, 1H), 6.39 (s, 1H), 5.05 (s, 2H), 4.36 (q, J=7.3 Hz, 2H), 1.63 (t, J=7.3 Hz, 3H).

### Example 56: N-((6-(1H-pyrazol-4-yl)pyridin-3-yl)methyl)-9-ethyl-2-(pyridin-3-yl)-9H-purin-6-amine

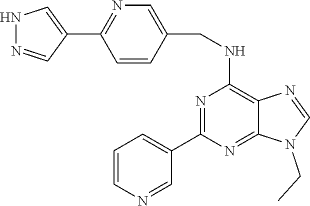 [SMILES: CCn1cnc2c(NCc3ccc(-c4cn[nH]c4)nc3)nc(-c3cccnc3)nc21]

¹H NMR (400 MHz, DMSO-d₆) δ13.01 (s, 1H), 9.54 (s, 1H), 8.73-8.50 (m, 4H), 8.26 (s, 2H), 8.00 (s, 1H), 7.82 (d, J=6.3 Hz, 1H), 7.63 (d, J=8.5 Hz, 1H), 7.52 (dd, J=7.9, 4.8 Hz, 1H), 4.80 (s, 2H), 4.28 (q, J=7.3 Hz, 2H), 1.48 (t, J=7.3 Hz, 3H).

### Example 57: N-((6-(1H-indazol-6-yl)pyridin-3-yl)methyl)-9-ethyl-2-(pyridin-3-yl)-9H-purin-6-amine

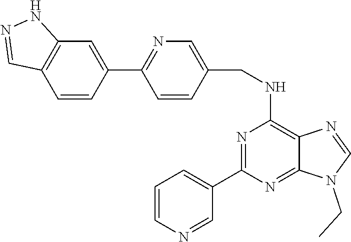 [SMILES: CCn1cnc2c(NCc3ccc(-c4ccc5cn[nH]c5c4)nc3)nc(-c3cccnc3)nc21]

¹H NMR (400 MHz, DMSO-d₆) δ13.17 (s, 1H), 9.53 (d, J=1.5 Hz, 1H), 8.80 (s, 1H), 8.72-8.57 (m, 3H), 8.27 (s, 1H), 8.18 (s, 1H), 8.10 (d, J=8.2 Hz, 1H), 7.97 (dd, J=21.5, 8.1 Hz, 2H), 7.82 (s, 2H), 7.52 (dd, J=7.9, 4.8 Hz, 1H), 4.88 (s, 2H), 4.28 (q, J=7.2 Hz, 2H), 1.48 (t, J=7.3 Hz, 3H).

### Example 58: 9-ethyl-N-((6-(furan-3-yl)pyridin-3-yl)methyl)-2-(pyridin-3-yl)-9H-purin-6-amine

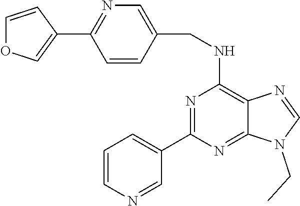 [SMILES: CCn1cnc2c(NCc3ccc(-c4ccoc4)nc3)nc(-c3cccnc3)nc21]

¹H NMR (400 MHz, CDCl₃) δ9.61 (d, J=1.4 Hz, 1H), 8.67 (dt, J=8.0, 1.9 Hz, 1H), 8.63-8.56 (m, 2H), 7.94 (dd, J=1.4, 0.8 Hz, 1H), 7.73 (s, 1H), 7.71 (dd, J=8.1, 2.3 Hz, 1H), 7.41 (t, J=1.7 Hz, 1H), 7.37-7.32 (m, 2H), 6.80 (dd, J=1.8, 0.8 Hz, 1H), 6.27 (s, 1H), 4.92 (s, 2H), 4.25 (q, J=7.3 Hz, 2H), 1.52 (t, J=7.3 Hz, 3H).

### Example 59: N-((6-(1H-pyrazol-1-yl)pyridin-3-yl)methyl)-9-ethyl-2-(pyridin-3-yl)-9H-purin-6-amine

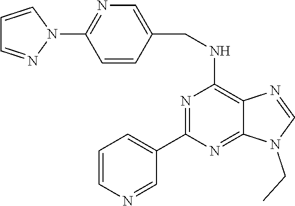 [SMILES: CCn1cnc2c(NCc3ccc(-n4cccn4)nc3)nc(-c3cccnc3)nc21]

¹H NMR (400 MHz, DMSO-d₆) δ9.53 (d, J=1.4 Hz, 1H), 8.70-8.56 (m, 5H), 8.27 (s, 1H), 8.07-8.02 (m, 1H), 7.89 (d, J=8.3 Hz, 1H), 7.79 (d, J=1.0 Hz, 1H), 7.52 (dd, J=7.2, 4.8 Hz, 1H), 6.57-6.53 (m, 1H), 4.87 (s, 2H), 4.28 (q, J=7.2 Hz, 2H), 1.49 (t, J=7.3 Hz, 3H).

### Example 60: N-((6-(3,5-dimethyl-1H-pyrazol-1-yl)pyridin-3-yl)methyl)-9-ethyl-2-(pyridin-3-yl)-9H-purin-6-amine

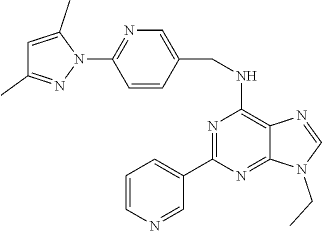 [SMILES: CCn1cnc2c(NCc3ccc(-n4nc(C)cc4C)nc3)nc(-c3cccnc3)nc21]

Intermediate XII (70 mg, 1 eq), pentane-2,4-dione (1.5 eq) and H₂SO₄ (1 eq) were added to ethanol (2 mL) solvent at room temperature, and the reaction mixture was refluxed with stirring. After the reaction was completed, the ethanol solvent was evaporated and the product was purified by column chromatography using 4% MeOH/dichloromethane. ¹H NMR (400 MHz, CDCl₃) δ 9.70 (d, J=1.5 Hz, 1H), 8.72 (dt, J=8.0, 1.9 Hz, 1H), 8.68 (dd, J=4.8, 1.7 Hz, 1H), 8.52 (d, J=2.0 Hz, 1H), 7.88 (dd, J=8.5, 2.3 Hz, 1H), 7.82 (t, J=4.0 Hz, 2H), 7.40 (ddd, J=8.0, 4.8, 0.7 Hz, 1H), 6.21 (s, 1H), 5.99 (s, 1H), 5.03 (s, 2H), 4.35 (q, J=7.3 Hz, 2H), 2.63 (s, 3H), 2.30 (s, 3H), 1.62 (t, J=7.3 Hz, 3H).

The process for preparing the compound of Example 61 to the compound of Example 62 is as follows.

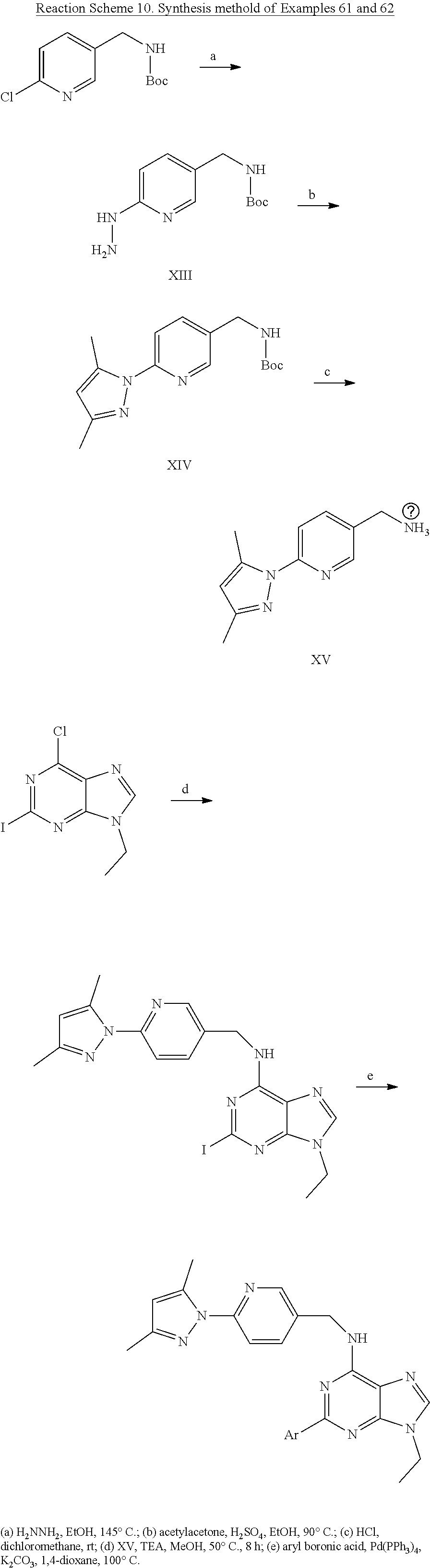

Synthesis Method of Intermediate (XV) 6-(3,5-dimethyl-1H-pyrazol-1-yl)pyridin-3-yl)methanaminium chloride

tert-Butyl(6-chloropyridin-3-yl)carbamate and NH₂NH₂·H₂O (2 mL) were added to ethanol (1 mL) and the reaction mixture was heated to 150° C. overnight. After the reaction was cooled to room temperature, water (10 mL) was added thereto and a solid desired product 2 was filtered to obtain 808 mg (a yield of 81%) of Intermediate XIII. The intermediate, acetylacetone (1.5 eq), and sulfuric acid (1 eq) were mixed with ethanol (2 mL) at room temperature, and the reaction mixture was refluxed with stirring. After the reaction, an ethanol solvent was evaporated and the residue was purified by column chromatography using 2% MeOH/dichloromethane to obtain Intermediate XIV at a yield of 71%. Thereafter, Intermediate XIV was dissolved in dichloromethane, and 2 mL of 4 N HCl (a 1,4-dixoane solution) was slowly added. The resulting mixture was continuously stirred at room temperature for 4 hours and a solid compound XV was filtered (a yield of 98%).

### Example 61: 2-(6-aminopyridin-3-yl)-N-((6-(3,5-dimethyl-1H-pyrazol-1-yl)pyridin-3-yl)methyl)-9-ethyl-9H-purin-6-amine

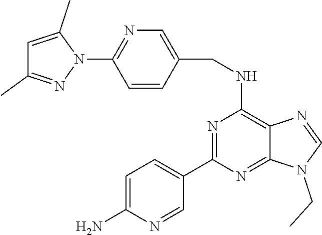 [SMILES: CCn1cnc2c(NCc3ccc(-n4nc(C)cc4C)nc3)nc(-c3ccc(N)nc3)nc21]

¹H NMR (400 MHz, DMSO-d₆) δ8.91 (d, J=2.1 Hz, 1H), 8.50 (s, 1H), 8.33 (s, 1H), 8.27 (dd, J=8.7, 2.2 Hz, 1H), 8.13 (s, 1H), 7.94 (d, J=2.3 Hz, 1H), 7.73 (d, J=8.5 Hz, 1H), 6.48 (d, J=8.7 Hz, 1H), 6.27 (s, 2H), 6.07 (s, 1H), 4.80 (s, 2H), 4.21 (q, J=7.2 Hz, 2H), 2.52 (d, J=3.5 Hz, 3H), 2.16 (s, 3H), 1.44 (t, J=7.3 Hz, 3H).

### Example 62: 2-(2-aminopyrimidin-5-yl)-N-((6-(3,5-dimethyl-1H-pyrazol-1-yl)pyridin-3-yl)methyl)-9-ethyl-9H-purin-6-amine

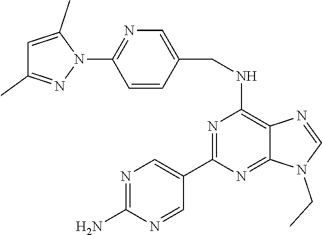 [SMILES: CCn1cnc2c(NCc3ccc(-n4nc(C)cc4C)nc3)nc(-c3cnc(N)nc3)nc21]

¹H NMR (400 MHz, DMSO-d₆) δ9.09 (s, 2H), 8.51 (s, 2H), 8.18 (s, 1H), 7.96 (dd, J=8.5, 2.2 Hz, 1H), 7.74 (d, J=8.4 Hz, 1H), 7.02 (s, 2H), 6.08 (s, 1H), 4.81 (s, 2H), 4.23 (q, J=7.3 Hz, 2H), 2.53 (s, 3H), 2.18 (s, 3H), 1.46 (t, J=7.3 Hz, 3H).

The process for preparing the compound of Example 63 to the compound of Example 66 is as follows.

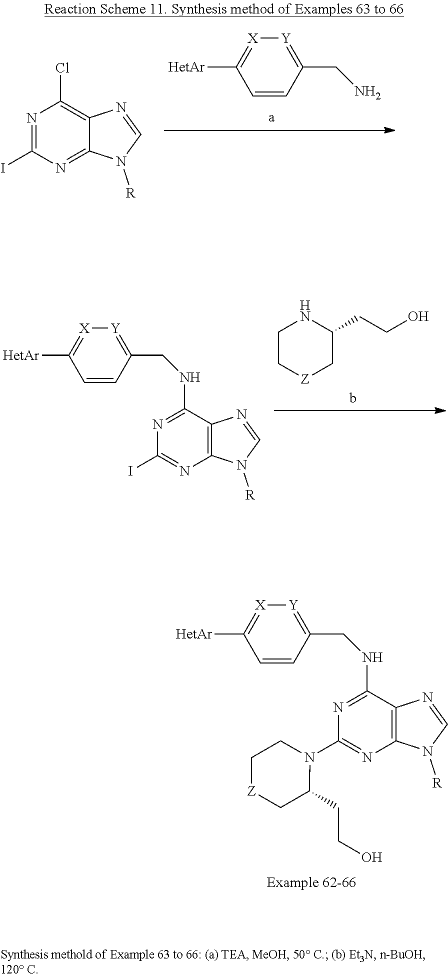

### Example 63: (R)-2-(4-(6-(([2,3′-bipyridin]-5-ylmethyl)amino)-9-isopropyl-9H-purin-2-yl)morpholin-3-yl)ethan-1-ol

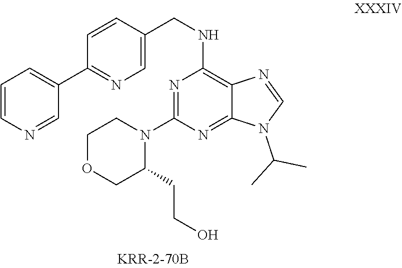 [SMILES: CC(C)n1cnc2c(NCc3ccc(-c4cccnc4)nc3)nc(N3CCOC[C@H]3CCO)nc21]

¹H NMR (400 MHz, CDCl₃) δ 9.21 (s, 1H), 8.78 (s, 1H), 8.68 (d, J=4.8 Hz, 1H), 8.34 (d, J=7.9 Hz, 1H), 7.87 (d, J=8.4 Hz, 1H), 7.76 (d, J=8.1 Hz, 1H), 7.68 (s, 1H), 7.44 (dd, J=7.9, 4.8 Hz, 1H), 6.78-6.57 (m, 1H), 4.87 (s, 2H), 4.68 (dd, J=24.2, 12.4 Hz, 3H), 3.96 (d, J=11.3 Hz, 1H), 3.76 (dt, J=42.3, 19.6 Hz, 3H), 3.59-3.49 (m, 1H), 3.49-3.39 (m, 1H), 3.24-3.11 (m, 1H), 2.43-2.29 (m, 1H), 1.88 (s, 2H), 1.65-1.45 (m, 6H).

### Example 64: (S)-2-(1-(9-isopropyl-6-(((2′-methyl-[2,4′-bipyridin]-5-yl)methyl)amino)-9H-purin-2-yl)piperidin-2-yl)ethan-1-ol

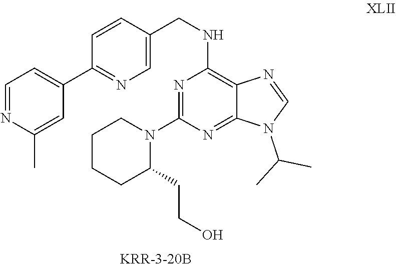 [SMILES: Cc1cc(-c2ccc(CNc3nc(N4CCCC[C@H]4CCO)nc4c3ncn4C(C)C)cn2)ccn1]

¹H NMR (400 MHz, CDCl₃) δ 8.78 (d, J=1.7 Hz, 1H), 8.62 (d, J=5.2 Hz, 1H), 7.86 (dd, J=8.2, 2.2 Hz, 1H), 7.81-7.75 (m, 2H), 7.70-7.65 (m, 2H), 6.15 (s, 1H), 4.97 (s, 2H), 4.79 (dt, J=13.1, 6.5 Hz, 1H), 4.37 (dd, J=8.2, 4.6 Hz, 2H), 3.96-3.73 (m, 2H), 3.46 (dd, J=15.3, 8.9 Hz, 1H), 3.13-3.03 (m, 1H), 2.86 (ddd, J=50.8, 28.8, 20.4 Hz, 2H), 2.66 (d, J=7.0 Hz, 3H), 2.36 (s, 1H), 1.87 (ddd, J=15.5, 11.7, 7.0 Hz, 3H), 1.59 (t, J=6.6 Hz, 6H).

### Example 65: (S)-2-(1-(6-(((6-(3,5-dimethyl-TH-pyrazol-1-yl)pyridin-3-yl)methyl)amino)-9-ethyl-9H-purin-2-yl)piperidin-2-yl)ethan-1-ol

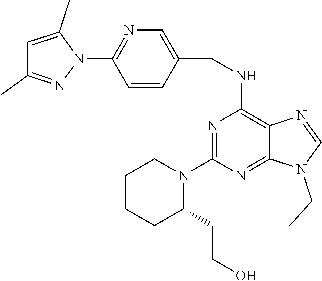 [SMILES: CCn1cnc2c(NCc3ccc(-n4nc(C)cc4C)nc3)nc(N3CCCC[C@H]3CCO)nc21]

¹H NMR (400 MHz, CDCl₃) δ 8.41 (d, J=1.6 Hz, 1H), 7.79 (dd, J=8.5, 2.2 Hz, 1H), 7.77-7.73 (m, 1H), 7.41 (s, 1H), 6.42 (s, 1H), 5.96 (s, 1H), 5.00-4.92 (m, 1H), 4.89-4.80 (m, 1H), 4.77 (s, 2H), 4.02 (q, J=7.3 Hz, 2H), 3.57 (dd, J=11.8, 1.9 Hz, 1H), 3.36 (td, J=11.7, 2.3 Hz, 1H), 2.81-2.71 (m, 1H), 2.59 (s, 3H), 2.27 (s, 3H), 2.10 (dd, J=14.1, 12.0 Hz, 1H), 1.83-1.71 (m, 1H), 1.63 (dd, J=13.1, 2.6 Hz, 4H), 1.58 (d, J=4.9 Hz, 1H), 1.45 (t, J=7.2 Hz, 3H), 1.40 (d, J=10.7 Hz, 1H).

### Example 66: (R)-2-(4-(6-(((6-(3,5-dimethyl-1H-pyrazol-1-yl)pyridin-3-yl)methyl)amino)-9-ethyl-9H-purin-2-yl)morpholin-3-yl)ethan-1-ol

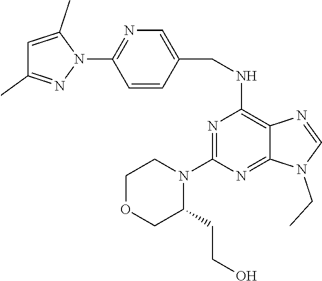 [SMILES: CCn1cnc2c(NCc3ccc(-n4nc(C)cc4C)nc3)nc(N3CCOC[C@H]3CCO)nc21]

¹H NMR (400 MHz, CDCl₃) δ 8.39 (s, 1H), 7.77 (d, J=7.2 Hz, 2H), 7.44 (s, 1H), 6.49 (s, 1H), 5.96 (d, J=7.9 Hz, 1H), 4.76 (s, 2H), 4.64 (t, J=12.7 Hz, 2H), 4.03 (q, J=7.3 Hz, 2H), 3.91 (dd, J=11.2, 3.1 Hz, 1H), 3.76 (d, J=11.3 Hz, 1H), 3.72 (dd, J=11.4, 3.0 Hz, 1H), 3.61 (d, J=11.3 Hz, 1H), 3.51 (td, J=12.0, 2.9 Hz, 1H), 3.36 (td, J=11.7, 2.2 Hz, 1H), 3.12 (td, J=13.5, 3.6 Hz, 1H), 2.59 (s, 3H), 2.37-2.29 (m, 1H), 2.27 (s, 3H), 1.78 (ddt, J=15.5, 11.5, 4.3 Hz, 1H), 1.45 (t, J=7.3 Hz, 3H).

### [Experimental Examples]

### Experimental Example 1. Measurement of Breast Cancer Growth Inhibitory Activity of Compounds

Breast cancer cells in a culture solution (100 μL volume/well, 2,500 SK-Br3 cells/well, 1,000 HCC-1954 cells/well) were seeded into a tissue culture-treated 96-well plate and cultured in a cell incubator for 24 hours. Serially diluted compounds (3-fold, 10 points at 10 mM, duplicated) were prepared in a compound plate, and 500 nL of the compound solution was transferred to the cells in an assay plate with pins using a pin tool system (JANUS liquid handler, PerkinElmer, USA), and then cultured in a cell incubator for 72 hours. Cell-titer Glo™ reagent (50 μL of a 5-fold diluted solution, Promega) was added to each well, and luminescence signals were measured using an Envision™ plate reader (PerkinElmer, USA). Titration curve fitting and IC₅₀ values were generated using Prism 7.0 s/w (GraphPad, San Diego, USA).

The SK-Br3 cell line was treated with the compounds of the present invention at various concentrations for 72 hours, and then the proliferation inhibitory activity was measured to calculate GI50 values. The in vitro IC₅₀ values for CDK12/cyclinK and GI₅₀ values for inhibiting the growth of the SK-Br3 cell line are each shown in Table 1.

TABLE 1

| | CDK12/CyclinK | SK-Br3 | HCC1954 |
|---|---|---|---|
| Example | (IC₅₀/μM) | (GI₅₀/μM) | (GI₅₀/μM) |
| 1 | 0.433 | 0.151 | 0.105 |
| 2 | 0.582 | 0.866 | 0.659 |
| 3 | 1.109 | 23.140 | 13.550 |
| 4 | 0.627 | 0.740 | 0.888 |
| 5 | 0.481 | 0.277 | 0.230 |
| 6 | 0.221 | 0.416 | 0.248 |
| 7 | 0.153 | 0.801 | 0.500 |
| 8 | 0.623 | 1.680 | 1.124 |
| 9 | 0.504 | 1.601 | 1.121 |
| 10 | 0.484 | 3.700 | 3.139 |
| 11 | 0.155 | 1.495 | 1.357 |
| 12 | nd | nd | nd |
| 13 | 0.179 | 0.597 | 0.400 |
| 14 | 0.104 | 2.598 | 1.875 |
| 15 | nd | nd | nd |
| 16 | 0.486 | 3.709 | 4.229 |
| 17 | 0.090 | 1.250 | 0.937 |
| 18 | 0.065 | 0.375 | 0.279 |
| 19 | 0.094 | 0.710 | 0.362 |
| 20 | 0.051 | 0.314 | 0.308 |
| 21 | 0.087 | 0.242 | 0.162 |
| 22 | 0.501 | 1.722 | 1.132 |
| 23 | 1.433 | 1.992 | 1.172 |
| 24 | nd | nd | nd |
| 25 | 0.619 | 2.577 | 2.622 |
| 26 | 0.170 | 0.615 | 0.340 |
| 27 | 0.150 | 0.813 | 0.497 |
| 28 | 0.147 | 1.747 | 1.308 |
| 29 | 0.164 | 1.718 | 1.274 |
| 30 | nd | nd | nd |
| 31 | 0.799 | 2.307 | 1.556 |
| 32 | 3.012 | 11.350 | 6.094 |
| 33 | 0.257 | 0.724 | 0.502 |
| 34 | 0.499 | 1.091 | 0.906 |
| 35 | 0.053 | 0.261 | 0.173 |
| 36 | 0.304 | 1.182 | 0.977 |
| 37 | 0.086 | 0.470 | 0.433 |
| 38 | 0.113 | 0.479 | 0.387 |
| 39 | nd | nd | nd |
| 40 | 2.345 | 5.176 | 3.454 |
| 41 | 2.051 | 13.270 | 9.996 |
| 42 | 0.539 | 8.293 | 6.499 |
| 43 | 0.713 | nd | nd |
| 44 | 1.530 | nd | nd |
| 45 | 0.221 | 0.169 | nd |
| 46 | 0.016 | 0.217 | 0.142 |
| 47 | 0.293 | 2.603 | 2.136 |
| 48 | >10 | >100 | 44.180 |
| 49 | 0.012 | 0.133 | 0.104 |
| 50 | 0.019 | 0.090 | 0.080 |
| 51 | 0.056 | 0.192 | 0.156 |
| 52 | nd | nd | 0.635 |
| 53 | 0.030 | 0.038 | 0.036 |
| 54 | 0.077 | 0.052 | 0.034 |
| 55 | nd | nd | 0.529 |
| 56 | nd | nd | 3.197 |
| 57 | nd | nd | 5.040 |
| 58 | nd | 0.031 | 0.029 |
| 59 | nd | 0.032 | 0.039 |
| 60 | nd | 0.102 | 0.097 |
| 61 | nd | 0.144 | 0.150 |
| 62 | nd | 0.066 | 0.085 |
| 63 | nd | nd | 0.168 |
| 64 | nd | nd | nd |
| 65 | nd | 0.035 | 0.038 |
| 66 | nd | 0.019 | 0.020 |
| THZ531 | 0.050 | 0.030 | 0.214 |
| Dinaciclib | <0.005 | 0.012 | 0.012 |

### Experimental Example 2. CyclinK Degradation Measurement, Inhibition of PolII CTD Ser2 Phosphorylation, and Suppression of Downstream Gene Expression

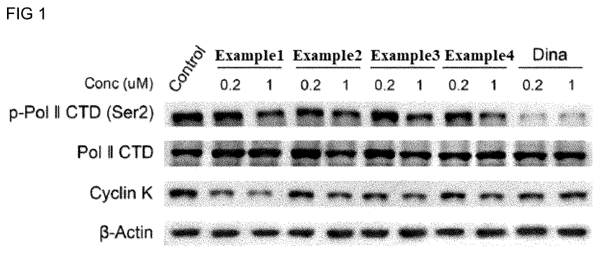

In order to confirm the intracellular CDK12 inhibition and cyclin K degradation of the compounds of the present invention compared to a dinaciclib control drug, an SK-Br3 cell line was treated with structurally identified representative example compounds at a concentration of 0.2 and 1 μM for 2 hours, and then western blot experiments were performed using a PolII CTD p-Ser2 antibody and a cyclinK antibody. As shown in FIG. 1, all tested compounds showed stronger cyclinK degradation ability compared to dinaciclib.

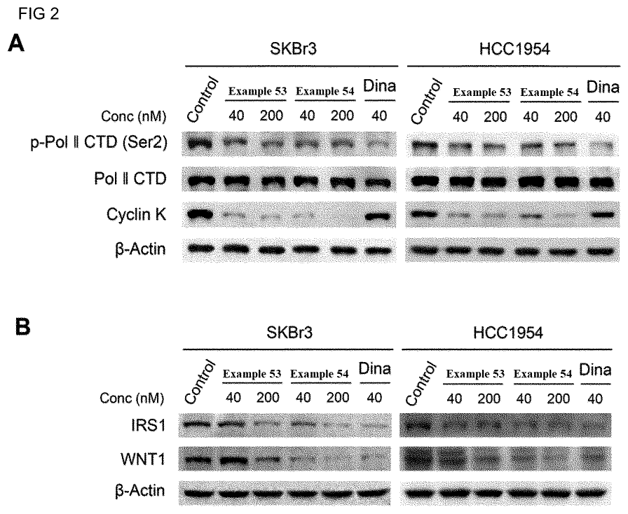

Further, intracellular target inhibition was investigated in SK-Br3 and HCC1954 cells after treatment with Compounds 53 and 54 at a concentration of 40 and 200 nM for 2 hours (FIG. 2). In both cells, cyclinK levels were greatly suppressed in a dose-dependent manner, indicating that both compounds act as a potent cyclinK degrader. In addition, both compounds showed a strong dose-dependent suppression of Pol II phosphorylation (p-CTD Ser2). As a result of investigating the same doses of the compounds after treatment for 24 hours, the expression of CDK12 downstream genes (IRS1 and WNT1) was strongly suppressed.

### Experimental Example 3. Synergistic Effect with Trastuzumab

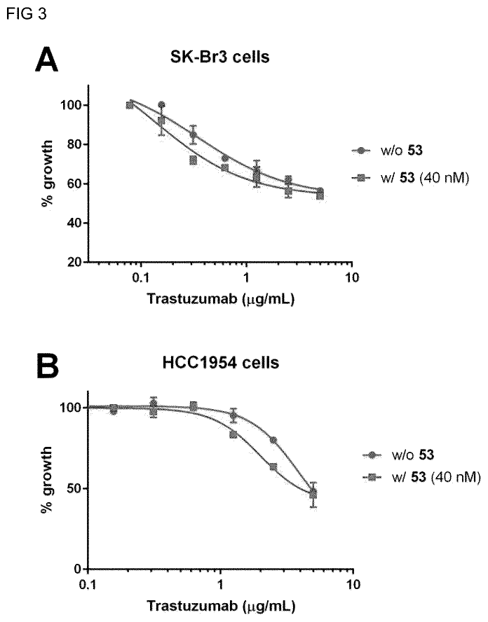

The effect of combined administration was investigated in SK-Br3 and HCC1954 cells. Cells were treated with various doses of trastuzumab for 72 hours in the absence or presence of the treatment of a single dose (40 nM), which has a GI₅₀ value similar to the GI₅₀ value of Compound 53 (FIG. 3). When trastuzumab was administered in combination with Compound 53 in both cell lines, the inhibitory activity of trastuzumab was slightly increased, indicating synergy between Compound 53 and trastuzumab in inhibiting the growth of HER2+ breast cancer cells regardless of their sensitivity to trastuzumab.

### Experimental Example 4. Prediction of Binding Mode for CDK12-DDB1 Complex

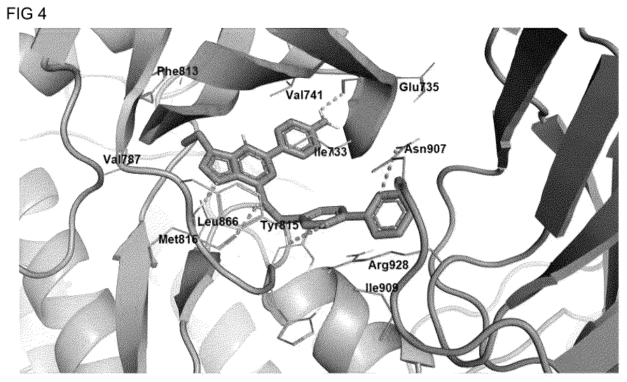

Docking analysis of Compound 53 was performed using a CDK12-DDB1 complex crystal structure (pdb id: 6td3) (FIG. 4). A hydrogen bond was predicted between the N7 and NH pair of a purine ring and the hinge Met816 backbone. In addition, the ethyl group at position 9 is present in a small hydrophobic pocket generated by the three hydrophobic side chains of Val787, Phe813, and Leu866, and the aminopyridine group at position 2 is predicted to form a hydrogen bond with the carbonyl backbone of Glu735 as well as to have hydrophobic interactions with Ile733 and Val741. The internal pyridine at position 6 was predicted to form a hydrogen bond with the side chain of Tyr815 and form hydrophobic contact with Ile733. Furthermore, the terminal pyridine at position 6 interacts with DDB1 through a hydrogen bond with Asn907 and hydrophobic interactions with the hydrophobic side chains of Ile909 and Arg928.

### Experimental Example 5. Kinome-Wide Inhibitory Activity Profiling

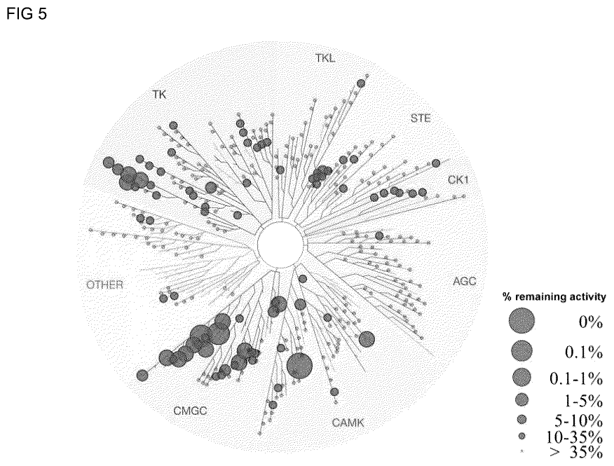

The activity of Compound 53 against a panel of human kinases was measured at a concentration of 10 μM. (FIG. 5). Among 371 human wild-type kinases, the following kinases were inhibited by 10 μM of Compound 53 by 90% or more—CDK1/cyclinA, CDK1/cyclinB, CDK1/cyclinE, CDK2/cyclinA, CDK2/cyclinA1, CDK2/cyclinO, CDK2/cyclinE, CDK2/cyclinE2, CDK3/cyclinE, CDK3/cyclinE2, CDK5/p25, CDK5/p35, CDK7/cyclinH, CDK9/cyclinK, CDK9/cyclinT1, CDK9/cyclinT2, CDK17/cyclinY, CDK18/cyclinY, LKB1, EPHA3/4/5/6, EPHB2, DYRK1A/B, MAK, MYLK4, GSK3b, FES, CHK2, PAK5, ERK1 (Table 2). These results indicate that Compound 53 is a pan-CDK inhibitor capable of strongly inhibiting not only CDK12/cyclinK but also other CDKs. Unlike THZ531, which is a selective CDK12 inhibitor which shows selective activity against SK-Br3 (GI50=30 nM) compared to HCC1954 cells (GI₅₀=214 nM), the comparable growth inhibitory activity of Compound 53 (SK-Br3 GI₅₀=38 nM, HCC1954 GI₅₀=36 nM) and dinaciclib (SK-Br3 GI₅₀=12 nM, HCC1954 GI₅₀=12 nM) against the two breast cancer cell lines is likely due to their ability to simultaneously inhibit multiple CDK kinases. Therefore, targeting multiple CDKs together with CDK12/cyclinK may be advantageous in overcoming trastuzumab resistance. In addition, these kinome-wide activity inhibition profiling results suggest that the CDK inhibitors of the present invention may be extended to inhibitors of other important kinases including EPH-family tyrosine kinases.

TABLE 2
Kinome-wide inhibitory ability (% residual activity)
profiling of Example 53 (concentration of 10 μM).
Mean and standard deviations for duplicate measurements

| | | % Residual | Standard |
|---|---|---|---|
| No | Kinase | Activity (mean) | deviation |
| 1 | LKB1 | 0 | 0.03 |
| 2 | CDK2/cyclin A1 | 0.16 | 0.12 |
| 3 | CDK2/cyclin O | 0.54 | 0.17 |
| 4 | CDK5/p35 | 0.67 | 0 |
| 5 | CDK9/cyclin T1 | 0.78 | 0.02 |
| 6 | CDK3/cyclin E | 0.99 | 0.02 |
| 7 | CDK9/cyclin K | 1.07 | 0.2 |
| 8 | CDK2/cyclin A | 1.19 | 0.14 |
| 9 | CDK5/P25 | 1.6 | 0.12 |
| 10 | CDK9/cyclin T2 | 1.95 | 0.06 |
| 11 | CDK1/cyclin B | 2.06 | 0.2 |
| 12 | CDK18/cyclin Y | 2.46 | 0.06 |
| | (PCTK3) | | |
| 13 | EPHB2 | 2.52 | 0.54 |
| 14 | CDK3/cyclin E2 | 2.63 | 0.3 |
| 15 | EPHA6 | 3.04 | 0.02 |
| 16 | DYRK1B | 3.19 | 0.05 |
| 17 | MAK | 3.3 | 0.04 |
| 18 | EPHA4 | 3.61 | 0.16 |
| 19 | CDK7/cyclin H | 3.81 | 0.26 |
| 20 | DYRK1/DYRKIA | 3.85 | 0.36 |
| 21 | CDK2/CYCLIN E | 4.01 | 0.01 |
| 22 | MYLK4 | 4.07 | 0.25 |
| 23 | CDK1/cyclin A | 4.65 | 0.03 |
| 24 | GSK3b | 5.76 | 0.03 |
| 25 | EPHA5 | 5.86 | 0.05 |
| 26 | FES/FPS | 6.37 | 0.02 |
| 27 | CDK17/cyclin Y | 7.25 | 0.66 |
| | (PCTK2) | | |
| 28 | CHK2 | 7.39 | 0.16 |
| 29 | CDK1/cyclin E | 7.49 | 0.88 |
| 30 | PAK5 | 7.55 | 0.13 |
| 31 | EPHA3 | 7.8 | 0.09 |
| 32 | ERK1 | 9.4 | 0.43 |
| 33 | CDK2/cyclin E2 | 9.78 | 0.85 |
| 34 | FLT4/VEGFR3 | 10.02 | 0.12 |
| 35 | MUSK | 10.19 | 1.36 |
| 36 | CK1d | 10.25 | 0.88 |
| 37 | GSK3a | 10.57 | 1.02 |
| 38 | CAMKK2 | 10.59 | 0.26 |
| 39 | ERK2/MAPK1 | 10.6 | 0.12 |
| 40 | CLK2 | 11.71 | 0.37 |
| 41 | CDK6/cyclin D3 | 12.86 | 0.41 |
| 42 | PAK4 | 12.92 | 0.21 |
| 43 | EPHA1 | 13.21 | 0.01 |
| 44 | ERK7/MAPK15 | 13.46 | 0.06 |
| 45 | EPHB1 | 13.59 | 0 |
| 46 | ACK1 | 13.67 | 1.17 |
| 47 | CLK1 | 13.73 | 0.18 |
| 48 | CDK14/cyclin Y | 13.99 | 0.26 |
| | (PFTK1) | | |
| 49 | c-Kit | 14.17 | 0.13 |
| 50 | PHKg1 | 16.51 | 0.48 |
| 51 | TRKB | 16.53 | 2.93 |
| 52 | CDK16/cyclin Y | 17.54 | 0.47 |
| | (PCTAIRE) | | |
| 53 | LRRK2 | 17.75 | 0.47 |
| 54 | ASK1/MAP3K5 | 18.38 | 0.04 |
| 55 | CAMKK1 | 18.72 | 0.2 |
| 56 | YES/YES1 | 20.57 | 0.44 |
| 57 | ARK5/NUAK1 | 20.76 | 0.24 |
| 58 | MELK | 21.15 | 0.41 |
| 59 | CDK4/cyclin D2 | 21.84 | 0.01 |
| 60 | DYRK2 | 22.31 | 0.36 |
| 61 | TYRO3/SKY | 22.43 | 0.79 |
| 62 | CK1g1 | 22.87 | 0.06 |
| 63 | TAOK3/JIK | 23.06 | 0.76 |
| 64 | STK25/YSK1 | 23.14 | 2.06 |
| 65 | EPHB4 | 23.18 | 0.06 |
| 66 | TAOK2/TAO1 | 23.81 | 0.38 |
| 67 | CDK6/cyclin D1 | 24.24 | 1.18 |
| 68 | CK1g3 | 24.41 | 0.2 |
| 69 | PAK1 | 24.53 | 0.15 |
| 70 | NLK | 24.81 | 0.26 |
| 71 | AURORA C | 25.51 | 0.25 |
| 72 | CK1a1 | 26.46 | 0.04 |
| 73 | CK1g2 | 26.98 | 0.09 |
| 74 | ROS/ROS1 | 27.78 | 0.18 |
| 75 | MLK1/MAP3K9 | 27.88 | 0.14 |
| 76 | TAOK1 | 28.1 | 0.48 |
| 77 | STK38/NDR1 | 28.29 | 0.2 |
| 78 | CDK4/cyclin D3 | 28.6 | 0.09 |
| 79 | TRKA | 28.68 | 0.54 |
| 80 | CLK4 | 28.73 | 1.87 |
| 81 | FGFR1 | 28.82 | 0.14 |
| 82 | PAK3 | 29.09 | 0.45 |
| 83 | STK39/STLK3 | 29.59 | 0.54 |
| 84 | CK1epsilon | 29.82 | 0.1 |
| 85 | HPK1/MAP4K1 | 30.19 | 1.42 |
| 86 | SIK3 | 30.2 | 1.89 |
| 87 | FGFR3 | 30.57 | 0.89 |
| 88 | IRR/INSRR | 31.09 | 0.08 |
| 89 | EPHA7 | 31.31 | 0.39 |
| 90 | Aurora A | 33.41 | 0.44 |
| 91 | CDK4/cyclin D1 | 33.45 | 0.48 |
| 92 | STK33 | 33.46 | 0.48 |
| 93 | FGFR2 | 33.68 | 0.37 |
| 94 | CAMK1a | 33.96 | 0.83 |
| 95 | PDGFRb | 34.73 | 0.95 |
| 96 | TIE2/TEK | 34.75 | 0.15 |
| 97 | TXK | 34.85 | 0.7 |
| 98 | FMS | 35.44 | 0.6 |
| 99 | SIK2 | 35.86 | 0.55 |
| 100 | STK16 | 35.87 | 0.73 |
| 101 | FLT1/VEGFR1 | 36.29 | 0.65 |
| 102 | STK38L/NDR2 | 36.93 | 0.54 |
| 103 | STK22D/TSSK1 | 37.96 | 0.71 |
| 104 | MAST3 | 39.28 | 0.43 |
| 105 | LCK2/ICK | 39.59 | 1.56 |
| 106 | PKCb2 | 39.63 | 0.59 |
| 107 | FLT3 | 40.18 | 1.23 |
| 108 | LOK/STK10 | 41.43 | 0.02 |
| 109 | JNK1 | 41.61 | 0.45 |
| 110 | EPHA2 | 42.33 | 0.85 |
| 111 | FRK/PTK5 | 43.54 | 1.43 |
| 112 | HIPK4 | 43.93 | 0.88 |
| 113 | AXL | 44.17 | 0.59 |
| 114 | MST4 | 45.79 | 1.05 |
| 115 | HCK | 46.52 | 0.86 |
| 116 | BRK | 46.65 | 0.11 |
| 117 | PAK2 | 46.7 | 0.62 |
| 118 | MST3/STK24 | 47.02 | 0.03 |
| 119 | PYK2 | 47.5 | 0.49 |
| 120 | SYK | 48.39 | 0.8 |
| 121 | JNK3 | 48.68 | 0.23 |
| 122 | PKCg | 49.55 | 0.65 |
| 123 | GLK/MAP4K3 | 49.59 | 0.37 |
| 124 | GRK7 | 49.66 | 0.52 |
| 125 | DDR1 | 50.24 | 0.13 |
| 126 | ERN1/IRE1 | 50.78 | 0.16 |
| 127 | OSR1/OXSR1 | 50.8 | 1.75 |
| 128 | MLK3/MAP3K11 | 51.4 | 4.88 |
| 129 | CDK6/cyclin D2 | 51.47 | 2.11 |
| 130 | FER | 52.26 | 0.41 |
| 131 | BMPR2 | 52.43 | 1.68 |
| 132 | FAK/PTK2 | 52.49 | 3.13 |
| 133 | PDGFRa | 52.77 | 1.97 |
| 134 | CK1a1L | 53.1 | 2.14 |
| 135 | MST2/STK3 | 53.55 | 0.67 |
| 136 | RSK4 | 54.23 | 2.09 |
| 137 | CAMK1g | 54.26 | 1.19 |
| 138 | PKCnu/PRKD3 | 54.3 | 0.49 |
| 139 | SRPK1 | 54.32 | 0.28 |
| 140 | LYN B | 54.43 | 0.08 |
| 141 | FGR | 54.63 | 0.9 |
| 142 | CSK | 54.79 | 0.95 |
| 143 | TRKC | 54.91 | 1.29 |
| 144 | TBK1 | 55.03 | 0.06 |
| 145 | DCAMKL2 | 55.04 | 1.03 |
| 146 | ABL2/ARG | 56.13 | 0.46 |
| 147 | SIK1 | 56.31 | 2.6 |
| 148 | PHKg2 | 56.93 | 0.35 |
| 149 | PAK6 | 57.18 | 0.24 |
| 150 | STK32B/YANK2 | 57.59 | 3.62 |
| 151 | CAMK1d | 57.97 | 0.06 |
| 152 | IGF1R | 57.99 | 0.24 |
| 153 | RSK3 | 58.05 | 3.3 |
| 154 | GCK/MAP4K2 | 58.42 | 1.22 |
| 155 | DYRK3 | 58.57 | 0.1 |
| 156 | SNARK/NUAK2 | 59.04 | 1.36 |
| 157 | RSK1 | 59.15 | 1.78 |
| 158 | ALK | 59.66 | 2.12 |
| 159 | LIMK1 | 59.86 | 1.93 |
| 160 | MLCK2/MYLK2 | 59.95 | 3.03 |
| 161 | c-Src | 60.14 | 4.25 |
| 162 | SRPK2 | 60.7 | 0.18 |
| 163 | BRAF | 60.81 | 1 |
| 164 | ABL1 | 61.17 | 0.22 |
| 165 | CAMK2d | 61.72 | 3.7 |
| 166 | JNK2 | 61.73 | 0.16 |
| 167 | MST1/STK4 | 62.22 | 0.56 |
| 168 | MLK2/MAP3K10 | 62.32 | 0.29 |
| 169 | PKCa | 62.39 | 0.09 |
| 170 | MLCK/MYLK | 63.37 | 0.56 |
| 171 | CAMK2a | 64 | 1.12 |
| 172 | P38b/MAPK11 | 64.73 | 3.12 |
| 173 | IR | 65.1 | 0.64 |
| 174 | CAMK2b | 65.19 | 0.64 |
| 175 | ERN2/IRE2 | 65.36 | 0.25 |
| 176 | LYN | 65.69 | 1.2 |
| 177 | EPHB3 | 65.95 | 1.14 |
| 178 | RIPK2 | 66.14 | 0.25 |
| 179 | CAMK1b | 66.81 | 0.32 |
| 180 | BLK | 66.84 | 0.71 |
| 181 | MARK4 | 66.94 | 0.89 |
| 182 | TLK1 | 67.76 | 0.83 |
| 183 | RIPK5 | 67.77 | 1.36 |
| 184 | MYO3b | 68.11 | 3.3 |
| 185 | PLK1 | 68.29 | 1.42 |
| 186 | RAF1 | 68.57 | 0.42 |
| 187 | TNIK | 68.96 | 0 |
| 188 | RSK2 | 69.39 | 2.4 |
| 189 | SLK/STK2 | 69.49 | 0.35 |
| 190 | ULK1 | 70.32 | 0.56 |
| 191 | PKCmu/PRKD1 | 70.33 | 0.9 |
| 192 | c-MER | 70.99 | 1.1 |
| 193 | MYO3A | 71.31 | 3.16 |
| 194 | TEC | 71.5 | 2.01 |
| 195 | IKKa/CHUK | 71.52 | 1.75 |
| 196 | TLK2 | 72.17 | 1.09 |
| 197 | PKD2/PRKD2 | 72.27 | 0.53 |
| 198 | DCAMKL1 | 72.58 | 0.4 |
| 199 | ERBB4/HER4 | 73 | 1.58 |
| 200 | Aurora B | 73.04 | 0.3 |
| 201 | MEK3 | 73.46 | 2.86 |
| 202 | TYK1/LTK | 73.59 | 0.74 |
| 203 | ERBB2/HER2 | 74.2 | 0.45 |
| 204 | MARK2/PAR-1Ba | 74.34 | 1.44 |
| 205 | WNK3 | 74.47 | 0.62 |
| 206 | NIM1 | 75.97 | 1.33 |
| 207 | GRK1 | 76.35 | 1.78 |
| 208 | LIMK2 | 76.55 | 0.19 |
| 209 | LATS2 | 77.36 | 1.16 |
| 210 | IRAK1 | 79.02 | 2.57 |
| 211 | PRKX | 79.24 | 0.07 |
| 212 | DRAK1/STK17A | 79.29 | 0.26 |
| 213 | Haspin | 79.43 | 0.3 |
| 214 | RET | 79.45 | 2.04 |
| 215 | ARAF | 79.47 | 0.32 |
| 216 | CHK1 | 79.48 | 0.14 |
| 217 | PKCtheta | 79.79 | 0.97 |
| 218 | IKKe/IKBKE | 80.12 | 0.21 |
| 219 | MSK1/RPS6KA5 | 80.51 | 3.13 |
| 220 | GRK6 | 80.61 | 0.01 |
| 221 | ZIPK/DAPK3 | 80.76 | 1.08 |
| 222 | FYN | 81 | 1.7 |
| 223 | HIPK2 | 81.31 | 0.71 |
| 224 | MARK1 | 81.55 | 1.8 |
| 225 | PKN3/PRK3 | 81.55 | 2.69 |
| 226 | ITK | 81.68 | 3.73 |
| 227 | WNK1 | 82.51 | 0.47 |
| 228 | MSSK1/STK23 | 82.85 | 1.01 |
| 229 | MARK3 | 83.14 | 2.23 |
| 230 | PLK4/SAK | 83.4 | 0.55 |
| 231 | LCK | 83.64 | 0.26 |
| 232 | IKKb/IKBKB | 83.72 | 0.83 |
| 233 | EGFR | 83.73 | 2.46 |
| 234 | TESK2 | 83.74 | 2.44 |
| 235 | NEK5 | 83.83 | 0.92 |
| 236 | MEK5 | 84.06 | 2.53 |
| 237 | P38a/MAPK14 | 84.16 | 3.48 |
| 238 | CK2a | 84.21 | 2.43 |
| 239 | SGK2 | 84.68 | 0.4 |
| 240 | SGK1 | 84.89 | 2.3 |
| 241 | LATS1 | 85.02 | 0.51 |
| 242 | TSSK2 | 85.3 | 0.53 |
| 243 | PKN1/PRK1 | 85.42 | 1.13 |
| 244 | DAPK2 | 85.71 | 1.28 |
| 245 | PKN2/PRK2 | 85.76 | 1.59 |
| 246 | PKCIOTA | 86.06 | 1.91 |
| 247 | NEK3 | 86.56 | 1.12 |
| 248 | STK32C/YANK3 | 86.65 | 1.06 |
| 249 | PIM3 | 86.74 | 0.2 |
| 250 | RON/MST1R | 86.84 | 0.97 |
| 251 | SSTK/TSSK6 | 86.86 | 0.53 |
| 252 | MEKK1 | 87 | 2.81 |
| 253 | JAK2 | 87.03 | 0.6 |
| 254 | DDR2 | 87.23 | 0.74 |
| 255 | VRK1 | 87.31 | 2.28 |
| 256 | ULK2 | 87.32 | 2.78 |
| 257 | FGFR4 | 87.42 | 2.07 |
| 258 | CTK/MATK | 87.61 | 1.25 |
| 259 | PKCeta | 87.92 | 1.38 |
| 260 | GRK4 | 88.31 | 0.98 |
| 261 | KHS/MAP4K5 | 88.32 | 5.85 |
| 262 | PKAcg | 88.37 | 0.45 |
| 263 | KSR2 | 88.41 | 10.33 |
| 264 | KDR/VEGFR2 | 88.58 | 0.87 |
| 265 | MNK1 | 89.25 | 2.6 |
| 266 | DYRK4 | 89.29 | 0.4 |
| 267 | PKCb1 | 89.88 | 1.56 |
| 268 | MEKK2 | 90.02 | 0.34 |
| 269 | NEK9 | 90.05 | 2.62 |
| 270 | PKCzeta | 90.17 | 1.09 |
| 271 | WEE1 | 90.23 | 1.37 |
| 272 | DMPK | 90.4 | 0.13 |
| 273 | TYK2 | 90.71 | 0.7 |
| 274 | NEK2 | 91.1 | 1.01 |
| 275 | MNK2 | 91.2 | 3.12 |
| 276 | TNK1 | 91.26 | 0.83 |
| 277 | CK2a2 | 91.32 | 1.45 |
| 278 | GRK5 | 91.45 | 0.56 |
| 279 | TSSK3/STK22C | 91.47 | 1.2 |
| 280 | ULK3 | 91.77 | 2.71 |
| 281 | JAK1 | 92.07 | 0.82 |
| 282 | NEK6 | 92.11 | 0.84 |
| 283 | p70S6K/RPS6KB1 | 92.14 | 0.16 |
| 284 | p70S6Kb/RPS6KB2 | 92.21 | 3.07 |
| 285 | ROCK2 | 92.25 | 0.53 |
| 286 | NEK1 | 92.29 | 0.19 |
| 287 | DMPK2 | 92.41 | 1.88 |
| 288 | SGK3/SGKL | 92.63 | 2.05 |
| 289 | BRSK1 | 92.76 | 0.22 |
| 290 | SNRK | 92.96 | 2.34 |
| 291 | HIPK1 | 93 | 4.51 |
| 292 | PKA | 93.14 | 1.67 |
| 293 | TESK1 | 93.14 | 2.74 |
| 294 | NEK7 | 93.22 | 2.1 |
| 295 | PBK/TOPK | 93.48 | 1.45 |
| 296 | PIM2 | 93.84 | 1.97 |
| 297 | EPHA8 | 93.99 | 0.13 |
| 298 | MEKK3 | 94.01 | 1.82 |
| 299 | PKCepsilon | 94.07 | 1.5 |
| 300 | HIPK3 | 94.08 | 1.55 |
| 301 | NEK11 | 94.25 | 0.17 |
| 302 | PLK3 | 94.42 | 0.43 |
| 303 | SBK1 | 94.45 | 1.4 |
| 304 | TGFBR2 | 94.46 | 0.69 |
| 305 | CLK3 | 94.59 | 2.7 |
| 306 | IRAK4 | 94.79 | 7.08 |
| 307 | AKT1 | 94.93 | 0.55 |
| 308 | PKCd | 95.16 | 1.28 |
| 309 | CAMK4 | 95.25 | 2.97 |
| 310 | MKK6 | 95.42 | 0.25 |
| 311 | NEK4 | 95.76 | 2.35 |
| 312 | AKT3 | 96.1 | 0.79 |
| 313 | PASK | 96.32 | 0.51 |
| 314 | MKK7 | 96.35 | 0.92 |
| 315 | c-MET | 96.37 | 0.7 |
| 316 | MKK4 | 96.38 | 1.78 |
| 317 | VRK2 | 96.56 | 0.91 |
| 318 | MRCKa/CDC42BPA | 96.62 | 0.35 |
| 319 | YSK4/MAP3K19 | 96.76 | 0.65 |
| 320 | SRMS | 96.77 | 1.07 |
| 321 | ROCK1 | 97.22 | 1.07 |
| 322 | TTBK2 | 97.3 | 1.37 |
| 323 | BRSK2 | 97.46 | 0.17 |
| 324 | IRAK2 | 97.48 | 1.57 |
| 325 | ZAK/MLTK | 97.5 | 0.03 |
| 326 | PKG1b | 97.63 | 3.74 |
| 327 | ERK5/MAPK7 | 97.7 | 2.45 |
| 328 | RIPK4 | 97.79 | 0.12 |
| 329 | PIM1 | 97.81 | 0.89 |
| 330 | STK21/CIT | 97.88 | 1.96 |
| 331 | TAK1 | 98.18 | 0.9 |
| 332 | CDC7/DBF4 | 98.19 | 0.99 |
| 333 | GRK2 | 99.08 | 0.45 |
| 334 | MRCKb/CDC42BPB | 99.29 | 2.71 |
| 335 | PKG2/PRKG2 | 99.49 | 4.09 |
| 336 | DAPK1 | 99.7 | 11.89 |
| 337 | JAK3 | 99.71 | 0.64 |
| 338 | AKT2 | 100.11 | 0.29 |
| 339 | ZAP70 | 100.12 | 1.87 |
| 340 | KSR1 | 100.29 | 0.86 |
| 341 | MAPKAPK3 | 100.55 | 0.94 |
| 342 | MINK/MINK1 | 101.34 | 1.36 |
| 343 | GRK3 | 101.52 | 0.91 |
| 344 | COT1/MAP3K8 | 102.29 | 1.04 |
| 345 | MAPKAPK2 | 102.39 | 2.26 |
| 346 | P38d/MAPK13 | 102.41 | 3.57 |
| 347 | BTK | 102.6 | 2.77 |
| 348 | HGK/MAP4K4 | 102.7 | 0.21 |
| 349 | MASTL | 102.89 | 0.2 |
| 350 | MAPKAPK5/PRAK | 103.45 | 0.37 |
| 351 | PKAcb | 104.22 | 0.73 |
| 352 | CAMK2g | 105.12 | 0.39 |
| 353 | PDK1/PDPK1 | 105.14 | 0.74 |
| 354 | ALK1/ACVRL1 | 105.15 | 2.8 |
| 355 | MYLK3 | 105.4 | 1.73 |
| 356 | PLK2 | 106.76 | 3.2 |
| 357 | PKG1a | 107.68 | 3.02 |
| 358 | TTBK1 | 107.7 | 2.54 |
| 359 | WNK2 | 107.75 | 1.34 |
| 360 | MEKK6 | 109.01 | 1.6 |
| 361 | ALK5/TGFBR1 | 111.87 | 0.47 |
| 362 | MLK4 | 114.37 | 0.69 |
| 363 | MSK2/RPS6KA4 | 115.01 | 1.99 |
| 364 | ALK6/BMPR1B | 115.28 | 10.57 |
| 365 | ALK2/ACVR1 | 117.89 | 6.15 |
| 366 | P38g | 118.19 | 2.58 |
| 367 | ALK3/BMPR1A | 125.62 | 1.56 |
| 368 | ALK4/ACVR1B | 132.41 | 0.08 |
| 369 | BMX/ETK | 141.45 | 1.24 |
| 370 | MEK1 | 149.45 | 0.19 |
| 371 | MEK2 | 175.95 | 2.97 |

### Experimental Example 6. Evaluation of In Vitro Liver Metabolic Stability and Inhibitory Activity Against Five Representative CYPs

5 derivatives were evaluated for in vitro metabolic stability in liver microsomes from three different species (human, dog, mouse) and for inhibitory activity against 5 representative cytochrome P450 enzymes (CYPs)(Table 3). In terms of liver microsomal stability, the 2′-pyridyl group is more suitable than the α-methyl-4′-pyridyl group as the terminal aromatic group at position 6, and the aminopyrimidyl group is better than the aminopyridyl group as a substituent at position 2. Except for CYP3A4, the five derivatives showed similar inhibitory activity against the remaining major CYPs, indicating that they could be applied as co-administered agents with other agents by showing desirable CYP inhibition profiles. Among them, the derivatives including an aminopyrimidine group at position 2 showed only slight inhibition against all five CYPs, indicating that aminopyrimidine is the most suitable substitution at position 2 to avoid inhibition of CYPs. Among the derivatives, Compound 54 was shown to be the best derivative in terms of liver metabolic stability and preservation of CYP activity.

TABLE 3
In vitro liver microsomal metabolic stability (% remaining
amount) and five CYP inhibitory activity (% activity)

| | Liver microsomal | | | | | | | |
|---|---|---|---|---|---|---|---|---|
| | metabolic stability | | | CYP % activity at 10 μM | | | | |
| | (% remaining amount) | | | concentration | | | | |
| Example | Human | Dog | Mouse | 1A2 | 2C9 | 2C19 | 2D6 | 3A4 |
| 49 | 32.2 | 45.1 | 35.3 | 59.3 | 73.0 | 76.8 | 89.6 | 38.9 |
| 50 | 40.6 | 53.6 | 45.0 | 74.9 | 82.9 | 75.4 | 85.7 | 72.4 |
| 51 | 27.3 | 79.5 | 34.0 | 68.6 | 67.5 | 73.9 | 88.8 | 31.8 |
| 53 | 64.8 | 46.2 | 49.9 | 74.7 | 57.9 | 68.1 | 81.0 | 43.0 |
| 54 | 100 | 67.6 | 85.0 | 87.6 | 81.6 | 72.6 | 92.1 | 92.8 |

### Experimental Example 7. Efficacy in In Vivo Breast Cancer Mouse Model

A mouse efficacy experiment was conducted after obtaining approval from the Laboratory Animal Ethics Committee of GenNBio (Review No.: GN-IACUC-Research 22-02-07). SK-Br3 and HCC-1954 cells (1×10⁶ cells each) mixed with Matrigel (BD Biosciences) were stereotactically injected into 5-week-old female Balb/c nude mice (Orient Bio, Seongnam, Republic of Korea). After tumors reached a size of about 100 mm³, the compound, trastuzumab (20 mg/kg), and the like were intraperitoneally injected into mice (n=8 per group)(vehicle: 8% DMSO, 4% Tween-80, 88% PBS). The tumor size was measured twice weekly for about 4 weeks and calculated as follows. Volume (mm³)=(a×b²)/2; “a”, maximum diameter; “b”, vertical diameter

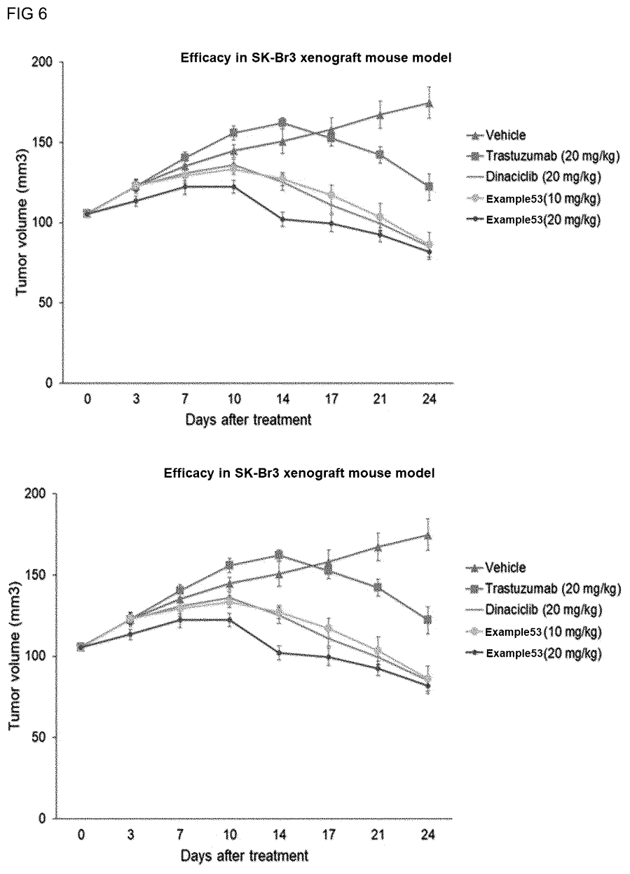

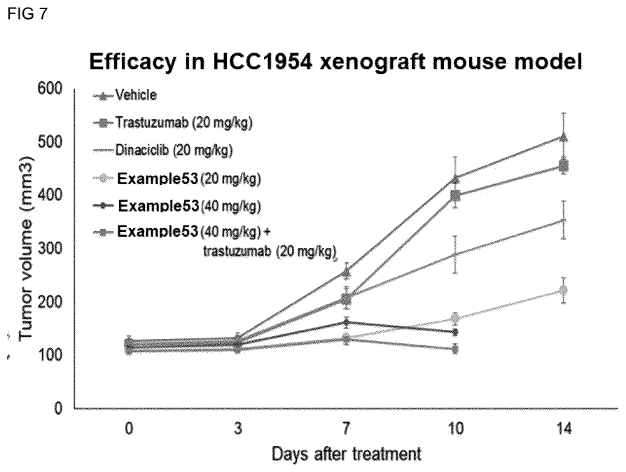

The excellent anticancer efficacy of Example 53 against SK-Br3 and HCC1954 was observed in the xenograft models. In the SK-Br3 xenograft model, Example 53 showed anticancer efficacy in a dose-dependent manner without any change in mouse weight upon administration at 10 mg/kg and 20 mg/kg, with both showing a level of anticancer efficacy similar to that of dinaciclib administered at 20 mg/kg. Above all, the administration of dinaciclib at 20 mg/kg showed toxicity with a decrease in body weight in mice, but Example 53 showed an excellent result that even upon administration at 10 mg/kg and 20 mg/kg, the weight of the mice was maintained similarly compared to the vehicle-treated group (FIG. 6). In addition, in the HCC1954 xenograft model, Example 53 showed dose-dependent anticancer efficacy upon administration at 20 mg/kg and 40 mg/kg, both of which showed better efficacy than dinaciclib at 20 mg/kg (FIG. 7).

Although the present invention has been described in detail above through preferred preparation examples, example compounds and experimental examples, the scope of the present invention is not limited to the specific example compounds, and should be interpreted by the appended claims. Furthermore, it is to be understood that a person with ordinary skill in the art can make many modifications and variations without departing from the scope of the present invention.

1. A compound of the following Chemical Formula 1, a stereoisomer thereof, a hydrate thereof, a solvate thereof, or a pharmaceutically acceptable salt thereof: 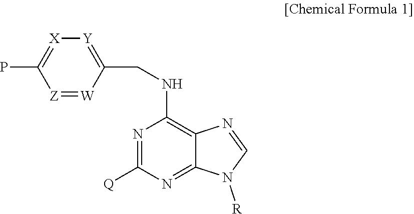
X, Y, Z, and W are each independently CH or N;
P is phenyl or a 5- to 12-membered heteroaryl comprising one or more heteroatoms of N, S and O, and wherein the phenyl or heteroaryl is unsubstituted or substituted with one or more C₁₋₆ straight or branched alkyls;
Q is phenyl, a 5- to 12-membered heterocycloalkyl comprising one or more heteroatoms of N, S and O, or a 5- to 12-membered heteroaryl comprising one or more heteroatoms of N, S and O, wherein the phenyl, the heterocycloalkyl or the heteroaryl is optionally substituted with one or more non-hydrogen substituents of a C₁₋₆ straight or branched alkyl, a C₁₋₆ alkoxy, a hydroxyl, a halogen, oxo (═O), —NR¹R², and —CONH₂, wherein the C₁₋₆ straight or branched alkyl is optionally substituted with a hydroxyl, and wherein the R¹ and R² are each independently hydrogen, or a C₁₋₆ straight or branched alkyl unsubstituted or substituted with a hydroxyl; and
R is a C₁₋₆ straight or branched alkyl, a C₃₋₆ cycloalkyl, or a 3- to 12-membered heterocycloalkyl comprising one or more heteroatoms of N, S and O.

2. The compound, the stereoisomer thereof, the hydrate thereof, the solvate thereof, or the pharmaceutically acceptable salt thereof of claim 1,
wherein one of X, Y, Z and W is N, and the others are CH,
P is phenyl or a 5- to 12-membered heteroaryl comprising at least one N as a heteroatom, and wherein the phenyl or heteroaryl is unsubstituted or substituted with one or more C₁₋₃ straight or branched alkyls,
Q is phenyl, a 5- to 12-membered heterocycloalkyl comprising one or more heteroatoms of N and O, or a 5- to 12-membered heteroaryl comprising at least one N as a heteroatom, wherein the phenyl, the heterocycloalkyl or the heteroaryl is optionally substituted with one or more non-hydrogen substituents of a C₁₋₃ straight or branched alkyl, a C₁₋₃ alkoxy, a hydroxyl, a halogen, oxo (═O), —NR¹R², and —CONH₂, wherein the C₁₋₃ straight or branched alkyl is optionally substituted with a hydroxyl, and wherein the R¹ and R² are each independently hydrogen or a C₁₋₃ straight or branched alkyl unsubstituted or substituted with a hydroxyl; and
R is a C₁₋₃ straight or branched alkyl, a C₃₋₆ cycloalkyl, or a 3- to 6-membered heterocycloalkyl comprising at least one O as a heteroatom.

3. The compound, the stereoisomer thereof, the hydrate thereof, the solvate thereof, or the pharmaceutically acceptable salt thereof of claim 1,
wherein one of X, Y, Z and W is N, and the others are CH,
P is phenyl, pyridine, pyrazole, furan, thiophene, thiazole or indazole, and wherein the phenyl, pyridine, pyrazole, furan, thiophene, thiazole or indazole is unsubstituted or substituted with one or more C₁₋₃ straight or branched alkyls,
Q is phenyl, pyridine, pyrazole, triazole, indole, indazole, benzimidazole, quinoline, thiazole, pyrimidine, morpholine or piperidine, wherein the phenyl, pyridine, pyrazole, triazole, indole, indazole, benzimidazole, quinoline, thiazole, pyrimidine, morpholine or piperidine is optionally substituted with a C₁₋₃ straight or branched alkyl, a C₁₋₃ alkoxy, a hydroxyl, a halogen, oxo (═O), —NR¹R², and —CONH₂, wherein the C₁₋₃ straight or branched alkyl is optionally substituted with a hydroxyl, and R¹ and R² are each independently hydrogen or a C₁₋₃ straight or branched alkyl substituted with a hydroxyl; and
R is a C₁₋₃ straight or branched alkyl, a C₃₋₆ cycloalkyl, or oxane.

4. The compound, the stereoisomer thereof, the hydrate thereof, the solvate thereof, or the pharmaceutically acceptable salt thereof of claim 3,
wherein when Q is phenyl, the phenyl is unsubstituted or substituted with a hydroxyl, —NH₂, —NHR³, or —CONH₂, and wherein R³ is a C₁₋₃ hydroxyalkyl,
when Q is pyridine, pyrazole, triazole, indole, indazole, benzimidazole, quinoline, thiazole, or pyrimidine, wherein the pyridine, pyrazole, triazole, indole, indazole, benzimidazole, quinoline, thiazole, or pyrimidine is unsubstituted or substituted with one or more non-hydrogen substituents of a C₁₋₃ straight or branched alkyl, a C₁₋₃ alkoxy, a halogen, oxo (═O), —NH₂, —NHR⁴, and —CONH₂, wherein the C₁₋₃ straight or branched alkyl is optionally substituted with a hydroxyl, and R⁴ is a C₁₋₃ hydroxyalkyl, and
when Q is morpholine or piperidine, the morpholine or piperidine is unsubstituted or substituted with a C₁₋₃ hydroxyalkyl.

5. The compound, the stereoisomer thereof, the hydrate thereof, the solvate thereof, or the pharmaceutically acceptable salt thereof of claim 1, wherein the compound of Chemical Formula 1 is any one compound of the following (1) to (66):
(1) N-([2,3′-bipyridin]-6′-ylmethyl)-9-isopropyl-2-(pyridin-3-yl)-9H-purin-6-amine;
(2) N-([3,3′-bipyridin]-6-ylmethyl)-9-isopropyl-2-(pyridin-3-yl)-9H-purin-6-amine;
(3) 9-isopropyl-N-((6′-methyl-[3,3′-bipyridin]-6-yl)methyl)-2-(pyridin-3-yl)-9H-purin-6-amine;
(4) N-([3,4′-bipyridin]-6-ylmethyl)-9-isopropyl-2-(pyridin-3-yl)-9H-purin-6-amine;
(5) 9-isopropyl-N-((2′-methyl-[3,4′-bipyridin]-6-yl)methyl)-2-(pyridin-3-yl)-9H-purin-6-amine;
(6) N-([2,3′-bipyridin]-5-ylmethyl)-9-isopropyl-2-(pyridin-3-yl)-9H-purin-6-amine;
(7) N-([2,4′-bipyridin]-5-ylmethyl)-9-isopropyl-2-(pyridin-3-yl)-9H-purin-6-amine;
(8) N-([2,3′-bipyridin]-5-ylmethyl)-9-isopropyl-2-phenyl-9H-purin-6-amine;
(9) 3-(6-(([2,3′-bipyridin]-5-ylmethyl)amino)-9-isopropyl-9H-purin-2-yl)benzamide;
(10) 4-(6-(([2,3′-bipyridin]-5-ylmethyl)amino)-9-isopropyl-9H-purin-2-yl)benzamide;
(11) N-([2,3′-bipyridin]-5-ylmethyl)-2-(1H-indol-5-yl)-9-isopropyl-9H-purin-6-amine;
(12) N-([2,3′-bipyridin]-5-ylmethyl)-2-(1H-indol-6-yl)-9-isopropyl-9H-purin-6-amine;
(13) N-([2,3′-bipyridin]-5-ylmethyl)-2-(1H-indazol-6-yl)-9-isopropyl-9H-purin-6-amine;
(14) N-([2,3′-bipyridin]-5-ylmethyl)-2-(1H-indazol-5-yl)-9-isopropyl-9H-purin-6-amine;
(15) 5-(6-(([2,3′-bipyridin]-5-ylmethyl)amino)-9-isopropyl-9H-purin-2-yl)-1,3-dihydro-2H-benzo[d]imidazol-2-one;
(16) N-([2,3′-bipyridin]-5-ylmethyl)-9-isopropyl-2-(quinolin-3-yl)-9H-purin-6-amine;
(17) N-([2,3′-bipyridin]-5-ylmethyl)-9-isopropyl-2-(6-methylpyridin-3-yl)-9H-purin-6-amine;
(18) N-([2,3′-bipyridin]-5-ylmethyl)-2-(6-aminopyridin-3-yl)-9-isopropyl-9H-purin-6-amine;
(19) N-([2,3′-bipyridin]-5-ylmethyl)-2-(2-aminopyridin-4-yl)-9-isopropyl-9H-purin-6-amine;
(20) N-([2,3′-bipyridin]-5-ylmethyl)-9-isopropyl-2-(pyrimidin-5-yl)-9H-purin-6-amine;
(21) N-([2,3′-bipyridin]-5-ylmethyl)-2-(2-aminopyrimidin-5-yl)-9-isopropyl-9H-purin-6-amine;
(22) N-([2,3′-bipyridin]-5-ylmethyl)-9-isopropyl-2-(1H-pyrazol-5-yl)-9H-purin-6-amine;
(23) N-([2,3′-bipyridin]-5-ylmethyl)-9-isopropyl-2-(1H-pyrazol-4-yl)-9H-purin-6-amine;
(24) N-([2,3′-bipyridin]-5-ylmethyl)-9-isopropyl-2-(thiazol-5-yl)-9H-purin-6-amine;
(25) N-([2,3′-bipyridin]-5-ylmethyl)-2-(2-aminophenyl)-9-isopropyl-9H-purin-6-amine;
(26) N-([2,3′-bipyridin]-5-ylmethyl)-2-(3-aminophenyl)-9-isopropyl-9H-purin-6-amine;
(27) N-([2,3′-bipyridin]-5-ylmethyl)-2-(4-aminophenyl)-9-isopropyl-9H-purin-6-amine;
(28) N-([2,3′-bipyridin]-5-ylmethyl)-2-(6-fluoropyridin-3-yl)-9-isopropyl-9H-purin-6-amine;
(29) N-([2,3′-bipyridin]-5-ylmethyl)-2-(2-fluoropyridin-4-yl)-9-isopropyl-9H-purin-6-amine;
(30) N-([2,3′-bipyridin]-5-ylmethyl)-2-(2-fluoropyridin-3-yl)-9-isopropyl-9H-purin-6-amine;
(31) 2-((2-(6-(([2,3′-bipyridin]-5-ylmethyl)amino)-9-isopropyl-9H-purin-2-yl)phenyl)amino)ethan-1-ol,
(32) 3-((2-(6-(([2,3′-bipyridin]-5-ylmethyl)amino)-9-isopropyl-9H-purin-2-yl)phenyl)amino)propan-1-ol;
(33) 3-((3-(6-(([2,3′-bipyridin]-5-ylmethyl)amino)-9-isopropyl-9H-purin-2-yl)phenyl)amino)propan-1-ol;
(34) 3-((4-(6-(([2,3′-bipyridin]-5-ylmethyl)amino)-9-isopropyl-9H-purin-2-yl)phenyl)amino)propan-1-ol;
(35) 2-((5-(6-(([2,3′-bipyridin]-5-ylmethyl)amino)-9-isopropyl-9H-purin-2-yl)pyridin-2-yl)amino)ethan-1-ol;
(36) 3-((5-(6-(([2,3′-bipyridin]-5-ylmethyl)amino)-9-isopropyl-9H-purin-2-yl)pyridin-2-yl)amino)propan-1-ol,
(37) 2-((4-(6-(([2,3′-bipyridin]-5-ylmethyl)amino)-9-isopropyl-9H-purin-2-yl)pyridin-2-yl)amino)ethan-1-ol;
(38) 3-((4-(6-(([2,3′-bipyridin]-5-ylmethyl)amino)-9-isopropyl-9H-purin-2-yl)pyridin-2-yl)amino)propan-1-ol;
(39) 3-((3-(6-(([2,3′-bipyridin]-5-ylmethyl)amino)-9-isopropyl-9H-purin-2-yl)pyridin-2-yl)amino)propan-1-ol;
(40) N-([2,3′-bipyridin]-5-ylmethyl)-2-(5-amino-3-methyl-1H-pyrazol-1-yl)-9-isopropyl-9H-purin-6-amine;
(41) (1-(6-(([2,3′-bipyridin]-5-ylmethyl)amino)-9-isopropyl-9H-purin-2-yl)-1H-1,2,3-triazol-4-yl)methanol;
(42) 2-(1-(6-(([2,3′-bipyridin]-5-ylmethyl)amino)-9-isopropyl-9H-purin-2-yl)-1H-1,2,3-triazol-4-yl)ethan-1-ol;
(43) 3-(6-(([2,3′-bipyridin]-6′-ylmethyl)amino)-9-isopropyl-9H-purin-2-yl)phenol;
(44) N-([2,3′-bipyridin]-6′-ylmethyl)-9-isopropyl-2-(5-methoxypyridin-3-yl)-9H-purin-6-amine;
(45) N-([2,3′-bipyridin]-6′-ylmethyl)-2-(6-aminopyridin-3-yl)-9-isopropyl-9H-purin-6-amine;
(46) N-([2,3′-bipyridin]-5-ylmethyl)-9-ethyl-2-(pyridin-3-yl)-9H-purin-6-amine;
(47) N-([2,3′-bipyridin]-5-ylmethyl)-9-cyclopentyl-2-(pyridin-3-yl)-9H-purin-6-amine;
(48) N-([2,3′-bipyridin]-5-ylmethyl)-2-(pyridin-3-yl)-9-(tetrahydro-2H-pyran-4-yl)-9H-purin-6-amine;
(49) 2-(6-aminopyridin-3-yl)-9-ethyl-N-((2′-methyl-[2,4′-bipyridin]-5-yl)methyl)-9H-purin-6-amine;
(50) 2-(2-aminopyrimidin-5-yl)-9-ethyl-N-((2′-methyl-[2,4′-bipyridin]-5-yl)methyl)-9H-purin-6-amine;
(51) 9-ethyl-N-((2′-methyl-[2,4′-bipyridin]-5-yl)methyl)-2-(pyrimidin-5-yl)-9H-purin-6-amine;
(52) 5-(9-ethyl-6-(((2′-methyl-[2,4′-bipyridin]-5-yl)methyl)amino)-9H-purin-2-yl)-1,3-dihydro-2H-benzo[d]imidazol-2-one;
(53) N-([2,2′-bipyridin]-5-ylmethyl)-2-(6-aminopyridin-3-yl)-9-ethyl-9H-purin-6-amine;
(54) N-([2,2′-bipyridin]-5-ylmethyl)-2-(2-aminopyrimidin-5-yl)-9-ethyl-9H-purin-6-amine;
(55) 9-ethyl-2-(pyridin-3-yl)-N-((6-(thiazol-2-yl)pyridin-3-yl)methyl)-9H-purin-6-amine;
(56) N-((6-(1H-pyrazol-4-yl)pyridin-3-yl)methyl)-9-ethyl-2-(pyridin-3-yl)-9H-purin-6-amine;
(57) N-((6-(1H-indazol-6-yl)pyridin-3-yl)methyl)-9-ethyl-2-(pyridin-3-yl)-9H-purin-6-amine;
(58) 9-ethyl-N-((6-(furan-3-yl)pyridin-3-yl)methyl)-2-(pyridin-3-yl)-9H-purin-6-amine;
(59) N-((6-(1H-pyrazol-1-yl)pyridin-3-yl)methyl)-9-ethyl-2-(pyridin-3-yl)-9H-purin-6-amine;
(60) N-((6-(3,5-dimethyl-1H-pyrazol-1-yl)pyridin-3-yl)methyl)-9-ethyl-2-(pyridin-3-yl)-9H-purin-6-amine;
(61) 2-(6-aminopyridin-3-yl)-N-((6-(3,5-dimethyl-1H-pyrazol-1-yl)pyridin-3-yl)methyl)-9-ethyl-9H-purin-6-amine;
(62) 2-(2-aminopyrimidin-5-yl)-N-((6-(3,5-dimethyl-1H-pyrazol-1-yl)pyridin-3-yl)methyl)-9-ethyl-9H-purin-6-amine;
(63) (R)-2-(4-(6-(([2,3′-bipyridin]-5-ylmethyl)amino)-9-isopropyl-9H-purin-2-yl)morpholin-3-yl)ethan-1-ol;
(64) (S)-2-(1-(9-isopropyl-6-(((2′-methyl-[2,4′-bipyridin]-5-yl)methyl)amino)-9H-purin-2-yl)piperidin-2-yl)ethan-1-ol;
(64) (S)-2-(1-(9-isopropyl-6-(((2′-methyl-[2,4′-bipyridin]-5-yl)methyl)amino)-9H-purin-2-yl)piperidin-2-yl)ethan-1-ol;
(65) (S)-2-(1-(6-(((6-(3,5-dimethyl-1H-pyrazol-1-yl)pyridin-3-yl)methyl)amino)-9-ethyl-9H-purin-2-yl)piperidin-2-yl)ethan-1-ol; and
(66) (R)-2-(4-(6-(((6-(3,5-dimethyl-1H-pyrazol-1-yl)pyridin-3-yl)methyl)amino)-9-ethyl-9H-purin-2-yl)morpholin-3-yl)ethan-1-ol.

6. A method for preparing a compound of Chemical Formula 1, the method comprising: preparing a compound of Chemical Formula 3 from a compound of Chemical Formula 2;
preparing a compound of Chemical Formula 4 from the compound of Chemical Formula 3; and
preparing the compound of Chemical Formula 1 from the compound of Chemical Formula 4: 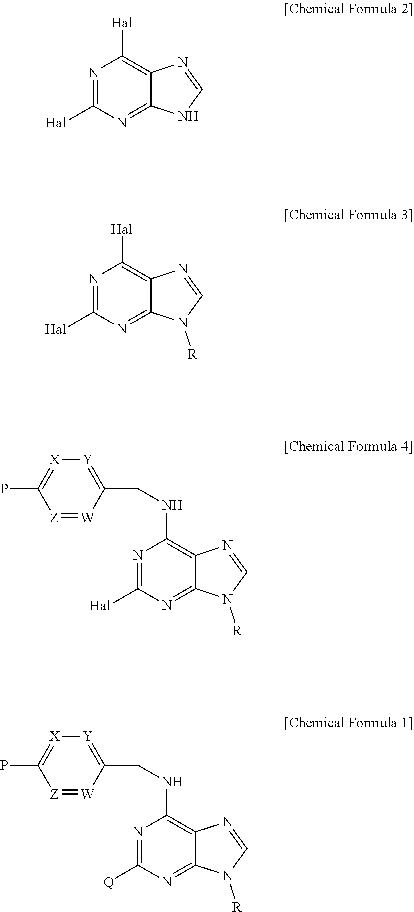
in the above formulae, Hal is a halogen which is a leaving group, and X, Y, Z, W, P, Q, and R are each the same as defined above.

7. A pharmaceutical composition for preventing or treating cancer, comprising the compound of Chemical Formula 1, the isomer thereof, the solvate thereof, the hydrate thereof, or the pharmaceutically acceptable salt thereof of claim 1 as an active ingredient; and a pharmaceutically acceptable carrier.

8. The pharmaceutical composition of claim 7, wherein the compound of Chemical Formula 1 inhibits human epidermal growth factor receptor 2 (HER2).

9. The pharmaceutical composition of claim 7, wherein the cancer is one or more selected from the group consisting of colorectal cancer, gastric cancer, lung cancer, biliary tract cancer, bladder cancer, esophageal cancer, melanoma, ovarian cancer, liver cancer, prostate cancer, pancreatic cancer, colon cancer, head and neck cancer, uterine cancer, breast cancer, and cervical cancer.

10. A method for preventing or treating cancer, the method comprising administering the compound of Chemical Formula 1, the stereoisomer thereof, the hydrate thereof, the solvate thereof, or the pharmaceutically acceptable salt thereof of claim 1 to a subject in need thereof.

11. A use of the compound of Chemical Formula 1, the stereoisomer thereof, the hydrate thereof, the solvate thereof, or the pharmaceutically acceptable salt thereof of claim 1 for preventing or treating cancer.

12. A use of the compound of Chemical Formula 1, the stereoisomer thereof, the hydrate thereof, the solvate thereof, or the pharmaceutically acceptable salt thereof of claim 1 for use in the preparation of a drug for preventing or treating cancer.
